(19) United States
(12) Patent Application Publication
(10) Pub. No.: US 2023/0407397 A1
(43) Pub. Date: Dec. 21, 2023

(54) METHODS FOR IDENTIFYING DRIVERS OF IMMUNE RESPONSES USING MOLECULAR EXPRESSION SIGNATURES OF IMMUNOMODULATING AGENTS

(71) Applicants: Massachusetts Institute of Technology, Cambridge, MA (US); The General Hospital Corporation, Boston, MA (US)

(72) Inventors: Nir Hacohen, Cambridge, MA (US); Ang Cui, Cambridge, MA (US)

(73) Assignees: Massachusetts Institute of Technology, Cambridge, MA (US); The General Hospital Corporation, Boston, MA (US)

(21) Appl. No.: 18/035,269

(22) PCT Filed: Nov. 4, 2021
(86) PCT No.: PCT/US21/58074

§ 371 Date: May 3, 2023

Related U.S. Application Data
(60) Provisional application No. 63/109,795, filed on Nov. 4, 2020.

(51) Int. Cl. C12Q 1/6881; C12Q 1/6809; A61K 45/06

(52) U.S. Cl. CPC C12Q 1/6881; C12Q 1/6809; A61K 45/06; C12Q 2600/136

(57) ABSTRACT

The present disclosure provides methods for determining signatures of immune responses to immunomodulating agents in cells and in tissues. Also provided herein are computational methods for deconvoluting gene expression profiling data. Further provided herein are methods for treating a disease or disorder (e.g, proliferative diseases such as cancer, autoimmune diseases such as rheumatoid arthritis and psoriasis, allergies, etc.) in a subject comprising administering one or more immunomodulating agents to drive an immune response, in combination with a treatment for the disease or disorder (e.g, an anti-cancer therapy, an anti-viral therapy, a vaccination, etc.).

### RELATED APPLICATIONS

This application claims priority under 35 U.S.C. § 119(e) to U.S. Provisional Application, U.S. Ser. No. 63/109,795, filed Nov. 4, 2020, which is incorporated herein by reference.

### GOVERNMENT SUPPORT

This invention was made with government support under grant number P50 HG006193 awarded by National Human Genome Research Institute of the National Institutes of Health. The government has certain rights in the invention.

### BACKGROUND OF THE INVENTION

Immunomodulating agents such as cytokines mediate complex cellular signaling networks and play a central role in health and disease. Dysregulation of immune responses is associated with a multitude of conditions. During an immune response, immunomodulating agents such as cytokines are secreted by cells and bind to cognate receptors locally or at a distance, triggering downstream signaling pathways and orchestrating coordinated actions among different cell types. While many individual cytokines and other immunomodulating agents have been studied in depth and are used directly as therapies or targeted with inhibitors, there is a need for a systems-level framework for reconstructing cell-cell communication networks mediated by cytokines and other immunomodulating agents to define immune responses as a coordinated set of cell activation events.

Systems-level approaches require simultaneous measurement of many parameters in a biological system and are therefore promising for enhancing our understanding of cytokine actions and cell-cell communication networks. With advances in single cell RNA sequencing (scRNA-seq) technologies, many cells in a microenvironment can now be profiled simultaneously to characterize cell types and their states and programs. However, while gene expression data reveal the functional states of thousands of cells, the data do not reveal what factors trigger the observed cell states and their functions. Previous studies have inferred cell-cell communications in transcriptomic studies using ligand and receptor expression but have limited value because cytokine receptor expression is not an accurate predictor of cytokine responses. Furthermore, such approaches do not reveal how immunomodulating agents such as cytokines control specific biological processes in each cell type.

There is a need to develop a global view of cell-type-specific responses to each immunomodulating agent to elucidate how they orchestrate cell-cell communication networks in a complex immune response. With these data, major biological processes regulated by such immunomodulating agents could be identified and manipulated for the treatment of immune-associated diseases.

### SUMMARY OF THE INVENTION

To address this need, methods were developed for profiling gene expression of cell populations in response to various immunomodulating agents (e.g., cytokines), allowing a compendium of cell-type-specific cytokine signatures to be elucidated. The methods involve injecting an immunomodulating agent into a population of immune cells either in vitro or in vivo and then harvesting the immune cells. Following profiling of gene expression in the cells, cell type and gene expression data can be correlated and compared across various immunomodulating agents. An “Immune Dictionary” was developed using the methods described herein. This dictionary can be used to predict, classify, diagnose, and/or treat any diseases or conditions involving the immune system. Computational algorithms have also been developed and used for comparing gene expression signatures across various cell types and immunomodulating agents, as well as data obtained from an immune response to identify drivers of the response and therapeutic targets. This compendium (or “dictionary”) was used to systematically compare cytokine and vaccine adjuvant responses across cell types and deconvolve complex immune responses. The methods disclosed herein provide a global view of cell-type specific cytokine responses. This framework has been used to study vaccination and cancer immunotherapy, and drivers that can be used to enhance vaccine or therapeutic responses have been found. The framework can be readily applied to other high-throughput molecular datasets for assessing the roles of cytokines and other immunomodulating agents and cell-cell communication networks in any immune response. The information obtained from the methods disclosed herein is also useful for the discovery of new therapeutic targets and approaches for immune-related diseases, as well as for driving immune responses concurrently with a disease treatment to enhance therapeutic efficacy. For example, the methods disclosed herein have shown that IL18 is useful for enhancing cancer therapy.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows a schematic of an example of the methods for determining signatures of immune responses to immunomodulating agents (in this example, cytokines). Cytokines are injected into distinct mice (n=3 per cytokine). Cells from the draining lymph nodes of the mice are harvested and processed, and single cell RNA-seq is performed. Cell-type-specific cytokine gene expression signatures are computed, and these are compared across all cytokines tested and cell types present within the sample to identify biological features of cell type-specific responses to individual cytokines. Computational methods can also be used to infer cell-cell communication.

FIG. 2 shows an example of the cytokines used in this study.

FIG. 3 shows the identification of stable cell types within the diverse microenvironment in mouse lymph nodes based on trancriptomic clusters.

FIG. 4 shows Pearson correlation coefficient of response signatures to a select group of cytokines. These cytokine responses are consistent across biological replicates.

FIGS. 5A-5B. FIG. 5A shows the identification of cytokines that enhance natural killer (NK) cell cytotoxicity relative to a phosphate-buffered saline (PBS) control. FIG. 5B shows the cytotoxic signature scores for the most active cytokines identified in FIG. 5A.

FIG. 6A shows NK cell polarization states in steady state. FIG. 6B shows NK cell polarization states following short-term stimulation with Interleukin-7 receptor (IL7r), Granzyme B (Gzmb), interferon-stimulated gene 15 (Isg15), killer cell lectin-like receptor 4 (Klra4), Srm, and Mcm5.

FIG. 7 shows a reconstruction of dynamic interferon networks.

FIG. 8A shows human PBMC cytokine signatures in CD14 monocytes. Shading represents upregulated genes in the corresponding cytokine stimulation. Each column represents an individual cell, grouped by cytokine treatment. FIG. 8B shows human PBMC cytokine signatures in CD16 monocytes. Shading represents upregulated genes in the corresponding cytokine stimulation. Each column represents an individual cell, grouped by cytokine treatment.

FIG. 9 shows human PBMC cytokine stimulation data in response to IL1B, IL6, IL10, and pICLC. The map shows individual cells as dots, profiled under each stimulation condition.

FIG. 10 shows that IFNa, IFNb, IL15, IL18, IFNk, IL36a, IFNg, Flt3I, OX40L, and GM-CSF are important mediators for vaccination. It is also shown that these mediators exert their effects at different time points.

FIG. 11 shows deconvolution of classical dendritic cell 1 (cDC1) cell type gene expression for 12 time points following stimulation with a vaccine. The shading on the heatmap represents the relative weight of the cytokine in the corresponding time point post vaccination. The results show that IL15, IFNa1, IFNb, TFNa, IL1a, BAFF, IFNg, Flt3I, IL6, IL18, OX40L, IL27, IL36-RA, GM-CSF, and IL34 are important cytokines in vaccine response.

FIG. 12 shows cytokine enrichment results of cDC1 cell type six hours post vaccination.

FIG. 13 shows unbiased identification of similarities and differences between biological features of cytokine responses using reactome pathway enrichment analysis on CD8 T cells.

FIG. 14 shows that many cytokines induce cell-type specific transcriptomic changes. It is shown that IL1b induces distinct transcriptomic changes in dendritic cells (DCs) and macrophages.

FIGS. 15A and B show assessment of the common features and differences of response to IL1a and IL1b. Topic models were used to study gene programs that are up- or down-regulated post cytokine stimulation. Of the 20 topics identified, it was found that 7 of them change in expression post-stimulation, suggesting that these gene programs are related to cell states.

FIG. 16 shows that a global quantification was performed and showed divergent transcriptomic responses across cell types in each cytokine. The results show that different cytokines target distinct cell types, and the vast majority of the cytokines induce changes in at least one cell type.

FIG. 17 shows reconstruction of dynamic cell-cell communication networks under vaccination. Construction of an IL1a cytokine network based on ligand expression, receptor expression, and cytokine signature expression is shown.

FIG. 18A, FIG. 18B, and FIG. 18C show example signatures from combinations of immunomodulating agents.

FIG. 19 shows the identification of macrophage and monocyte states, with example markers and drivers for each state. 21 clusters of macrophage/monocyte states were identified. Cluster 8 is strongly enriched for IL10, representing an anti-inflammatory phenotype. Clusters 4, 12, and 14 are strongly enriched for representing an activated phenotype.

FIG. 20 shows identification of plasmacytoid dendritic cells (pDC) states, with example markers and drivers for each state. 14 clusters of pDC states were identified. Cytokines strongly enriched in each phenotype are identified. Cluster 5 is shown as an example.

FIG. 21 shows identification of gamma delta T cell states, with example markers and drivers for each state. 33 clusters of gdT cell states were identified. Cluster 18 is enriched for cytotoxic molecules. Drivers for this cluster were identified to enhance gdT cell-based therapy. Cluster 11 was identified to be strongly enriched by IL17 family stimulated cells. IL17 cytokines are involved in a variety of autoimmune diseases, so this cluster can be a target for therapeutic intervention.

FIG. 22 shows identification of dendritic cell states, with example markers and drivers for each state. 20 clusters of dendritic cell states were identified. Many have distinct phenotypes from unstimulated samples. Cluster 8 is enriched for molecules that mark an activated DC phenotype (e.g., CD40 expression). Drivers for this state were identified (e.g., TNFα, EGF, G-CSF).

FIG. 23 shows identification of CD8 T cell states, with example markers and drivers for each state. 17 subclusters of CD8 T cells were identified. Many have distinct phenotypes compared to PBS control. Cluster 3 cells are enriched for cytotoxic molecules (e.g., Gzmb) that can enhance CD8 T cells' ability to kill target cells (e.g., cancer cells). Cluster 11 cells are enriched for growth receptors (e.g., ESR), which may enhance their growth. Drivers are identified.

FIG. 24 shows identification of CD4 T cell states, with example markers and drivers for each state. 14 subclusters of CD4 T cells were identified. Many have distinct phenotypes compared to PBS control. Cluster 3 cells are mainly driven by Th2 cytokines (e.g., IL3, IL4), making them more potent for bacterial and parasite infections. Eliminating this cell state can be used as a strategy to reduce allergy symptoms.

FIG. 25 shows identification of B cell states, with example drivers for each state.

FIG. 26A shows reconstruction of cell-cell communication networks in checkpoint blockade immunotherapy. FIG. 26B shows identification of factors enhancing NK cell response in cancer immunotherapy. The CREA-ORA approach was used to deconvolve tumor checkpoint blockade immunotherapy response. The top cytokines in mediating an effective NK cell response (e.g., IL18, IL36α, IL33) are shown. These can be combined with existing cancer therapies to enhance efficacy.

FIG. 27A-27C show generation of a single-cell RNA-seq dictionary of cell type-specific gene expression signatures in response to 86 cytokines. FIG. 27A shows a schematic of experimental and computational workflow. FIG. 27B shows a t-SNE map of all 399,861 cells collected from lymph nodes after cytokine stimulations or without stimulation (PBS controls), shaded by cell type identity. Major cell types are labeled. FIG. 27C provides a heatmap showing the magnitude of response (Euclidean distance) in each cell type 4 hours after the corresponding cytokine stimulation relative to PBS controls. Cytokines investigated and their associated cytokine families are labeled. Families with few members are listed under “Other”.

FIG. 28A-28B show that cytokines induce cell-type-specific transcriptomic responses. FIG. 28A shows the number of differentially expressed genes (false discovery rate (FDR)<0.1 and log₂ fold change >0.3) following each cytokine treatment, grouped by sharing pattern; either uniquely expressed by one cell type (top) or shared by two or more cell types (bottom). The combinations of cell types with the largest number of shared genes (>15 differentially expressed genes (DEGs)) are shown. The maximum number of DEGs in each group are labeled. FIG. 28B shows differentially expressed gene programs (GPs) following (top) IFN-α/β or (bottom) IL-1α/β treatment with respect to PBS controls. Size is proportional to statistical significance, and shade is proportional to effect size. GPs that are significantly different between stimulation and PBS (FDR<0.001 and effect size >1.5) are shown. (Top) GPs enriched for type II interferon stimulated genes are not shown. Top-weighted genes in each GP and representative enriched gene sets (q<0.05; black tiles) for the top-weighted genes in each GP are shown. Hallmark gene sets from the MSigDB database were used.⁴⁶

FIG. 29A-29N show identification of major single cytokine-driven cellular polarization states. A 2-dimensional uniform manifold approximation and projection (UMAP) showing polarization states of each cell type is provided: FIG. 29A, B cell, FIG. 29B, CD4+ T cell, FIG. 29C, CD8 T cell, FIG. 29D, γδ T cell, FIG. 29E, regulatory T cell (Treg), FIG. 29F, NK cell, FIG. 29G, cDC1, FIG. 29H, cDC2, FIG. 29I, migratory dendritic cell (MigDC), FIG. 29J, Langerhans cell, FIG. 29K, pDC, FIG. 29L, Macrophage, FIG. 29M, Monocyte, FIG. 29N, Neutrophil. Cell state name, marker genes, and single cytokine drivers are shown in tables for each cell type. Shading is matched between cells corresponding to the cell state of the same shade. Shading in each panel is independent of other panels. Cytokines in the right column shaded gray are likely secondary inducers.

FIG. 30A-30D show a cytokine production map by cell type. FIG. 30A shows a heatmap of cytokine gene expression by cell type. Shown are expressions of genes corresponding to the 86 cytokines investigated in this study with detectable expression in single cell RNA-sequencing (scRNA-seq). Expression values are normalized relative to the maximum expression in each row. FIG. 30B provides a scatter plot showing the abundance of each cell type in lymph nodes and the number of cytokine genes expressed. Smoothed conditional means and 0.95 confidence intervals are shown. FIG. 30C-30D show fibroblastic reticular cell (FRC) and cDC1 portions of cytokine-mediated cell-cell interactome.

FIG. 31A-31D show that Immune Response Enrichment Analysis (IREA) assesses cytokine responses based on transcriptomic data. FIG. 31A shows a problem statement for cytokine response inference. FIG. 31B illustrates the IREA software input and output. FIG. 31C-31D show IREA analysis on cells collected from tumor microenvironment following anti-PD1 treatment. FIG. 31C provides a compass plot showing IREA score for each of 86 cytokines in NK cells relative to untreated condition. Bar length represents enrichment score, shade represents FDR, with darker shades representing more significant enrichment (dark shading: enriched in anti-PD1 treatment, lighter shading: enriched in untreated control). FIG. 31D shows an inferred cell-cell communication network of cytokines. For ease of identification, cytokines are plotted from left to right in each segment in the same order as the legend.

FIG. 32A-32G show scRNA-seq data summary and quality metrics. FIG. 32A shows distribution of the number of genes detected, number of mRNA transcripts detected, and percentage of mitochondrial gene content after performing quality control in each cytokine-treated or PBS-treated condition. FIG. 32B shows two-dimensional t-SNE visualization of all cells, shaded by any cytokine treatment (light) or PBS treatment control (dark). FIG. 32C shows two-dimensional t-SNE visualization of all cells, shaded by the Louvain clusters identified from global clustering. FIG. 32D shows expression and percentage of cells expressing each cell type marker gene in each global Louvain cluster. FIG. 32E shows expression and percentage of cells expressing each cell type marker gene in each annotated cell type. FIG. 32 F shows the cell type composition in each sample. FIG. 32G shows the change in the cell fraction of CD3− and CD19− immune cells after cytokine treatment relative to PBS-treated samples (*denotes FDR adjusted Wilcoxon rank-sum tests p-value <0.3).

FIG. 33A-33D show additional scRNA-seq data quality metrics. FIG. 33A shows the distribution of pairwise cell-cell Euclidean distance of the entire transcriptome between PBS-treated cells within the same sample processing batches or between different sample processing batches. FIG. 33B shows Pearson correlation coefficients between gene expression signatures obtained from different animal replicates, using cDCs as a representative example. FIG. 33C shows IFN-α1 as a positive control of cytokine effects, showing the impact of the cytokine on the cells profiled using this framework, showing a robust induction of Isg15 expression. FIG. 33D provides a positive control, showing that ISGs can accurately classify IFN-α1 treated cells vs. PBS-treated cells in all treatment conditions. ISGs are obtained from the MSigDB hallmark gene set.

FIG. 34A-34C show cell-type-specific responses to cytokines. FIG. 34A-34B show the top 50 overexpressed genes in any cell type for comparisons between cytokine-treated cells relative to PBS-treated cells of the same cell type. FIG. 34C shows the percentage of differentially expressed genes that are unique to a single cell type (labeled “1” in the legend on the right) or shared by two or more cell types (as labeled in the legend on the right) after cytokine treatment. Same cells as in FIG. 28A with different differential gene expression cutoffs (FC: log 2 fold change) showing consistency of the cell-type-specific effects irrespective of cutoff thresholds.

FIG. 35 shows gene program analysis of IL-18, IL-33, IL-36a, IL-2, IL-4, IL-7, IL-15, IL-3, GM-CSF, and TNF-α. Differentially expressed gene programs following cytokine treatment with respect to PBS controls. Size is proportional to statistical significance and shading is proportional to effect size. Gene programs (GPs) that are significantly different between cytokine treatment and PBS treatment (FDR<0.01 and effect size >1) are shown. Top-weighted genes in each GP. Representative enriched gene sets for the top-weighted genes in each GP.

FIG. 36A-36J show cell state, marker gene expression, and gene program analysis of B cells. FIG. 36A shows cell type polarization states, marker genes, single cytokine drivers, and UMAP visualization of all B cells, shaded by polarization states. FIG. 36B shows UMAP visualization of all B cells, shaded by Louvain subcluster analysis of B cells only. FIG. 36C shows UMAP visualization of B cells, shaded by cytokine treatment (dark) or PBS treatment control (light). FIG. 36D shows the top overexpressed genes in each Louvain subcluster, shaded by row-normalized expression. FIG. 36E shows the relative expression of representative marker genes in cytokine treated cells to PBS-treated cells. FIG. 36F shows the fraction of cells in each subcluster. FIG. 36G shows FDR adjusted p-value of hypergeometric tests showing the over-representation of a subcluster in a sample. Size of the circle represents significance. Black shading indicates p<0.01. FIG. 36H shows gene programs of B cells obtained from non-negative matrix factorization (NMF) analysis. Size of the circle shows FDR adjusted p-value of Wilcoxon rank-sum test between cells from cytokine-treated condition and PBS-treated condition. Shading represents effect size according to the legend provided. FIG. 36I shows the top 20 genes in each gene program identified by NMF. FIG. 36J shows the average gene module weight of each subcluster.

FIG. 37A-37J show cell state, marker gene expression, and gene program analysis of CD4+ T cells. FIG. 37A shows cell type polarization states, marker genes, single cytokine drivers, and UMAP visualization of all CD4+ T cells, shaded by polarization states. FIG. 37B shows UMAP visualization of all CD4+ T cells, shaded by Louvain subcluster analysis of CD4+ T cells only. FIG. 37C shows UMAP visualization of CD4+ T cells, shaded by cytokine treatment (dark) or PBS treatment control (light). FIG. 37D shows the top overexpressed genes in each Louvain subcluster, shaded by row-normalized expression. FIG. 37E shows relative expression of representative marker genes in cytokine treated cells to PBS-treated cells. FIG. 37F shows the fraction of cells in each subcluster. FIG. 37G shows FDR adjusted p-value of hypergeometric tests showing the over-representation of a subcluster in a sample. Size of the circle represents significance. Black shading indicates p<0.01. FIG. 37H shows gene programs of CD4+ T cells obtained from NMF analysis, size of the circle shows FDR adjusted p-value of Wilcoxon rank-sum test between cells from cytokine-treated condition and PBS-treated condition. Shading represents effect size according to the legend provided. FIG. 37I shows the top 20 genes in each gene program identified by NMF. FIG. 37J shows the average gene module weight of each subcluster.

FIG. 38A-38J show cell state, marker gene expression, and gene program analysis of CD8+ T cells. FIG. 38A shows cell type polarization states, marker genes, single cytokine drivers, and UMAP visualization of all CD8+ T cells, shaded by polarization states. FIG. 38B shows UMAP visualization of all CD8+ T cells, shaded by Louvain subcluster analysis of CD8+ T cells only. FIG. 38C shows UMAP visualization of CD8+ T cells, shaded by cytokine treatment (dark) or PBS treatment control (light). FIG. 38D shows the top overexpressed genes in each Louvain subcluster, shaded by row-normalized expression. FIG. 38E shows the relative expression of representative marker genes in cytokine treated cells to PBS-treated cells. FIG. 38F shows the fraction of cells in each subcluster. FIG. 38G shows FDR adjusted p-value of hypergeometric tests showing the over-representation of a subcluster in a sample. Size of the circle represents significance. Black shading indicates p<FIG. 38H shows gene programs of CD8+ T cells obtained from NMF analysis. Size of the circle shows FDR adjusted p-value of Wilcoxon rank-sum test between cells from cytokine-treated condition and PBS-treated condition. Shading represents effect size according to the legend provided. FIG. 38I shows the top 20 genes in each gene program identified by NMF. FIG. 38J shows the average gene module weight of each subcluster.

FIG. 39A-39J show cell state, marker gene expression, and gene program analysis of γδ T cell. FIG. 39A shows cell type polarization states, marker genes, single cytokine drivers, and UMAP visualization of all γδ T cells in the Itgae+(CD103+) cluster, shaded by polarization states. FIG. 39B shows UMAP visualization of all γδ T cells, shaded by Louvain subcluster analysis of γδ T cells only. FIG. 39C shows UMAP visualization of γδ T cells, shaded by cytokine treatment (dark) or PBS treatment control (light). FIG. 39D shows the top overexpressed genes in each Louvain subcluster, shaded by row-normalized expression. FIG. 39E shows the relative expression of representative marker genes in cytokine treated cells to PBS-treated cells. FIG. 39F shows the fraction of cells in each subcluster. FIG. 39G shows FDR adjusted p-value of hypergeometric tests showing the over-representation of a subcluster in a sample. Size of the circle represents significance. Black shading indicates p<FIG. 39H shows gene programs of γδ T cells obtained from NMF analysis. Size of the circle shows FDR adjusted p-value of Wilcoxon rank-sum test between cells from cytokine-treated condition and PBS-treated condition. Shading represents effect size according to the legend provided. FIG. 39I shows the top 20 genes in each gene program identified by NMF. FIG. 39J shows the average gene module weight of each subcluster.

FIG. 40A-40J show cell state, marker gene expression, and gene program analysis of regulatory T cells. FIG. 40A shows cell type polarization states, marker genes, single cytokine drivers, and UMAP visualization of all Treg cells, shaded by polarization states. FIG. 40B shows UMAP visualization of all Treg cells, shaded by Louvain subcluster analysis of Treg cells only. FIG. 40C shows UMAP visualization of Treg cells, shaded by cytokine treatment (dark) or PBS treatment control (light). FIG. 40D shows the top overexpressed genes in each Louvain subcluster, shaded by row-normalized expression. FIG. 40E shows the relative expression of representative marker genes in cytokine treated cells to PBS-treated cells. FIG. 40F shows the fraction of cells in each subcluster. FIG. 40G shows FDR adjusted p-value of hypergeometric tests showing the over-representation of a subcluster in a sample. Size of the circle represents significance. Black shading indicates p<0.01. FIG. 40H shows gene programs of Treg cells obtained from NMF analysis. Size of the circle shows FDR adjusted p-value of Wilcoxon rank-sum test between cells from cytokine-treated condition and PBS-treated condition. Shading represents effect size according to the legend provided. FIG. 40I shows the top 20 genes in each gene program identified by NMF. FIG. 40J shows average gene module weight of each sub cluster.

FIG. 41A-41J show cell state, marker gene expression, and gene program analysis of NK cells. FIG. 41A shows cell type polarization states, marker genes, single cytokine drivers, and UMAP visualization of all NK cells, shaded by polarization states. FIG. 41B shows UMAP visualization of all NK cells, shaded by Louvain subcluster analysis of NK cells only. FIG. 41C shows UMAP visualization of NK cells, shaded by cytokine treatment (dark) or PBS treatment control (light). FIG. 41D shows the top overexpressed genes in each Louvain subcluster, shaded by row-normalized expression. FIG. 41E shows the relative expression of representative marker genes in cytokine treated cells to PBS-treated cells. FIG. 41F shows the fraction of cells in each subcluster. FIG. 41G shows FDR adjusted p-value of hypergeometric tests showing the over-representation of a subcluster in a sample. Size of the circle represents significance. Black shading indicates p<0.01. FIG. 41H shows gene programs of NK cells obtained from NMF analysis. Size of the circle shows FDR adjusted p-value of Wilcoxon rank-sum test between cells from cytokine-treated condition and PBS-treated condition. Shading represents effect size according to the legend provided. FIG. 41I shows the top 20 genes in each gene program identified by NMF. FIG. 41J shows the average gene module weight of each subcluster.

FIG. 42A-42J show cell state, marker gene expression, and gene program analysis of cDC1 cells. FIG. 42A shows cell type polarization states, marker genes, single cytokine drivers, and UMAP visualization of all cDC1 cells in the Itgae+(CD103+) cluster, shaded by polarization states. FIG. 42B shows UMAP visualization of all cDC1 cells, shaded by Louvain subcluster analysis of cDC1 cells only. FIG. 42C shows UMAP visualization of cDC1 cells, shaded by cytokine treatment (dark) or PBS treatment control (light). FIG. 42D shows the top overexpressed genes in each Louvain subcluster, shaded by row-normalized expression. FIG. 42E shows the relative expression of representative marker genes in cytokine treated cells to PBS-treated cells. FIG. 42F shows the fraction of cells in each subcluster. FIG. 42G shows FDR adjusted p-value of hypergeometric tests showing the over-representation of a subcluster in a sample. Size of the circle represents significance. Black shading indicates p<0.01. FIG. 42H shows gene programs of cDC1 cells obtained from NMF analysis. Size of the circle shows FDR adjusted p-value of Wilcoxon rank-sum test between cells from cytokine-treated condition and PBS-treated condition. Shading represents effect size according to the legend provided. FIG. 42I shows the top 20 genes in each gene program identified by NMF. FIG. 42J shows the average gene module weight of each subcluster.

FIG. 43A-43J show cell state, marker gene expression, and gene program analysis of cDC2 cells. FIG. 43A shows cell type polarization states, marker genes, single cytokine drivers, and UMAP visualization of all cDC2 cells, shaded by polarization states. FIG. 43B shows UMAP visualization of all cDC2 cells, shaded by Louvain subcluster analysis of cDC2 cells only. FIG. 43C shows UMAP visualization of cDC2 cells, shaded by cytokine treatment (dark) or PBS treatment control (light). FIG. 43D shows the top overexpressed genes in each Louvain subcluster, shaded by row-normalized expression. FIG. 43E shows the relative expression of representative marker genes in cytokine treated cells to PBS-treated cells. FIG. 43F shows the fraction of cells in each subcluster. FIG. 43G shows FDR adjusted p-value of hypergeometric tests showing the over-representation of a subcluster in a sample. Size of the circle represents significance. Black shading indicates p<0.01. FIG. 43H shows gene programs of cDC2 cells obtained from NMF analysis. Size of the circle shows FDR adjusted p-value of Wilcoxon rank-sum test between cells from cytokine-treated condition and PBS-treated condition. Shading represents effect size according to the legend provided. FIG. 43I shows the top 20 genes in each gene program identified by NMF. FIG. 43J shows the average gene module weight of each subcluster.

FIG. 44A-44J show cell state, marker gene expression, and gene program analysis of migratory DCs. FIG. 44A shows cell type polarization states, marker genes, single cytokine drivers, and UMAP visualization of all MigDC cells in Zeb2-cluster, shaded by polarization states. FIG. 44B shows UMAP visualization of all MigDC cells, shaded by Louvain subcluster analysis of MigDC cells only. FIG. 44C shows UMAP visualization of MigDC cells, shaded by cytokine treatment (dark) or PBS treatment control (light). FIG. 44D shows the top overexpressed genes in each Louvain subcluster, shaded by row-normalized expression. FIG. 44E shows the relative expression of representative marker genes in cytokine treated cells to PBS-treated cells. FIG. 44F shows the fraction of cells in each subcluster. FIG. 44G shows FDR adjusted p-value of hypergeometric tests showing the over-representation of a subcluster in a sample. Size of the circle represents significance. Black shading indicates p<0.01. FIG. 44H shows gene programs of MigDC cells obtained from NMF analysis. Size of the circle shows FDR adjusted p-value of Wilcoxon rank-sum test between cells from cytokine-treated condition and PBS-treated condition. Shading represents effect size according to the legend provided. FIG. 44I shows the top 20 genes in each gene program identified by NMF. FIG. 44J shows the average gene module weight of each subcluster.

FIG. 45A-45J show cell state, marker gene expression, and gene program analysis of Langerhans cells. FIG. 45A shows cell type polarization states, marker genes, single cytokine drivers, and UMAP visualization of all Langerhans cells, shaded by polarization states. FIG. shows UMAP visualization of all Langerhans cells, shaded by Louvain subcluster analysis of Langerhans cells only. FIG. 45C shows UMAP visualization of Langerhans cells, shaded by cytokine treatment (dark) or PBS treatment control (light). FIG. 45D shows the top overexpressed genes in each Louvain subcluster, shaded by row-normalized expression. FIG. shows the relative expression of representative marker genes in cytokine treated cells to PBS-treated cells. FIG. 45F shows the fraction of cells in each subcluster. FIG. 45G shows FDR adjusted p-value of hypergeometric tests showing the over-representation of a subcluster in a sample. Size of the circle represents significance. Black shading indicates p<FIG. 45H shows gene programs of Langerhans cells obtained from NMF analysis. Size of the circle shows FDR adjusted p-value of Wilcoxon rank-sum test between cells from cytokine-treated condition and PBS-treated condition. Shading represents effect size according to the legend provided. FIG. 45I shows the top 20 genes in each gene program identified by NMF. FIG. 45J shows the average gene module weight of each subcluster.

FIG. 46A-46J shows cell state, marker gene expression, and gene program analysis of plasmacytoid DCs. FIG. 46A shows cell type polarization states, marker genes, single cytokine drivers, and UMAP visualization of all pDC cells, shaded by polarization states. FIG. 46B shows UMAP visualization of all pDC cells, shaded by Louvain subcluster analysis of pDC cells only. FIG. 46C shows UMAP visualization of pDC cells, shaded by cytokine treatment (dark) or PBS treatment control (light). FIG. 46D shows the top overexpressed genes in each Louvain subcluster, shaded by row-normalized expression. FIG. 46E shows the relative expression of representative marker genes in cytokine treated cells to PBS-treated cells. FIG. 46F shows the fraction of cells in each subcluster. FIG. 46G shows FDR adjusted p-value of hypergeometric tests showing the over-representation of a subcluster in a sample. Size of the circle represents significance. Black shading indicates p<0.01. FIG. 46H shows gene programs of pDC cells obtained from NMF analysis. Size of the circle shows FDR adjusted p-value of Wilcoxon rank-sum test between cells from cytokine-treated condition and PBS-treated condition. Shading represents effect size according to the legend provided. FIG. 46I shows the top 20 genes in each gene program identified by NMF. FIG. 46J shows the average gene module weight of each subcluster.

FIG. 47A-47J show cell state, marker gene expression, and gene program analysis of macrophages (Marco+cluster). FIG. 47A shows cell type polarization states, marker genes, single cytokine drivers, and UMAP visualization of all macrophage cells, shaded by polarization states. FIG. 47B shows UMAP visualization of all macrophage cells, shaded by Louvain subcluster analysis of macrophage cells only. FIG. 47C shows UMAP visualization of macrophage cells, shaded by cytokine treatment (dark) or PBS treatment control (light). FIG. 47D shows the top overexpressed genes in each Louvain subcluster, shaded by row-normalized expression. FIG. 47E shows the relative expression of representative marker genes in cytokine treated cells to PBS-treated cells. FIG. 47F shows the fraction of cells in each subcluster. FIG. 47G shows FDR adjusted p-value of hypergeometric tests showing the over-representation of a subcluster in a sample. Size of the circle represents significance. Black shading indicates p<0.01. FIG. 47H shows gene programs of macrophage cells obtained from NMF analysis. Size of the circle shows FDR adjusted p-value of Wilcoxon rank-sum test between cells from cytokine-treated condition and PBS-treated condition. Shading represents effect size according to the legend provided. FIG. 47I shows the top 20 genes in each gene program identified by NMF. FIG. 47J shows the average gene module weight of each subcluster.

FIG. 48A-48J show cell state, marker gene expression, and gene program analysis of monocytes. FIG. 48A shows cell type polarization states, marker genes, single cytokine drivers, and UMAP visualization of all monocyte cells, shaded by polarization states. FIG. 48B shows UMAP visualization of all monocyte cells, shaded by Louvain subcluster analysis of monocyte cells only. FIG. 48C shows UMAP visualization of monocyte cells, shaded by cytokine treatment (dark) or PBS treatment control (light). FIG. 48D shows the top overexpressed genes in each Louvain subcluster, shaded by row-normalized expression. FIG. 48E shows the relative expression of representative marker genes in cytokine treated cells to PBS-treated cells. FIG. 48F shows the fraction of cells in each subcluster. FIG. 48G shows FDR adjusted p-value of hypergeometric tests showing the over-representation of a subcluster in a sample. Size of the circle represents significance. Black shading indicates p<FIG. 48H shows gene programs of monocyte cells obtained from NMF analysis. Size of the circle shows FDR adjusted p-value of Wilcoxon rank-sum test between cells from cytokine-treated condition and PBS-treated condition. Shading represents effect size according to the legend provided. FIG. 48I shows the top 20 genes in each gene program identified by NMF. FIG. 48J shows average gene module weight of each subcluster.

FIG. 49A-49J show cell state, marker gene expression, and gene program analysis of neutrophils. FIG. 49A shows cell type polarization states, marker genes, single cytokine drivers, and UMAP visualization of all neutrophil cells, shaded by polarization states. FIG. 49B shows UMAP visualization of all neutrophil cells, shaded by Louvain subcluster analysis of neutrophil cells only. FIG. 49C shows UMAP visualization of neutrophil cells, shaded by cytokine treatment (dark) or PBS treatment control (light). FIG. 49D shows the top overexpressed genes in each Louvain subcluster, shaded by row-normalized expression. FIG. 49E shows the relative expression of representative marker genes in cytokine treated cells to PBS-treated cells. FIG. 49F shows the fraction of cells in each subcluster. FIG. 49G shows FDR adjusted p-value of hypergeometric tests showing the over-representation of a subcluster in a sample. Size of the circle represents significance. Black shading indicates p<FIG. 49H shows gene programs of neutrophil cells obtained from NMF analysis. Size of the circle shows FDR adjusted p-value of Wilcoxon rank-sum test between cells from cytokine-treated condition and PBS-treated condition. Shading represents effect size according to the legend provided. FIG. 49I shows the top 20 genes in each gene program identified by NMF. FIG. 49J shows the average gene module weight of each subcluster.

FIG. 50A-50C show a map of cytokine receptor expression by cell type and additional information on the cytokine production map. FIG. 50A shows the correlation between cell type abundance and the number of distinct cytokines expressed; added B cells and T cells to FIG. 30B (B and T cell abundance is obtained from literature, all other cell types are obtained from PBS-treated conditions in this dictionary). FIG. 50B shows a robustness analysis for FIG. 30B; showing correlations between the numbers of distinct cytokine genes expressed in a cell type and the abundance of the cell type under a variety of cutoff thresholds for a cytokine gene to be considered expressed. FIG. 50C shows a map of cytokine receptor expression by cell type.

FIG. 51A-51B show a draft network of cytokine-mediated cell-cell interactome. FIG. 51A shows an interactome network showing cell-cell communication potential based on cytokine expression and the impact of cytokine on each cell type. FIG. 51B shows the interactome plotted separately by source node for ease of visualization. Source nodes or cells secreting cytokines, cytokines mediating the communication, and sink nodes or cells responding to the cytokines are shown according to the legend provided. An edge is drawn if the connecting cell type produces the cytokine or significantly (FDR<0.25) responds to the cytokine.

FIG. 52 shows cell type properties in cell-cell interactome. Properties of FIG. 51A-51B. For each cell type (a circle), showing the number of cytokines expressed and the number of cell types targeted through any cytokine. Size of the circle is proportional to the total number of edges in the cell-cell interactome.

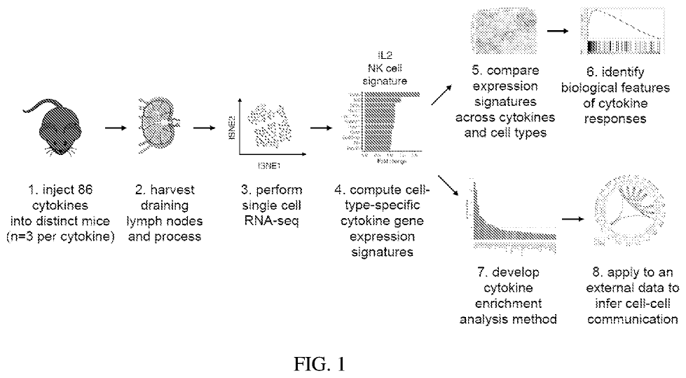

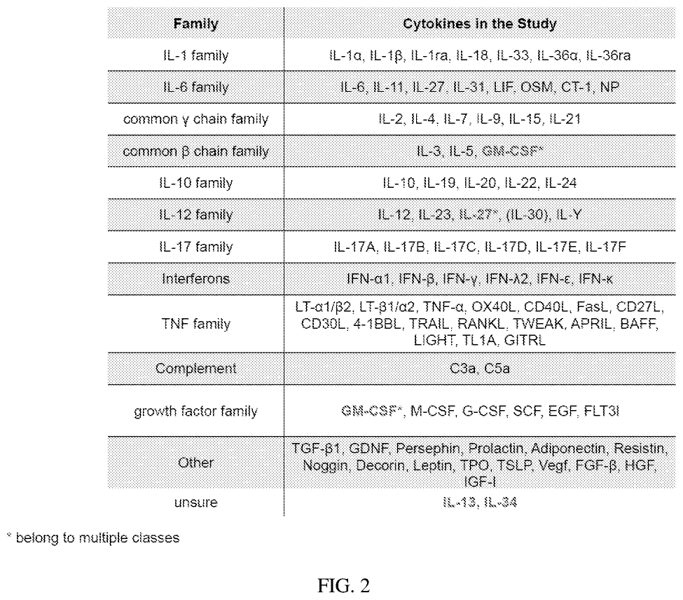

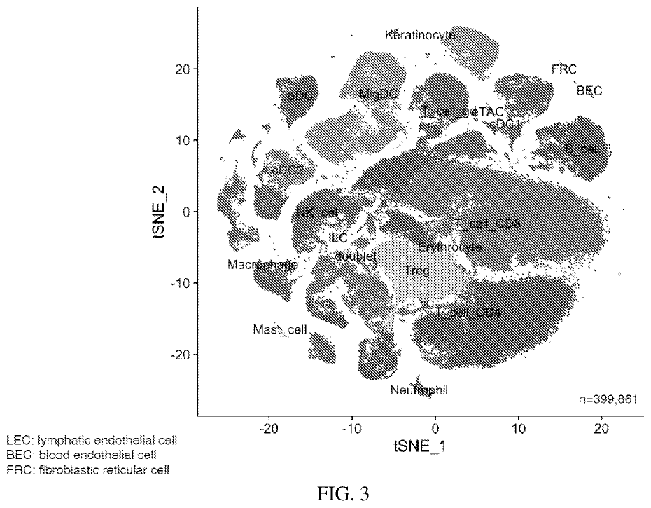

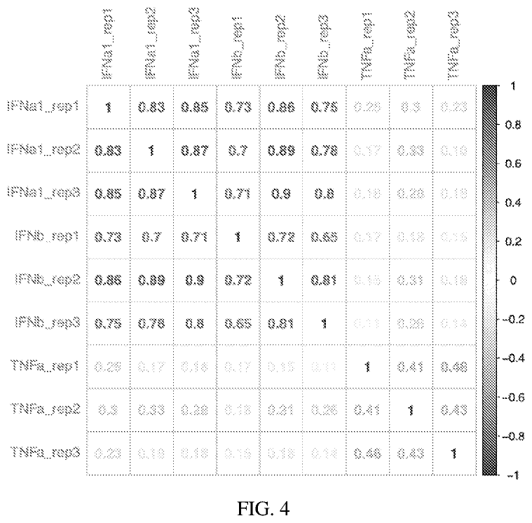

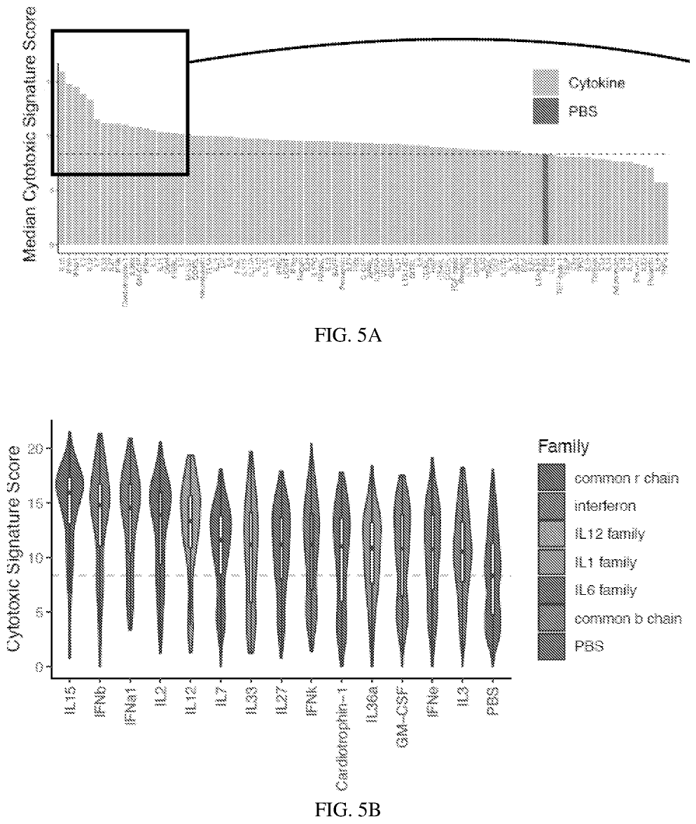

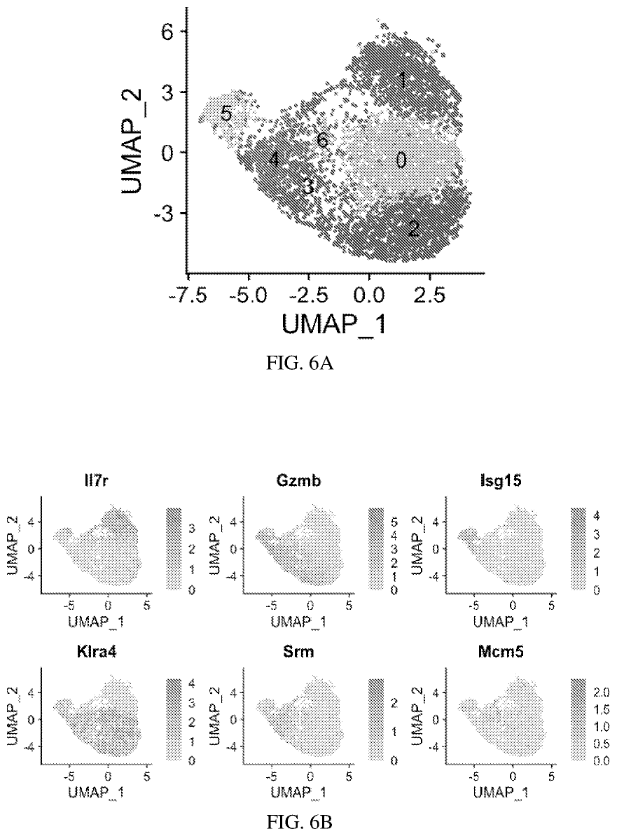

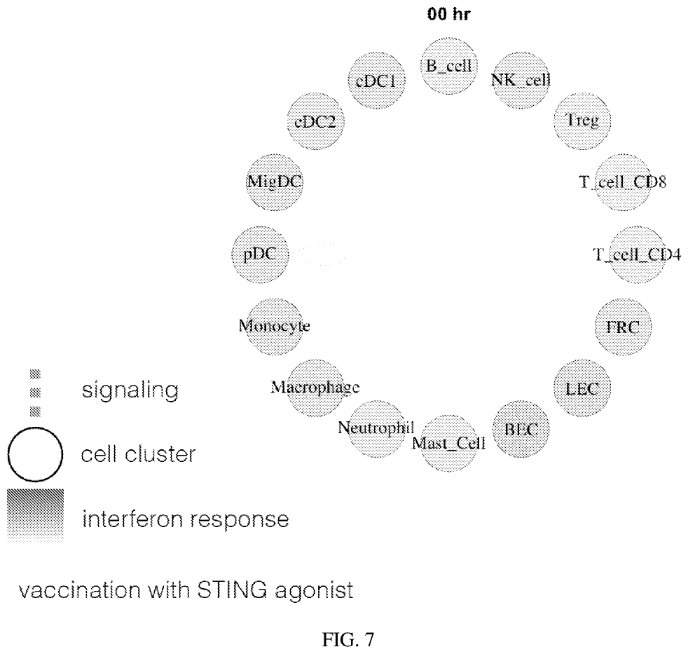

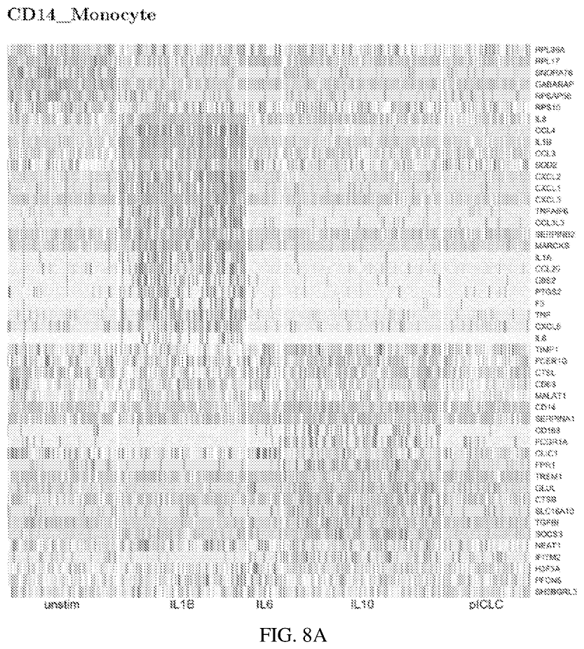

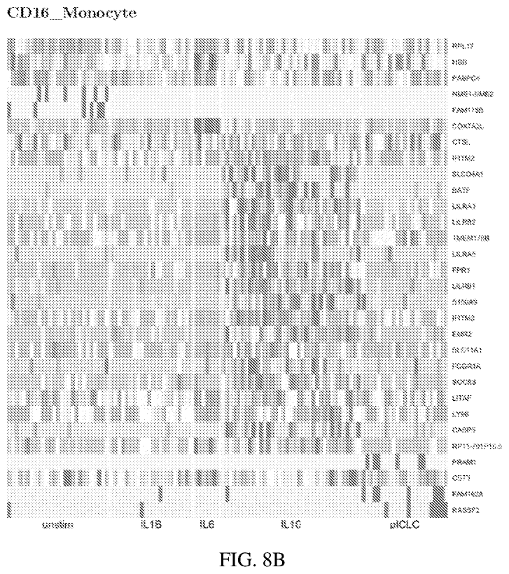

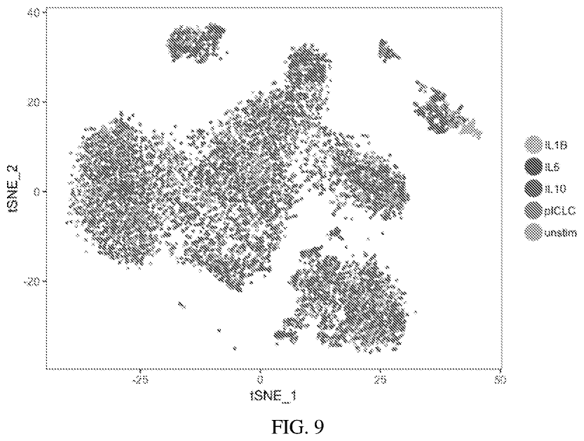

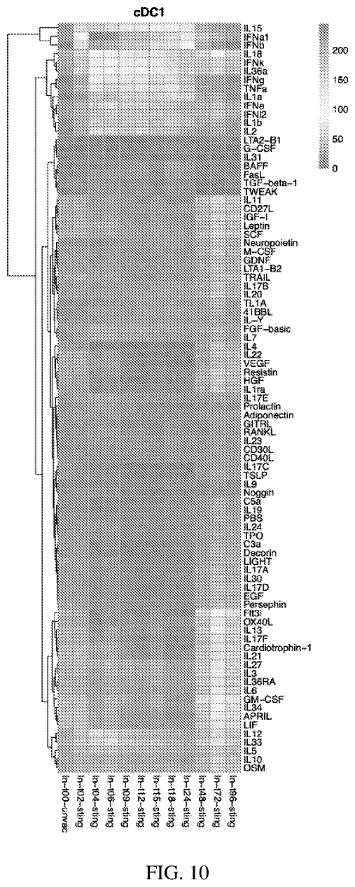

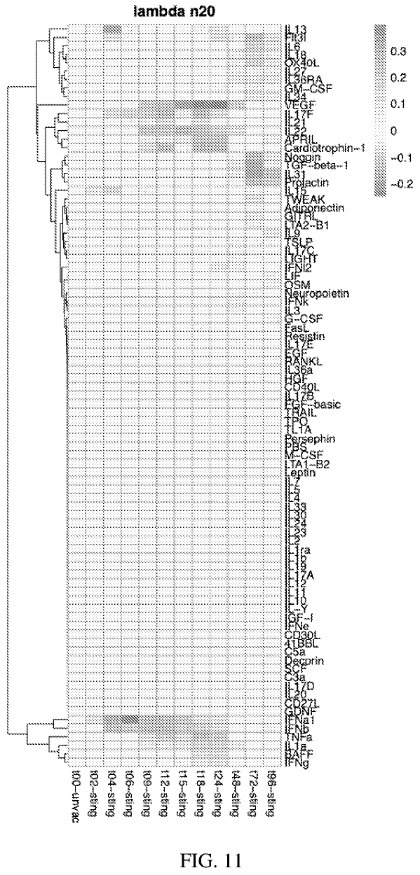

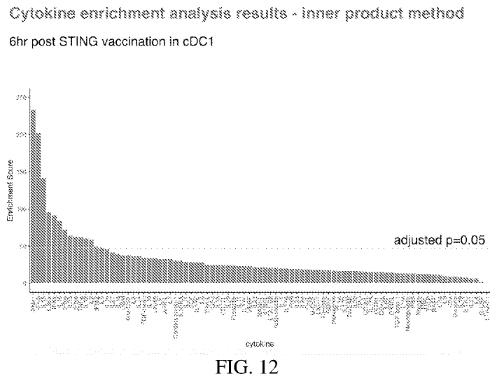

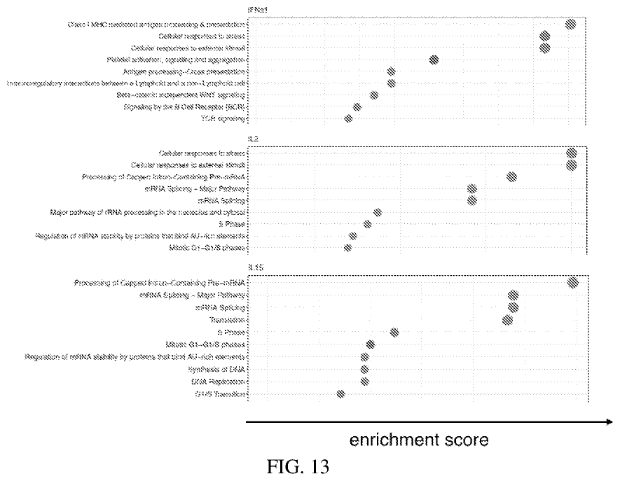

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, an “immune response” is a reaction within an organism (e.g., a human) to defend against foreign invaders such as bacteria, viruses, parasties, and fungi. An immune response can also be a reaction to the presence of cancer cells or a tumor. Immune responses include the innate immune response and the adaptive immune response. The innate immune response is a fast, non-specific response to any sort of pathogen, cancer cell, or other foreign invader. Components of the innate immune response include the skin, mucous membranes, neutrophils, macrophages, and monocytes. The adaptive immune response is a response that is developed by an organism to a specific pathogen or foreign invader. Components of the adaptive immune response include antibodies, dendritic cells, T cells, and B cells. A “signature” of an immune response refers to a measurable difference in a cell-population of cells, tissue, or organism that occurs when the cell, cells, tissue, or organism is treated with an immunomodulating agent. Signatures of an immune response may comprise changes in gene expression (e.g., upregulation or downregulation of the expression of a specific gene or group of genes in response to an immunomodulating agent or other immune stimulus). A signature of an immune response may also be a change in protein expression or in chromatin accessibility. As used herein, the “polarization state” of an immune cell refers to the adoption of a distinct program or specialized function in an immune cell in response to a stimulus (e.g., an immunomodulating agent).

As used herein, an “immunomodulating agent” is any agent (e.g., nucleic acid, protein, small molecule, etc.) that has an effect on an immune response. Immunomodulating agents can either activate or suppress an immune response. Immunomodulating agents include, but are not limited to, cytokines, growth factors, hormones, immune receptor agonists, and vaccine adjuvants. Cytokines are small proteins (approximately 5-20 kDa) that play important roles in cell signaling. Cytokines include chemokines, interferons, interleukins, lymphokines, and tumor necrosis factors (TNFs). Cytokines are produced by a broad range of immune cells, including macrophages, B lymphocytes, T lymphocytes, mast cells, endothelial cells, fibroblasts, and stromal cells. Cytokines act as cell surface receptors. Cytokines include IL-1 family cytokines, IL-6 family cytokines, IL-10 family cytokines, IL-12 family cytokines, IL-17 family cytokines, common γ chain family cytokines, common β chain family cytokines, interferons, and TNF family cytokines. Examples of cytokines and other immunomodulating agents include, but are not limited to, IL1a, IL1b, IL1ra, IL18, IL33, IL36a, IL36ra, IL6, IL11, IL27, IL31, LIF, OSM, CT-1, NP, IL2, IL4, IL7, IL9, IL18, IL15, IL21, IL3, IL5, GM-CSF, IL10, IL19, IL20, IL22, IL24, IL12, IL23, IL27, IL30, IL-Y, IL17A, IL17B, IL17C, IL17D, IL17E, IL17F, IFN-α1, IFN-b, IFN-γ, IFN-λ2, IFN-ε, IFN-k, LT-a1/b2, LT-b1/a2, TNF-α, OX40L, CD40L, FasL, CD27L, CD30L, 4-1BBL, TRAIL, RANKL, TWEAK, APRIL, BAFF, LIGHT, TL1A, GITRL, C3a, C5a, M-CSF, G-CSF, SCF, EGF, FLT3I, TGF-b1, GDNF, Persephin, Prolactin, Adiponectin, Resistin, Noggin, Decorin, Leptin, TPO, TSLP, Vegf, FGF-b, HGF, IGF-I, IL13, IL34, GM-CSF, M-CSF, G-CSF, SCF, EGF, and FLT3.

As used herein, “immune cells” include all types of cells involved in the immune system. Certain immune cells are capable of producing various immunomodulating agents as described above. Immune cell types include, but are not limited to, monocytes, NK cells, T cells (e.g., CD4 T cells, CD8 T cells, regulatory T cells, or gamma delta (gd) T cells), macrophages, lymphatic cells, innate lymphoid cells, neutrophils, blood endothelial cells, fibroblastic reticular cells, mast cells, basophils, Langerhans cells, and dendritic cells (e.g., conventional type 1 dendritic cells, conventional type 2 dendritic cells, migratory dendritic cells, or plasmacytoid dendritic cells). Immune cell types also include B cells, cDC1 cells, cDC2 cells, eTAC cells, ILC cells, LEC cells, pDC cells, migratory T cells. Immune cells may be cells from an animal (e.g., a mammal). Immune cells may be cells from a mouse. Immune cells may be human cells. Immune cells may be grown in vitro or harvested in vivo. Immune cells may be harvested from various sites (e.g., draining lymph node cells, blood, lymph, etc.).

The “immune system,” as used herein, refers to a network of biological processes that protects an organism from diseases. The immune system can detect and respond to a variety of stimuli, such as pathogens, viruses, cancer cells, and inflammatory stimuli. The immune system includes the innate immune system (providing a preconfigured response in an organism) and the adaptive immune system (providing a tailored response to each stimulus by learning to recognize stimuli it has previously encountered). An “immune response” refers to the reaction of the immune system to a stimuli.

As used herein, “gene expression profiling” refers to the measurement of the expression of up to thousands of genes at once to create a global picture of cellular function. Gene expression profiling can be accomplished by performing single-cell RNA sequencing. Performing single-cell RNA sequencing provides the expression profiles of individual cells, allowing patterns of gene expression to be identified through gene clustering analyses. Methods for single-cell RNA sequencing include isolating single cells and their RNA, followed by reverse transcription, amplification, library generation, and sequencing. In some embodiments, individual cells are separated into separate wells. In some embodiments, individual cells are encapsulated in droplets in a microfluidic device, wherein each droplet carries a unique gene-specific identifier sequence, allowing nucleic acids from various cells to be mixed together for sequencing and transcripts from individual cells identified afterward. Gene expression profiling may also be done directly in intact tissue sections using methods of “spatial transcriptomics,” in which RNA is collected from individual cells for reverse transcription, amplification, library generation, and sequencing.

Gene expression profiling may comprise “bulk gene expression profiling.” As used herein, bulk gene expression profiling refers to profiling gene expression (e.g., RNA sequencing data) on multiple cells at the same time (e.g., in complex tissues). Gene expression profiling may also comprise “protein expression profiling.” As used herein, protein expression profiling refers to the identification of proteins expressed in a particular group of cells or tissue (for example, a lymph node) under a particular set of conditions (e.g., after contact with an immunomodulating agent). Gene expression profiling may also refer to “chromatin accessibility profiling.” As used herein, chromatin accessibility profiling refers to the analysis of which regions of a nucleic acid sequence are or are not accessible for factor binding (e.g., for the regulation of gene expression) under a particular set of conditions (e.g., after contact with an immunomodulating agent).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably. An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases. The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990.

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney) or be systemic (e.g., systemic lupus or erythematosus). The treatment of autoimmune diseases is typically with immunosuppression, e.g., medications which decrease the immune response.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

The present disclosure provides methods for determining signatures of immune responses to immunomodulating agents in cells and in tissues. Also provided herein are computational methods for deconvoluting gene expression profiling data from cells and tissues. Further provided herein are methods for treating a disease or disorder (e.g., proliferative diseases such as cancer, autoimmune diseases such as rheumatoid arthritis and psoriasis, allergies, etc.) in a subject comprising administering one or more immunomodulating agents to drive an immune response, in combination with a treatment for the disease or disorder (e.g., an anti-cancer therapy, an anti-viral therapy, a vaccination, etc.).

### Methods for Determining Signatures of Immune Responses

Disclosed herein are methods for determining signatures of immune responses to immunomodulating agents. In some embodiments, the methods comprise the steps of:
(a) administering an immunomodulating agent to a population of cells, wherein the population of immune cells comprises multiple cell types;
(b) harvesting the population of cells;
(c) profiling gene expression in each cell within the population of cells;
(d) correlating cell type and gene expression to compute cell-type-specific gene expression signatures;
(e) repeating steps (a)-(d) for additional immunomodulating agents; and
(f) comparing the cell-type-specific gene expression signatures across immunomodulating agents.

In some embodiments, step (d) further comprises calculating expression signatures associated with each immune-modulating agent relative to a control population of immune cells that has not been administered an immunomodulating agent.

In some embodiments, the immunomodulating agent is a cytokine. In certain embodiments, the cytokine is an IL-1 family cytokine, an IL-6 family cytokine, an IL-10 family cytokine, an IL-12 family cytokine, an IL-17 family cytokine, a common γ chain family cytokine, a common β chain family cytokine, an interferon, or a TNF family cytokine. In some embodiments, the immunomodulating agent is IL1a, IL1b, IL1ra, IL18, IL33, IL36a, IL36ra, IL6, IL11, IL27, IL31, LIF, OSM, CT-1, NP, IL2, IL4, IL7, IL9, IL18, IL15, IL21, IL3, IL5, GM-CSF, IL10, IL19, IL20, IL22, IL24, IL12, IL23, IL27, IL30, IL-Y, IL17A, IL17B, IL17C, IL17D, IL17E, IL17F, IFN-α1, IFN-b, IFN-γ, IFN-λ2, IFN-c, IFN-k, LT-a1/b2, LT-b1/a2, TNF-a, OX40L, CD40L, FasL, CD27L, CD30L, 4-1BBL, TRAIL, RANKL, TWEAK, APRIL, BAFF, LIGHT, TL1A, GITRL, C3a, C5a, M-CSF, G-CSF, SCF, EGF, FLT3I, TGF-b1, GDNF, Persephin, Prolactin, Adiponectin, Resistin, Noggin, Decorin, Leptin, TPO, TSLP, Vegf, FGF-b, HGF, IGF-I, IL13, or IL34, or a combination thereof.

In some embodiments, the immunomodulating agent is a growth factor. Growth factors include, but are not limited to, GM-CSF, M-CSF, G-CSF, SCF, EGF, and FLT3I. In some embodiments, the immunomodulating agent is a hormone. Hormones include, but are not limited to, steroids, eicosanoids, and amino acid or protein derivatives. In some embodiments, the immunomodulating agent is a vaccine adjuvant.

In some embodiments, the population of cells is a population of human cells. In some embodiments, the population of cells is a population of mouse cells, rat cells, or cells from another animal. In certain embodiments, the population of cells comprises at least one cell type. In some embodiments, the population of cells comprises more than one cell type. In certain embodiments, the population of cells comprises at least 2, at least 3, at least 4, or at least 5 different cell types. In some embodiments, the cells are monocytes. In some embodiments, the cell are NK cells. In some embodiments, the cells are T cells (e.g., CD4 T cells, CD8 T cells, regulatory T cells, or gamma delta T cells). In some embodiments, the cells are B cells. In some embodiments, the cells are macrophages. In some embodiments, the cells are lymphatic cells. In some embodiments, the cells are innate lymphoid cells. In some embodiments, the cells are neutrophils. In some embodiments, the cells are blood endothelial cells. In some embodiments, the cells are fibroblastic reticular cells. In some embodiments, the cells are mast cells. In some embodiments, the cells are basophils. In some embodiments, the cells are Langerhans cells. In some embodiments, the cells are dendritic cells (e.g., conventional type 1 dendritic cells, conventional type 2 dendritic cells, migratory dendritic cells, or plasmacytoid dendritic cells).

In certain embodiments, the population of immune cells is in vivo. In certain embodiments, the population of immune cells is in a mammal. In some embodiments, the population of immune cells is within a mouse, a rat, a primate, or another animal. In some embodiments, the population of immune cells is within a human. In some embodiments, the step of harvesting the population of immune cells comprises harvesting and processing cells from draining lymph nodes. In some embodiments, the step of harvesting the population of immune cells comprises harvesting and processing endothelial cells. In some embodiments, the step of harvesting the cells is performed at least 5 minutes, at least 10 minutes, at least 30 minutes, at least 1 hour, at least 2 hours, at least 3 hours, at least 4 hours, at least 5 hours, at least 6 hours, at least 7 hours, at least 8 hours, or at least 12 hours after the step of administering. In certain embodiments, the step of harvesting the cells is performed 4 hours after the step of administering.

In certain embodiments, the step of profiling gene expression comprises performing single-cell RNA sequencing. In some embodiments, the step of profiling gene expression comprises bulk gene expression profiling. In some embodiments, the step of profiling gene expression comprises single-cell gene expression profiling. In some embodiments, the step of profiling gene expression comprises protein expression profiling. In some embodiments, the step of profiling gene expression comprises chromatin accessibility profiling.

In certain embodiments, the method further comprises identifying polarization states of cell types within the population of immune cells in response to immunomodulating agents. In certain embodiments, the method further comprises applying computational methods to compare the cell-type-specific gene expression signatures of an immune response with the cell-type-specific gene expression signatures of an immune response to various other immunomodulating agents obtained using the methods described herein. Computational methods may include determining a target gene expression profile (e.g., the differential profile between post-treatment and pre-treatment with an immunomodulating agent), and determining, using at least some of the signatures of an immune response, at least one immunomodulating agent associated the target gene expression profile. In some embodiments, the at least one immunomodulating agent associated with the target gene expression profile is performed by regressing at least some of the signatures of immune response against the target gene expression profile using a regression technique (e.g., a regularized linear regression technique such as a ridge regression technique, a least absolute shrinkage and selection operator (LASSO regression technique, or an elastic net regression technique). In some embodiments, determining at least one immunomodulating agent associated with the target gene expression profile is performed by correlating at least one signature of immune response with the target expression profile. In some embodiments, determining the at least one immunomodulating agent associated with the target gene expression profile is performed using a Wilcoxon rank-sum test. In some embodiments, the data obtained using the methods described herein may be used to treat or prevent a disease.

In some aspects, the methods described herein are useful for treating any disease related to the immune system, or any disease or condition involving an immune response. In some embodiments, the disease is caused by chronic inflammatory damage (e.g., cancer caused by chronic inflammation). In some aspects, the methods described herein are useful for detecting markers of rejection in advance of an organ transplant. In some aspects, the methods described herein are useful for identifying patients to diagnose an autoimmune disease.

### Methods for Manipulating an Immune Response to Treat a Disease

Also disclosed herein are methods for treating a disease or disorder in a subject in need thereof. In some embodiments, the method comprises administering one or more immunomodulating agents that drive an immune response within the subject in combination with a treatment for the disease or disorder, wherein the immune response is associated with increased efficacy of the treatment. The method can be used to diagnose, prevent, and/or treat any disease or disorder, and in particular, any immune-related disease or disorder. Diseases and disorder include, but are not limited to, proliferative diseases (e.g., cancer), infectious diseases, inflammatory diseases (e.g., allergies), autoimmune diseases, or any other immune-related disease.

In some embodiments, a proliferative disease is cancer. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CIVIL), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosus, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

In some embodiments, the immune response is induced in NK cells. In some embodiments, the immune response comprises enhancing NK cell proliferation to expand the population of NK cells. In some embodiments, the immunomodulating agent is a cytokine. In some embodiments, the immunomodulating agent is selected from the group consisting of IL18, IL15, IFNb, IL2, IL12, IL7, IL33, IL27, IFNk, Cardiotrophin-1, IL36a, GM-CSF, IFNe, IL3, IL11, OSM, 41BBL, IL9, M-CSF, CD40L, Neuropoietin, TL1A, IL21, LIF, IL4, FasL, IL17F, IL17A, IL10, IL17E, IL5, IFNI2, LIGHT, IFNg, Noggin, IL24, IL17C RANKL IL13, BAFF, Persephin, IL23, Flt3I, G-CSF, APRIL, IL36RA, VEGF, GDNF, IL31, LTA1-B2, GITRL, IL6, CD30L, HGF, OX40L, CD27L, FGF-basic, Resistin, IL17B, Leptin, IL30, TRAIL, SCF, IL17D, IL-Y, IGF-I, EGF, IL20, and LTA2-B1. In certain embodiments, the immunomodulating agent is IL18. In certain embodiments, the immunomodulating agent is immune cell receptor agonist (e.g., TLR agonist, NLR agonist, RLR agonist, or STING agonist, or a combination thereof).

In some embodiments, the immune response is induced in dendritic cells. In certain embodiments, the immune response comprises induction of Ccr7, MHC-II, Cd80, Cd83, Cd86, or Mfge8 expression. In some embodiments, the immune response is induced in macrophages. In some embodiments, the immune response comprises induction of iNos in M1-like macrophages. In some embodiments, the immune response comprises induction of MHC-II expression in activated macrophages. In some embodiments, the immune response comprises elevated expression of Arg1 in M2-like macrophages. In some embodiments, the immune response comprises elevated expression of IL10 in M2-like macrophages. In certain embodiments, the immune response comprises decreased expression of M1-like macrophages. In some embodiments, the immune response comprises decreased expression of M2-like macrophages. In some embodiments, the immune response comprises increased CD40 expression. In some embodiments, the immune response comprises increased CD80, CD86, or CD83 expression. In some embodiments, the immune response comprises increased EGFR expression. In some embodiments, the immune response comprises increased or decreased expression of genes that control cell proliferation and/or cell growth.

In some embodiments the autoimmune disease is rheumatoid arthritis. In some embodiments, the immune response comprises decreased antigen presentation molecules on B cells. In some embodiments, the immune response comprises reduction of TNF expression.

In some embodiments, the autoimmune disease is psoriasis. In certain embodiments, the immune response comprises reduction of IL23 expression.

In some embodiments, the disease or disorder is an allergic reaction. In certain embodiments, the immune response comprises decreased expression of IL4, IL5, IL13, or IL33. In certain embodiments, the immune response comprises decreased expression of IL13. In some embodiments, the immune response comprises decreased activation of mast cells. In some embodiments, the immune response comprises decreased activation of basophils. Thus, treatment for an allergic reaction may comprise administering IL4, IL5, IL13, or IL33, or stimulating activation of mast cells or basophils, or any combination thereof.

In some embodiments, the immune response comprises induction of Granzyme B (GZMB). In some embodiments, Granzyme B is induced in T cells. In some embodiments, Granzyme B is induced in NK cells. In some embodiments, the immune response comprises suppression of transforming growth factor beta (TGF-β).

In some embodiments, the treatment is an anti-tumor therapy. In some embodiments, the treatment is an anti-viral therapy. In some embodiments, the treatment is checkpoint blockade therapy. In some embodiments, the treatment is a vaccine. In some embodiments, the immunomodulating agent is a vaccine. In certain embodiments, the immune response comprises regulation of IFNa, IFNb, IL15, IL18, IFNk, IL36α, IFNg, Flt3I, OX40L, or GM-CSF.

In some embodiments, treatment of a disease or disorder (e.g., a viral infection such as COVID-19) comprises depletion of basophils. For example, the results and data disclosed herein shows that production of IL6 occurs at higher levels in basophils. Cell types producing IL6 may be involved cytokine storms, such as in COVID-19 patients. Thus, depletion of basophils may be useful as a COVID-19 treatment, or as a treatment for any disease associated with a cytokine storm (including other viral infections, e.g., infection with influenza viruses or other SARS viruses).

In some embodiments, the disease or disorder being treated is cancer. In some embodiments, treatment for cancer includes administering an immunomodulating agent (e.g., IL12, Cd40LG, IL15, or IL12b). In some embodiments, the immunomodulating agent comprises Flt3 ligand (e.g., in cancer immunotherapy).

In some embodiments, the disease or disorder is angiogenesis. In some embodiments, the immunomodulating agent administered to treat a disease or disorder comprises VEGFC (e.g., in the treatment of angiogenesis).

Further disclosed herein are methods for polarizing a population of cells comprising stimulating the cells with IL7r, Gzmb, Isg15, Klra4, Srm, or Mcm5. In some embodiments, the method comprises polarizing NK cells. Also disclosed herein are methods for expanding a population of cells comprising stimulating the cells with an immunomodulating agent. In some embodiments, the cells are NK cells and the immunomodulating agent is a cytokine. Further disclosed herein are methods of treating cancer comprising administering IL18 to a subject in combination with a cancer therapy, or comprising administering an anti-cancer therapy in combination with inducing GZMN expression in a subject. Methods for treating a viral infection comprising administering an anti-viral treatment in combination with inducing GZMB expression in a subject are also disclosed. Finally, a method of vaccination is disclosed, the method comprising administering a vaccine and stimulating IFNa, IFNb, TFNa, IL15, IL18, IL1a, IFNk, IL36a, IFNg, Flt3I, OX40L, BAFF, IFNg, Flt3I, IL6, IL27, IL36-RA, IL34, or GM-CSF in a subject in need thereof.

### EXAMPLES

### Example 1

scRNA-Seq Profiling of Draining Lymph Node Cells after Cytokine Injection

Single cell RNA-seq data was collected on all lymph node cells in response to exposure to 86 cytokines in vivo. A group of 86 cytokines was selected, with most of the cytokines within each major family (i.e., common β chain (GM-CSF/IL3/IL5), common γ chain (IL-2/4/7/9/15/21), IL1 (7 members), IL6 (5 members), IL10 (5 members), TNF (16 members), IL17 (6 members), IL12 (3 members), interferons (6 members), and complement (2 members)). In addition, a small number of representative cytokines was selected from other protein families with less well-characterized immune functions (e.g., prolactin, leptin). A table showing a list of gene expression signatures induced by each cytokine in each cell type is included in Appendix A. Each freshly reconstituted carrier-free cytokine was injected under the skin in the abdominal flank of distinct mice (with 3 replicate wild type C57BL/6 mice per cytokine) at known bioactive concentrations based on prior literature on individual cytokines. The draining lymph nodes were then harvested 4 hours post injection; the time point was chosen based on prior studies to ensure adequate time for the majority of the transcriptome to respond post-stimulation, but to be early enough to minimize secondary effects that are not specific to the ligand. The lymph node cells were processed using an optimized protocol for viable cell recovery and sample multiplexing. A total of 399,861 cells passed quality control.

The cells were partitioned into clusters and a cell identity was assigned to each cluster based on genes uniquely expressed by the cluster. Preliminarily, 17 cell populations were identified, including CD4 T, CD8 T, T regs, gdT, NK cells, innate lymphoid cells (ILCs), cDC1, cDC2, migratory DC, Langerhans cells, pDC, macrophages, monocytes, lymphatic cells, neutrophils, blood endothelial cells, fibroblastic reticular cells, mast cells, and basophils.

### Cytokines Induce Cell-Type-Specific Gene Expression Changes

Cytokines do not impact all cell types equally. To obtain a map of which cell type is influenced by which cytokine, the number of genes that show differential gene expression between cytokine-treated and untreated mice in each cell type was computed. It was shown that cytokine signatures are highly reproducible across biological replicates. A global quantification was performed and showed divergent transcriptomic responses across cell types in each cytokine (FIG. 16). The results show that different cytokines target distinct cell types, and a majority of the cytokines induce changes in at least one cell type.

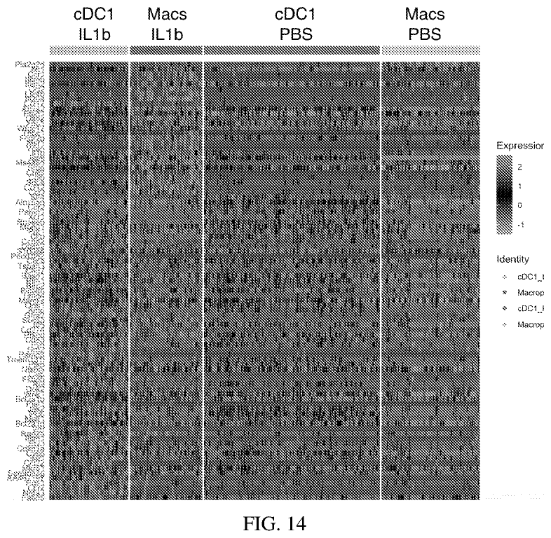

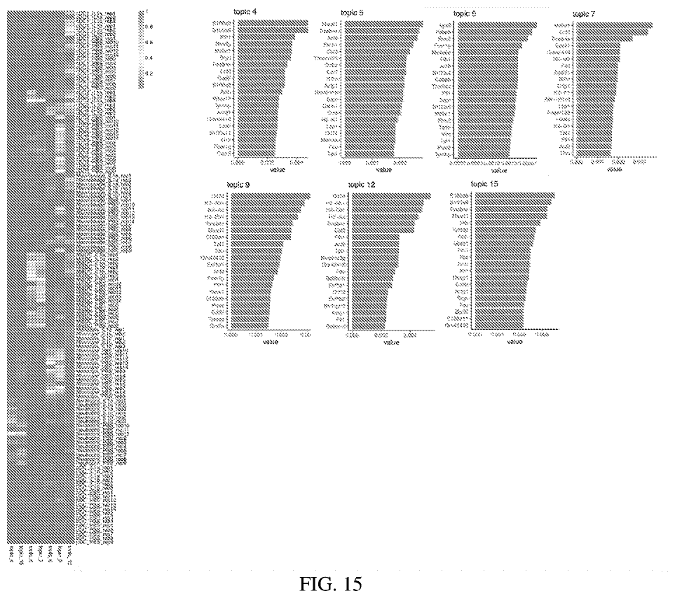

The results show that the distinct responses to cytokines across cell types are universal. An example shows that IL1b stimulation results in distinct gene expression signatures in macrophages and DCs (FIG. 14). To further assess the commonality and differences in response to IL1a and IL1b, topic models were used to study gene programs that are up- or down-regulated post cytokine stimulation. Of the 20 topics identified, it was found that 5 of them change in expression post-stimulation, suggesting that these gene programs are related to cell activities rather than identities or technical factors (FIG. 15). While monocytes and cDC2 share a topic to an extent, all other cytokines have unique topics. The neutrophil topics most strongly associate with inflammatory and chemokine genes, consistent with their role as first responders to inflammation triggers. cDC1 cells upregulate programs related to antigen presentation. Migratory DCs upregulate cell type identity programs including Ccr7, suggesting that their cell type functions are reinforced during acute inflammation induced by IL1. The receptors for IL1 are most highly expressed by neutrophils and are also upregulated post IL1 stimulation.

### Identification of Cellular Polarization States by Cytokine Stimulation

Because the compendium represents a large number of immune stimuli, possible cellular polarization states in an acute immune response were identified. Certain cell types, such as macrophages, have been characterized in detail regarding their polarization state in response to a small number of cytokines. However, the polarization states of most other cell types responding to different stimuli are yet to be characterized. NK cells, which are known for having multi-functionality, were examined first. How NK cells respond to many different immune stimuli has not yet been characterized. The gene expression level of genes associated with cytotoxicity (Prf1, Gzma, Gzmb, Gzmc, Lamp1, etc.) was examined, and it was found that many cytokines induced a strong upregulation of these genes, with the strongest drivers being IL-2, 3, 7, 12, 15, 27, 33, 36a, interferons and GM-CSF. As NK cells are known to play an important role in anti-tumor and anti-viral immunity, these cytokines could serve as potential therapies to be used in combination with existing therapies to improve an NK cytotoxicity response.

Next, an unbiased approach was taken to cluster all NK cells from all stimulations and PBS conditions to identify possible polarization states. A graph-based clustering approach was used, and 7 clusters of NK cells were identified. Several of these clusters are highly enriched in cytokine stimulated conditions compared to the PBS control. For example, it was found that cluster 3 and 4 correspond to the elevated cytotoxic response as previously described. However, one cluster containing predominantly IL-18 stimulated cells, had a markedly different response, with upregulated genes representing growth and proliferation. Another cluster contains predominantly IL-1a and IL-1b stimulated cells and is marked by elevated IFNgr expression. Furthermore, IFNs polarize cells into an anti-viral state in another cluster. In conclusion, NK cell polarization states with different activities were classified, and drivers for each polarization were identified. Similar analyses were performed for other cell types, and their polarization states were also characterized.

### Inferring Receivers by Cytokine Signatures

Based on the observation of cell type specific signatures to cytokines, an in silico method was created to infer cytokine impact. A scoring system that ranks cytokine impact by comparing it with the cytokine signature was created, and a deconvolution method that sums a gene expression profile to be deconvolved into cytokine signatures that sum up to 100% was also created. The cytokine signature enrichment method was evaluated by testing them on the combinatorial cytokine injection (IL2/IL15; IL2/IFNG) as well as cytokine profiles themselves, and a high specificity for cytokines was inferred. After validation, the inference algorithm was used to study the impact of cytokines onto each cell type post vaccination. Inference results show that interferon is a major component of early vaccine response, which is corroborated based on observations that interferon stimulated genes are highly upregulated post vaccination.

Three types of methods for Cytokine Response Enrichment Analysis (CREA) were developed to assess cytokine response in a user's data depending on the input, allowing for a great level of flexibility of user inputs: 1) CREA-ORA: user only inputs a list of gene symbols of interest, and the method performs signature enrichment using hypergeometric test-based overrepresentation analysis (ORA) commonly used for gene ontology (GO) pathway enrichment; 2) CREA-dot: user inputs gene symbols of the entire transcriptome along with the weights of each gene, and enrichment is computed using a normalized dot product score; 3) CREA-decon: user inputs single cell gene expression matrices, and the enrichment is holistically assessed for all cytokines by deconvoluting user data into component cytokine signatures.

Cytokine Production is Cell Type Specific and Inversely Correlated with Cell Type Abundance

Next, the production of cytokines was examined based on single cell RNAseq data of cells collected 4-6 hours post in vivo stimulation with 92 different types of stimuli, including the 86 cytokines described above as well as 5 agonists for pattern recognition receptors and 1 peptide, representing most of the common immune stimulators. RNA detection sensitivity was normalized by normalizing the RNA transcripts mapped to cytokine genes across all cell types. Cytokine production across each cell type was mapped, and it was found that the majority of cytokines (90%) are produced by less than 4 distinct cell types. It was found that the overwhelming majority of cell types that produce a specific cytokine after stimulation already express the transcripts in homeostasis. The production of cytokines per cell type was mapped. The cell type specificity of cytokine production enables inference of cytokine producers in the majority of physiological systems.

Rarer cell types in lymph nodes, such as basophils and neutrophils, produce a large number of cytokines. Thus, it was investigated whether there is any correlation between the number of cytokines expressed by a cell type and the abundance of the cell type in lymph nodes. A strikingly strong and significant inverse relationship between the number of cytokines a cell type produces and the abundance of the cell type was found (Pearson rho=−0.78, p=5e-5). Moreover, the rare cell types that account for only less than 5% of lymph node populations are responsible for more than 75% of distinct cytokine production. This suggests that rarer cell types are critical players in the immune cell-cell communication network and may suggest an evolutionary mechanism of the mammalian immune system to regulate cytokine production and to restrict cytokine storms.

### Inference of Cell-Cell Communication Networks

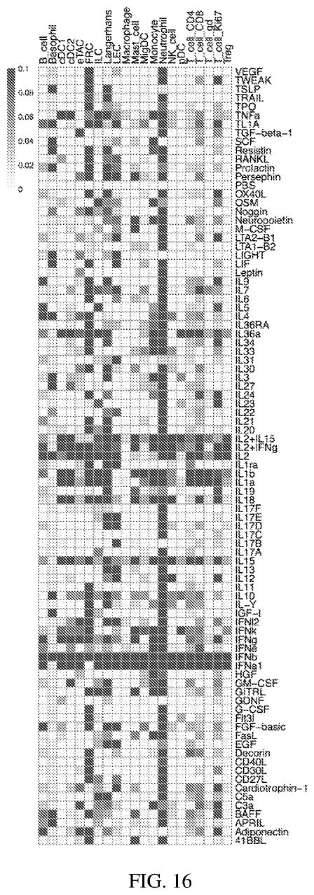

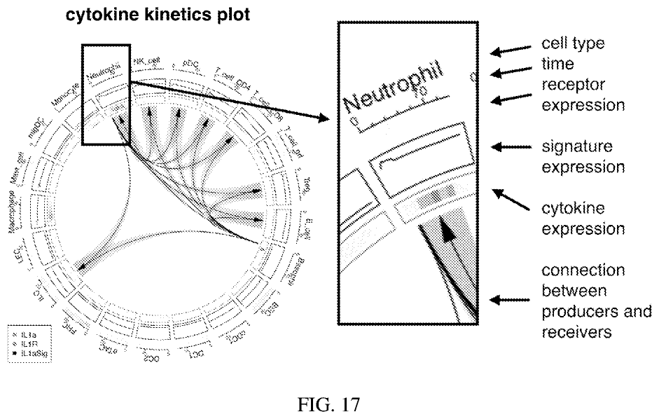

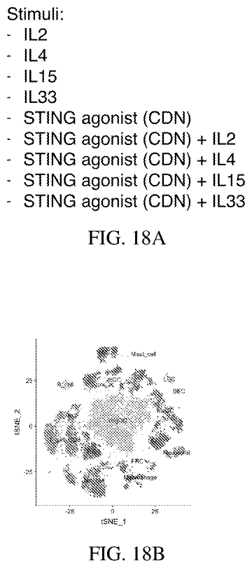

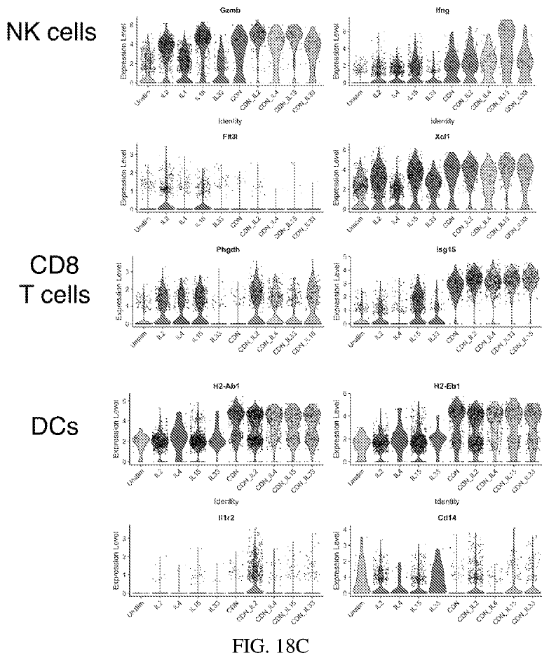

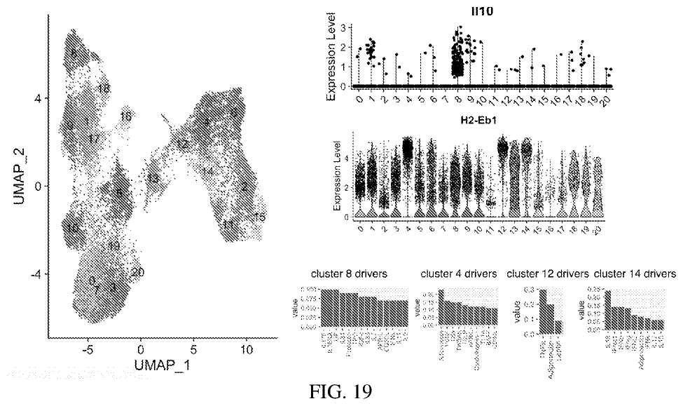

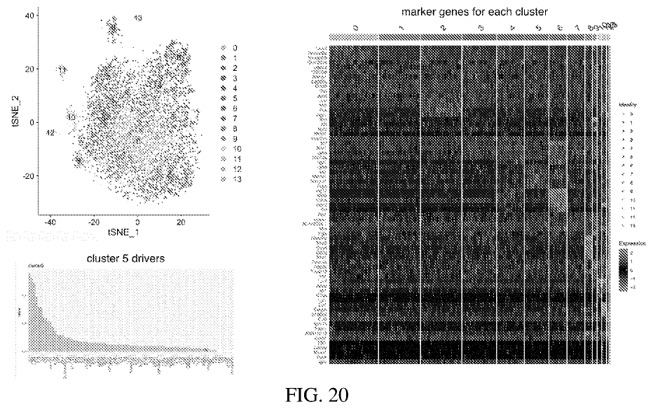

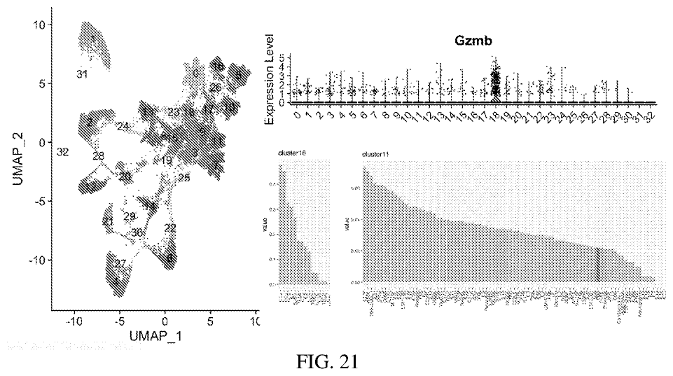

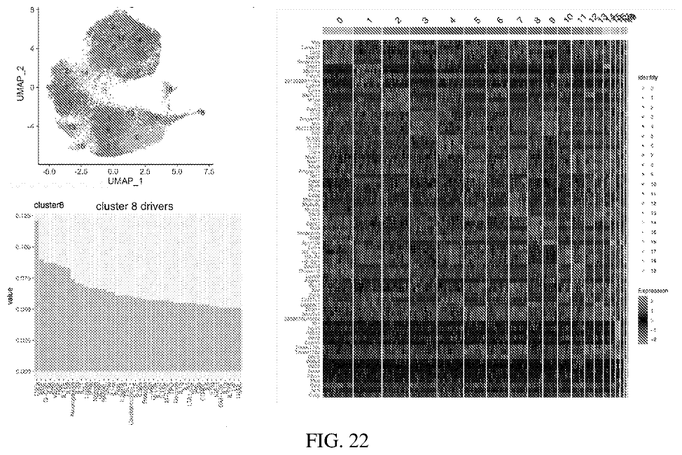

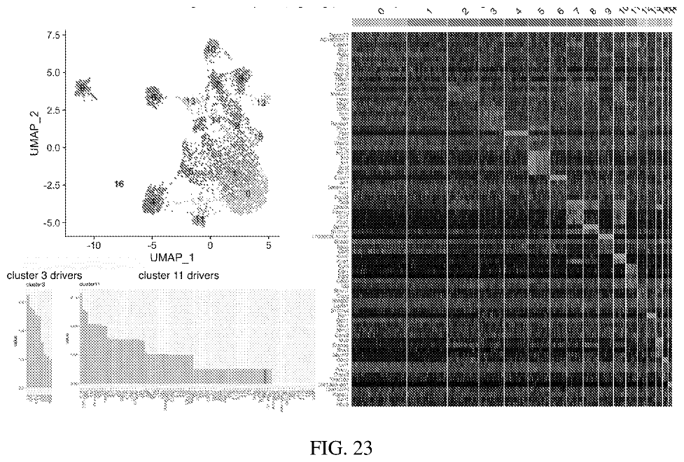

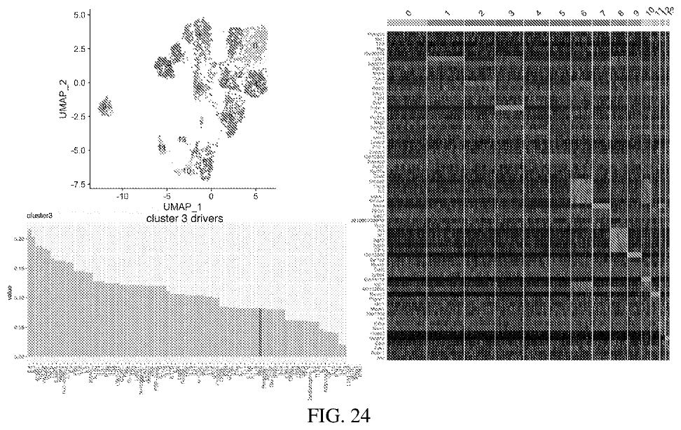

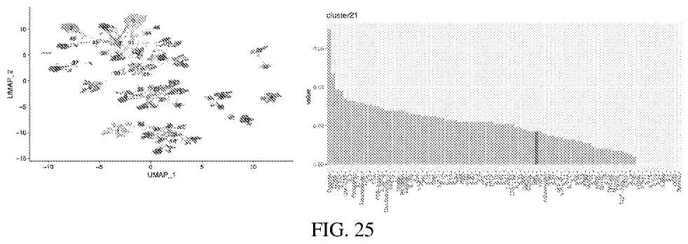

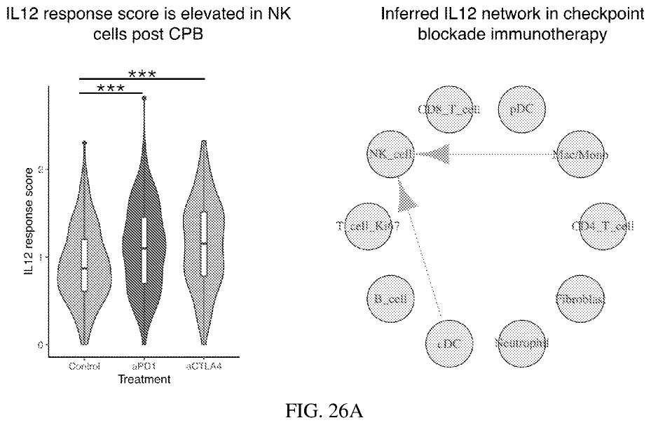

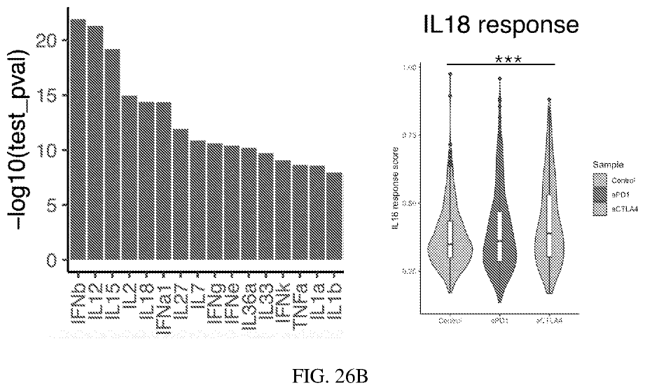

Following mapping of both cytokine receivers and cytokine producers, a method for studying cell-cell communications in a physiological system was created. Visualization methods for static and dynamic cell-cell communication systems were created and named cytokine communication plot and cytokine kinetics plot, respectively. The ligand expression, receptor expression, impact score and inferred cell-cell communications were visualized. This database was used to infer cell-cell communication in another study, where vaccines consisting of OVA peptides and STING agonist as an adjuvant were administered, and 12 time points were profiled by single cell RNA sequencing post vaccination. It was found that IFNa is produced by macrophages at an early time point, and the signal is transmitted to all other cell types, leading to a collective anti-viral response across all cell types. It was also found that IL1a is produced by neutrophils and macrophages, but mostly targets T cells and neutrophils, with very little response in monocytes (FIG. 17). This enabled identification of key drivers in an immune response by reconstructing the cell-cell communication networks of a complex immune response using the individual stimulation signatures.

In summary, a large compendium of cytokine signatures was created, providing a reference for functional studies of cytokines. The use of this reference was demonstrated for identifying novel cytokine functions. Additionally, an unbiased bioinformatics approach was used to systematically compare cell-type-specific responses to many cytokines, and to uncover polarization states of each cell type and the cytokines capable of inducing each polarization state. Finally, the database and bioinformatics algorithms created can be readily applied to other datasets, especially single cell transcriptomic profiling datasets, allowing researchers to uncover cytokine responses and cell-cell communication networks in any immune response. Future studies can examine the dose-dependent cytokine responses by varying dosage and length of stimulation.

### Methods

Cytokine injection: First, 86 cytokines were selected, representing most of the cytokines within each major family (i.e., common γ chain (IL2/4/7/9/13/15/21), IL6 (8 members), β common (GM-CSF/IL3/IL5), IL10 (5 members), TNF (17 members), IL17 (6 members), IL12 (3 members), complement (C3a, C5a), IL1 (7 members), and growth factors (10 members); these represent a subset of the 86 cytokines).

To preserve cytokine activities, carrier-free cytokines were freshly reconstituted according to the manufacturer's recommendations, stored under 4° C., and used within 28 hours post-reconstitution. 5 μg of each cytokine was injected in 100 μl of PBS at known bioactive concentrations based on prior literature on individual cytokines. Cytokines were injected under the skin (50% subcutaneous, 50% intradermal) bilaterally in the abdominal flank of distinct mice (with 3 replicate C57BL/6 mice per cytokine to ensure reproducibility). The 3 replicates of each cytokine were performed in different batches. The draining lymph nodes were then harvested 4 hours post injection; the time point was chosen based on prior studies to ensure that there is enough time for the majority of genes to become differentially expressed post-stimulation, yet to be early enough to minimize secondary effects that are not specific to the ligand. All experiments were harvested at 6-8 am to exclude the impact of circadian clocks on transcriptomic profiles. All experiments have been reviewed and approved by the Broad Institute Animal Care and Use Committee (IACUC).

Single cell gene expression experiment: The lymph node cells were processed using an optimized protocol for viable cell recovery and sample multiplexing, and single cell emulsions were generated using the 10× Genomics droplet-based Chromium instrument and sequenced on an Illumina NovaSeq S4. Cytokine samples were randomized and processed in the same sequencing runs to avoid systematic batch effects. PBS controls are included in every batch.

Single cell data pre-processing: The sequencing reads were aligned to the mm10 mouse genome reference and the transcriptomic count matrix was assembled using the CellRanger software. The Seurat R pipeline was used to perform quality control, and 399,861 cells that passed quality control were identified (defined as cells with >200 and <8000 genes, >500 UMIs and <10% mitochondrial content). Standard scRNA-seq data processing was then performed, including normalization, variable gene selection, principal component analysis, and tSNE visualization on the high-quality cells.

Cell clustering: SNN-cliq, a graph-based clustering method, was used to partition cells into clusters, and a cell identity was assigned to each cluster based on genes uniquely expressed by the cluster. Sub-clustering for each major lineage (e.g., B cells, T cells, myeloid cells) was optimized to have enough cells per cluster to find reproducible responses per cytokine. At least 17 cell populations were identified, including CD4 T, CD8 T, Tregs, gdT, NK cells, cDC1, cDC2, migratory DC, pDC, lymphatic and blood endothelial cells, fibroblastic reticular cells, macrophage, monocytes, neutrophils, mast cells, ILCs. For each cytokine in each cell type, preliminary gene expression difference vectors in each animal were assembled, and Pearson correlation coefficients between the three animal replicates were computed, demonstrating highly similar stimulation-induced changes in gene expression across biological replicates.

Defining the transcriptomic response of each cell type to each cytokine: For each defined cell type, genes will be identified that are differentially expressed between cytokine-stimulated samples and PBS control samples (Wilcoxon rank sum test). Both single-cell replicates and animal replicates are addressed in the analysis. For single cell replicates, the same number of cells will be sampled to ensure comparability of statistical tests across cytokines. Significantly differentially expressed genes are defined by the following criteria: genes that are greater than 0.5 fold change with FDR less than 0.25 at the single-cell replicates level. The average fold change for each gene will be considered the weight of the gene in this signature, with a higher absolute weight representing more significant changes in gene expression under this cytokine stimulation. The output from this subaim are vectors of fold changes for significantly regulated genes, with each vector representing a transcriptomic response per cytokine per cell type (i.e. 86 cytokines×17 cell types=1462 response vectors).

Quantification of cell type specific transcriptomic responses to each cytokine: For each cytokine and each cell type, differential gene expression between cytokine-stimulated and PBS control of the cell type was computed. The heatmap of divergence of transcriptomic responses to each cytokine across cell types is shown. Divergence is calculated based on symmetric KL divergence (or other distance metrics include the Euclidean distance, cosine distance) between cytokine response vectors and PBS vectors in a low-dimensional space.

Cytokine impact score: A weighted scoring approach was created to rank the impact of cytokines in a system by comparing the cell-type-specific cytokine signature generated in this study and in a system of interest.

Cytokine production score: Cell type specific productions of cytokines were assessed using the 86 cytokine stimulations obtained from this study as well as 6 other stimulations, resulting in 92 stimulations representing most of the common immune stimulations within 4-6 hours. Normalized mRNA counts for each mRNA were mapped to a cytokine, and the relative expression of cytokines across cell types was assessed.

Cell-cell communication network construction: Models of cell-cell communication networks were constructed by taking into account cytokine production and cytokine response. Cytokine production is obtained by examining the transcripts mapped to the cytokine of interest in a system, and cytokine response is obtained by calculating the cytokine impact score by comparing gene expression signature in a system of interest and the cell-type-specific cytokine signature created in the study.

Computational method 1: A computational method (referred to as “CREA-decon”) was developed to deconvolute a target profile using a linear combination of individual profile. The coefficients of the combination indicate the relative importance of their respective cytokines. Y=Xβ, where Y=normalized target profile, X=normalized individual profiles, and β=coefficient to be estimated. To estimate β, a regularized regression was used with L1 and/or L2 penalties, corresponding to the lasso and/or ridge regression methods. β=argmin_{β} ∥Y−Xβ∥²+regularization terms on β, e.g., β=argmin_{β} (∥Y−Xβ∥²+λ₂∥β∥²+λ₁∥β∥₁). The resulting beta vector would be the length of the number of cytokines in the stimulation data, and the higher number a particular β value is, the more important the corresponding cytokine is. This general method can be modified in the following ways: 1) include only genes associated with response to a particular stimuli (instead of the entire transcriptome) to increase signal and reduce noise; 2) find such genes by computing AUROC when comparing a cytokine stimulated profile vs. all other profiles and only include the genes with AUROC score above a cutoff; 3) for x and y, instead of a single vector describing the mean or median (or any form of average) of profiles, can have a distribution over all cells to describe the distribution.

Computational method 2: A computational method (referred to as “CREA-dot”) was developed to give a cytokine enrichment score for each cytokine, rank all cytokines based on their enrichment score, and compare the enrichment score with a random background distribution to assess the statistical significance of enrichment. The enrichment score is computed by taking the inner product between the normalized target profile and normalized individual profiles. Y is defined as the normalized target profile and X as the normalized individual profiles (e.g., scores=X^{T} Y). Background random distribution is obtained by randomly shuffling gene labels in the target profile and normalized individual profiles many times. A score is considered significant if it is higher than 95% of the random scores after accounting for statistical multiple testing adjustment. This general method can be modified as described for computational method 1 above.

Computational method 3: A third computational method (referred to as “CREA-ORA”) was developed. Genes that are significantly up- or down-regulated in an immune response (for example, pre- vs. post-treatment) are computed. The number of genes that overlap with each of the cytokine-induced signatures was found. A hypergeometric test was used to identify the statistical significance of the overlap. The more overlap there is, the more likely that cytokine is driving the observed response (e.g., driving a treatment response). Those cytokines can be used to combine with existing therapies that lead to an enhanced response.

### Example 2

### A Dictionary of Immune Responses to 86 Cytokines In Vivo at Single-Cell Resolution

Cytokines mediate cell-cell communication in the immune system and represent important therapeutic targets.¹⁻³ While there have been in-depth studies of individual cytokines, a global view of the responses of each major immune cell type to each cytokine is lacking. To address this gap, the Immune Dictionary—a compendium of single-cell transcriptomic profiles of over 20 cell types in response to each of 86 cytokines in murine lymph nodes in vivo—was created. A cytokine-centric view of the dictionary revealed that most cytokines induce highly cell-type-specific responses. For example, the inflammatory cytokine IL-10 induced distinct changes in gene expression in each of >10 cell types. A cell type-centric view identified both known and previously uncharacterized cytokine-induced polarization states in every cell type, such as a distinctive polyfunctional state of NK cells induced by IL-18. Based on the dictionary, a companion software, Immune Response Enrichment Analysis (IREA), was developed for assessing cytokine activities in any transcriptomic data. It was applied to infer cytokine production and response in each immune cell type based on single-cell transcriptomes measured in tumors following checkpoint blockade therapy. The dictionary generates new hypotheses for cytokine functions, illuminates the pleiotropic effects of cytokines, expands knowledge of activation states in each immune cell type, and provides a framework to deduce the roles of specific cytokines and cell-cell communication networks in any immune response.

Cytokines are secreted by cells and bind to cognate receptors locally or at a distance, triggering downstream signaling pathways and orchestrating coordinated actions among different cell types of the immune system. Many individual cytokines have been studied in depth⁴⁻⁹, and several studies of limited scope on cellular responses to cytokines have been performed^{10,11,12}. However, a large-scale systems-level framework for comparing cellular responses to each cytokine in each immune cell type has not been developed. With advances in single-cell transcriptomics (e.g., scRNA-seq) technologies, hundreds of thousands of cells can now be profiled in a microenvironment simultaneously to discover cell subsets, states, and programs^{13,14}. Such technologies can enable a robust systems-level framework for explaining how coordinated immune responses arise from cell-cell interactions mediated by secreted factors.

### A Single-Cell Dictionary of Cytokine Responses

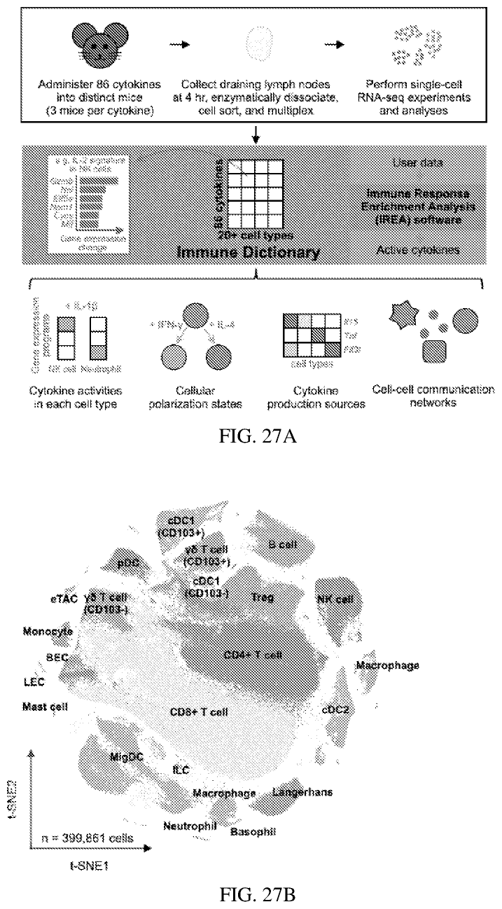

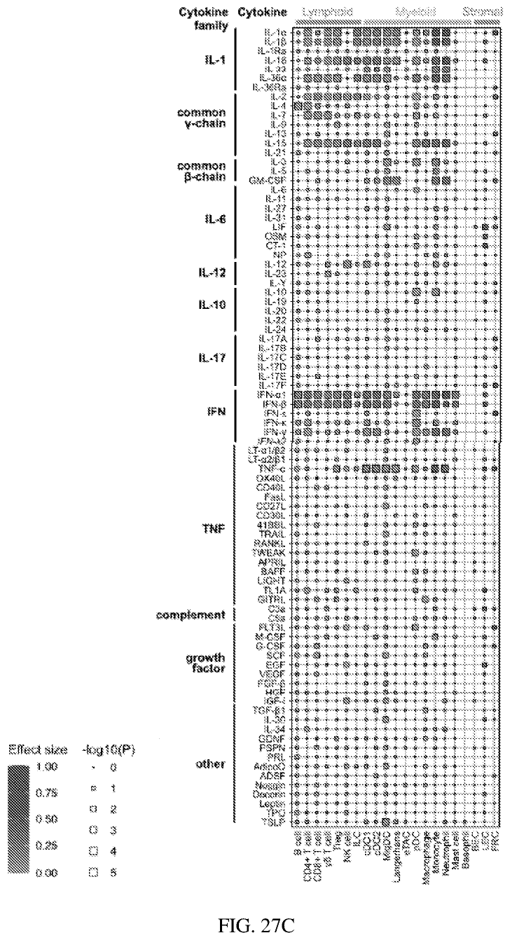

To obtain a comprehensive view of cellular responses to each cytokine, a dictionary of immune signatures was created. Single-cell transcriptomic responses were systematically profiled in vivo to 86 cytokines across over 20 cell types in mouse lymph nodes, representing >1500 cytokine-cell type combinations (FIG. 27A). The term cytokine is used to broadly refer to secreted molecules that coordinate signaling of the immune system and include a small number of growth factors and hormones that also have cytokine functions. The 86 cytokines representing most of the members in each major cytokine family were selected, including IL-1 (IL-1α/1β/Ra/18/33/36a/36Ra), common γ chain (IL-2/4/7/9/15/21), common β chain (GM-CSF/IL-3/IL-5), IL-6 (IL-6/11/27*/31, LIF, OSM, CT-1, NP), IL-12 (IL-12/23/27*/Y), IL-10 (IL-10/19/20/22/24), IL-17 (IL-17A-F), interferon (IFN-α1/β/ε/κ/γ/λ2), TNF (TNFSF1-15, 13B, 18), complement (C3a, C5a), and a small number of representative cytokines from other protein families with less well-characterized immune functions (e.g., Persephins, Adiponectin).

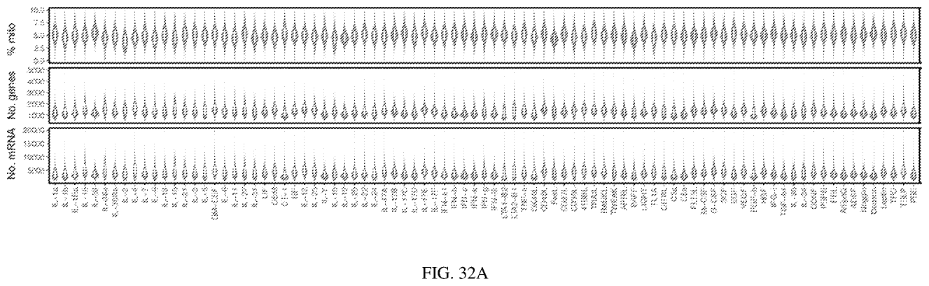

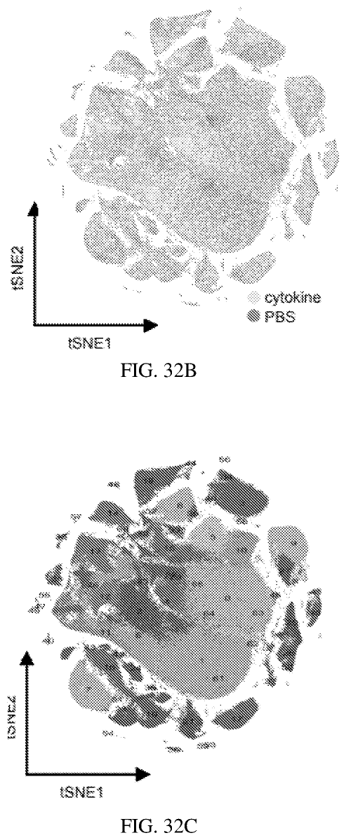

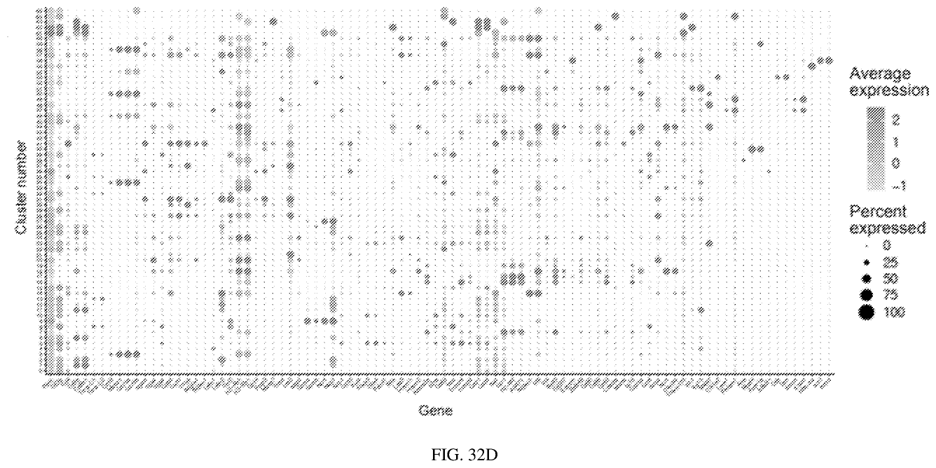

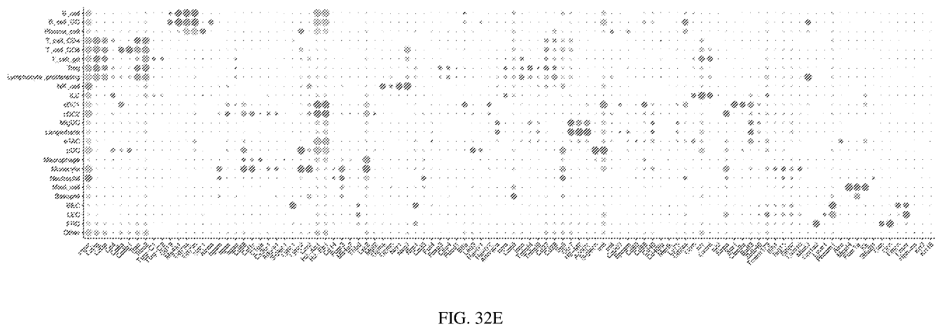

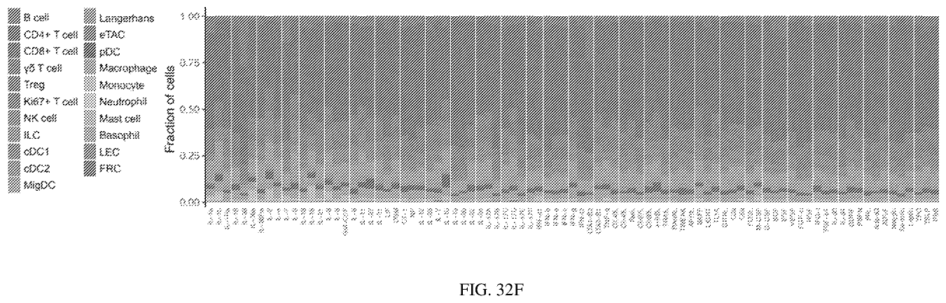

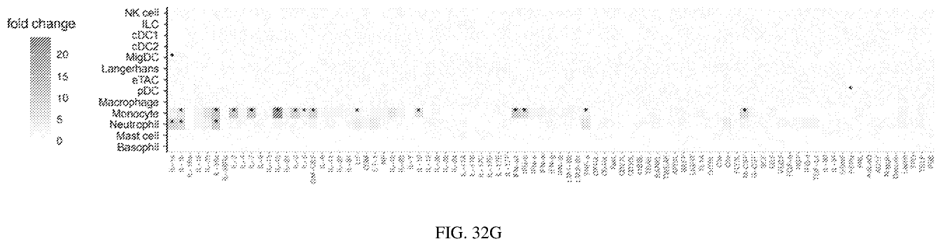

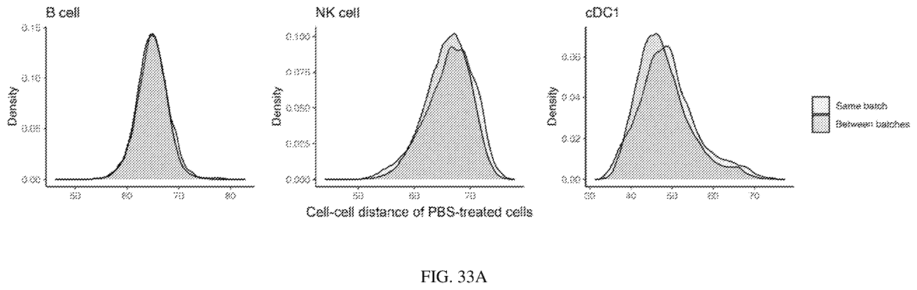

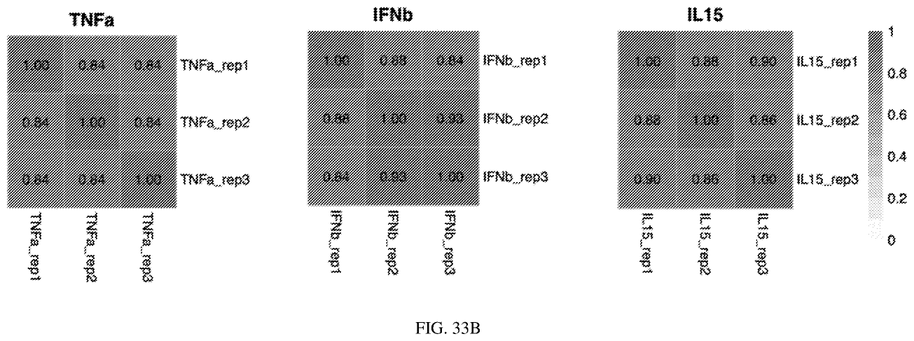

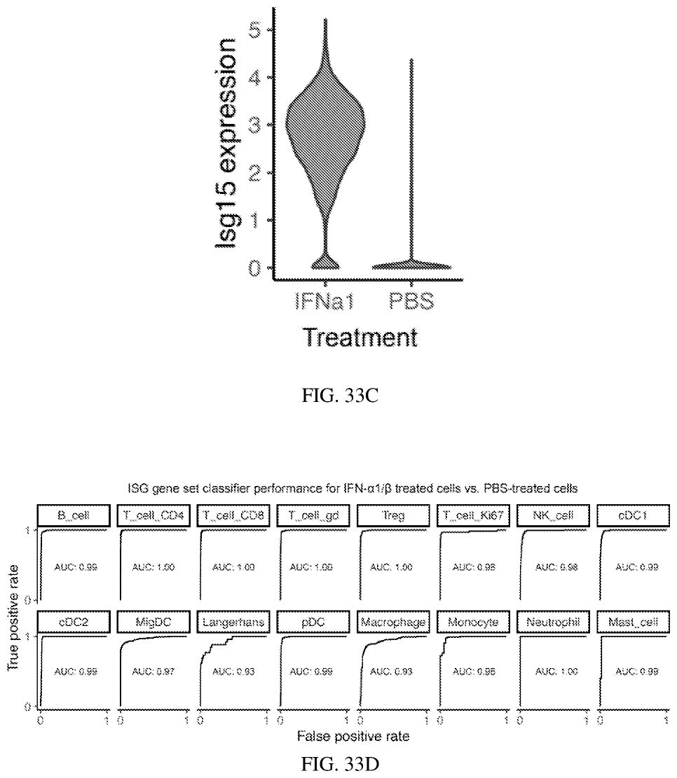

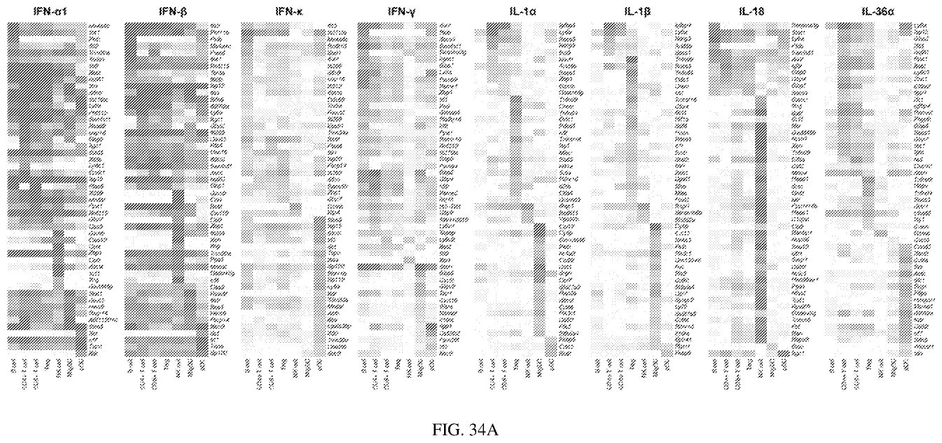

Each freshly reconstituted carrier-free cytokine was injected under the skin in the abdominal flank of distinct mice (with 3 replicated wild type C57BL/6 mice per cytokine). Based on prior literature on individual cytokines, a high dose of the cytokine was administered to maximize the chance of inducing an observable gene expression change. To ensure adequate time for the majority of the transcriptome to respond post-stimulation, yet to be early enough to minimize secondary effects that are not specific to the cytokine, the draining lymph nodes, where immune cells converge to initiate immune responses, were harvested four hours post-injection. The lymph nodes were then processed using an optimized protocol for viable cell recovery, balanced cell type representation, and high-throughput sample multiplexing. Data quality, including batch-to-batch consistency, was experimentally strictly controlled for and computationally verified. Single-cell profiling of harvested lymph node cells was performed using a droplet-based system (10× Genomics) to generate single-cell transcriptomes for 399,861 cells that passed quality control (FIG. 27B, FIG. 32, FIG. 33).

After partitioning cells into clusters, most cells were found to cluster by cell type identity rather than stimulation conditions (FIG. 32B). While cytokine-treated cells did not typically form distinct clusters, they were often separated from PBS controls within each cell type cluster. Upon sub-clustering of each major cluster and manual inspection to ensure the accuracy of cell type identification, more than 20 cell types were found, corresponding to B cell, plasma cell, CD4+ T cell, CD8+ T cell, γδ T cell (Cd103+ and Cd103−), regulatory T cell (Treg), NK cell, ILC, cDC1 (Cd103+ and Cd103−), cDC2, migratory DC (MigDC), Langerhans cell¹⁵, extrathymic Aire-expressing cell (eTAC)¹⁶, pDC, macrophage, monocyte, neutrophil, mast cell, basophil, blood endothelial cell (BEC), lymphatic endothelial cell (LEC), and fibroblastic reticular cell (FRC) (FIG. 27B, FIG. 32, FIG. 33). The resulting transcriptomic responses to 86 cytokines across over 20 lymph node cell populations constituted a dictionary of immune responses (‘Immune Dictionary’).

To map the responses to each cytokine per cell type, changes in transcriptomes between cells from cytokine-treated relative to PBS-treated mice were quantified (FIG. 27C), and found the changes to be consistent across biological replicates (FIG. 33B). A subset of cytokines (IL-1α, IL-1β, IL-15, IL-18, IL-36a, and TNF-α) were found to regulate gene expression across many cell types, with IFN-α1/β affecting almost every cell type and a small number affecting myeloid or lymphoid cells more selectively. The remaining cytokines influenced a small number of cell types. These results provide a global reference map of cell types responding to each cytokine in vivo.

### Cell-Type-Specific Cytokine Responses

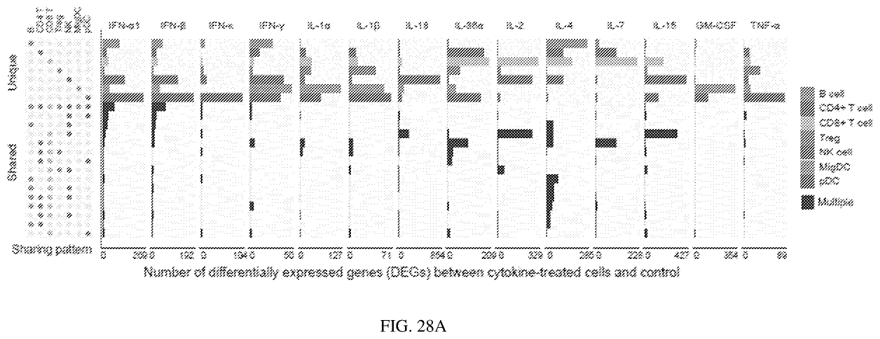

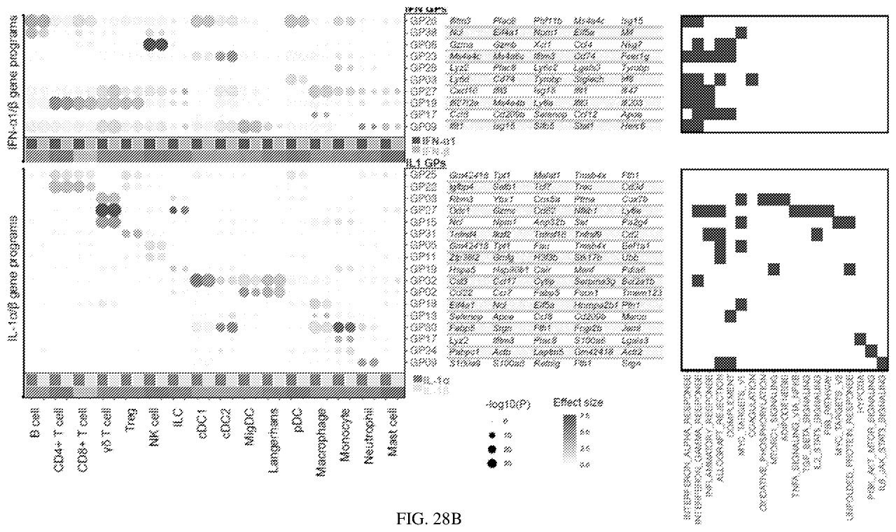

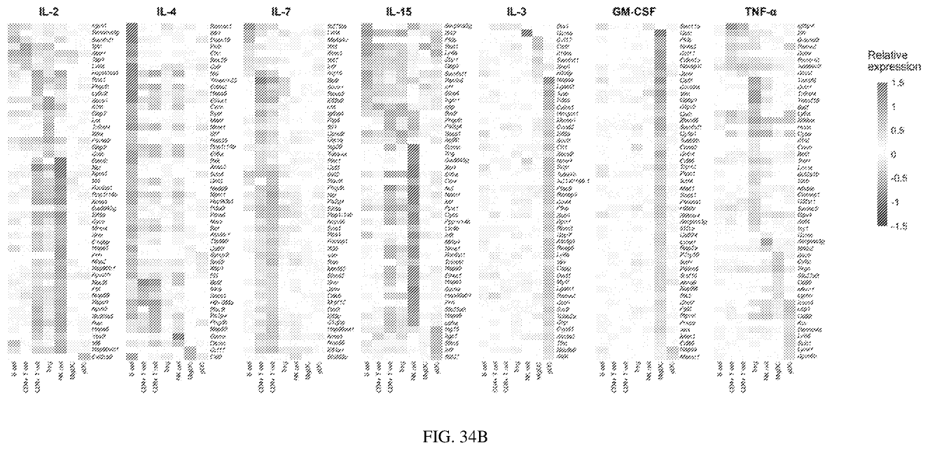

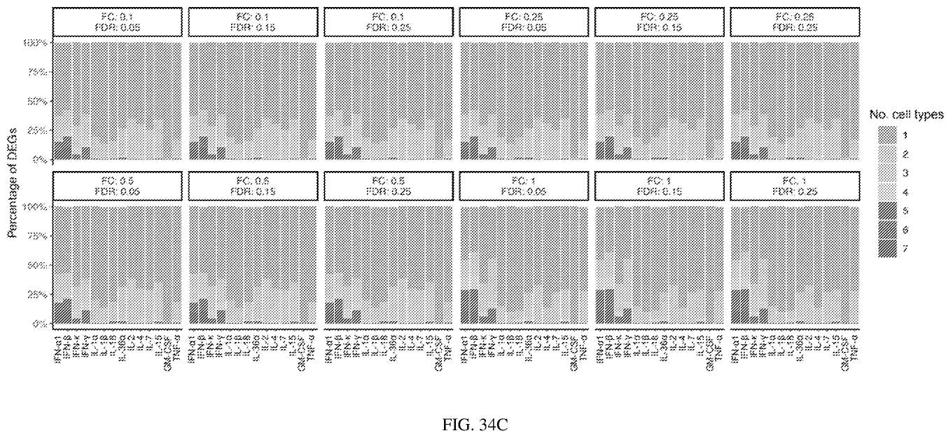

A cytokine-centric view of this dictionary was taken to explore how different cell types respond to the same cytokine. Seven cell types that were sufficiently and similarly abundant to enable comparative analysis of cytokine responses were investigated, and 14 cytokines that induced strong transcriptomic changes in a large number of cell types were also investigated. The number of differentially expressed genes (DEGs) that were either unique to each cell type or shared between multiple cell types was computed (FIG. 28A, FIG. 34A). The vast majority of the DEGs to a particular cytokine were found to be highly specific to one cell type, regardless of thresholds for defining DEGs (FIG. 34B). The most shared DEGs were found for interferons (which induced ISGs across all 7 cell types), IL-4 (which induced shared genes across several combinations of cell types), and IL-2, IL-15, and IL-18 (which induced cytotoxic genes in CD8+ T cells and NK cells).

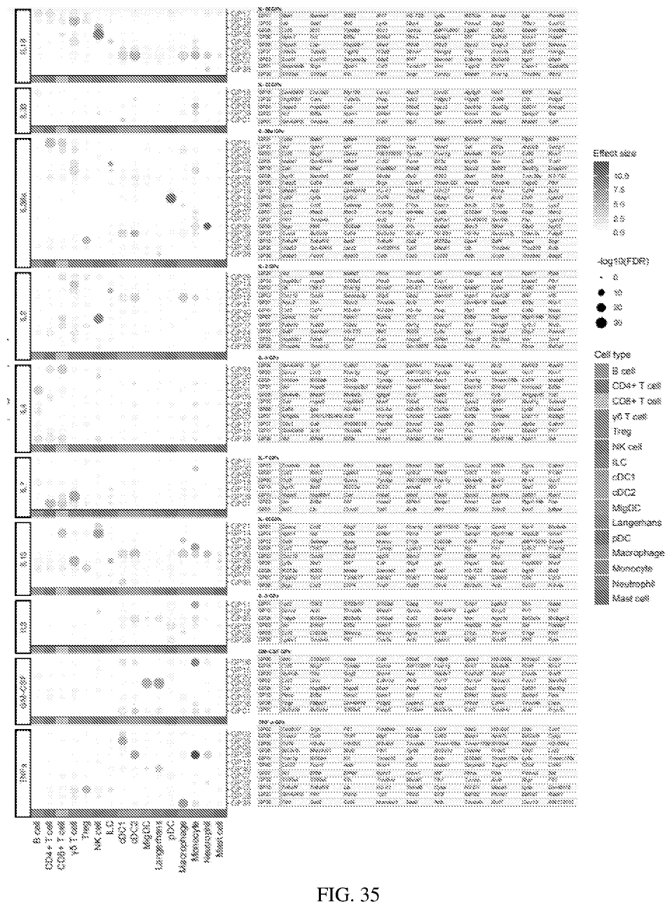

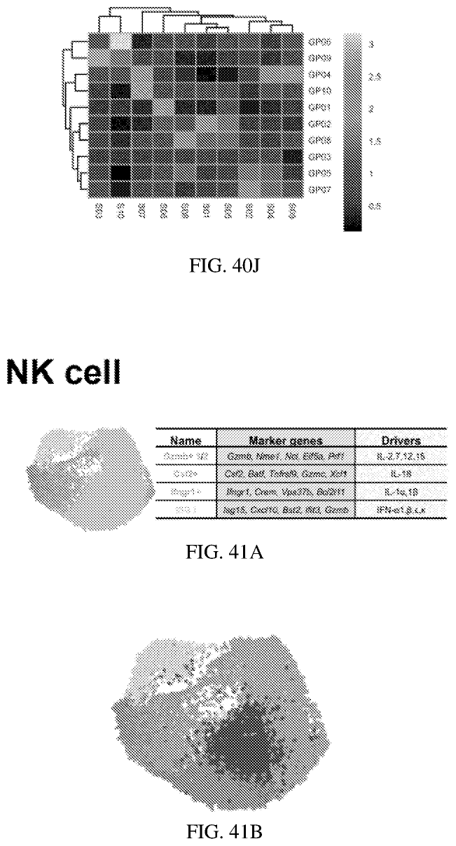

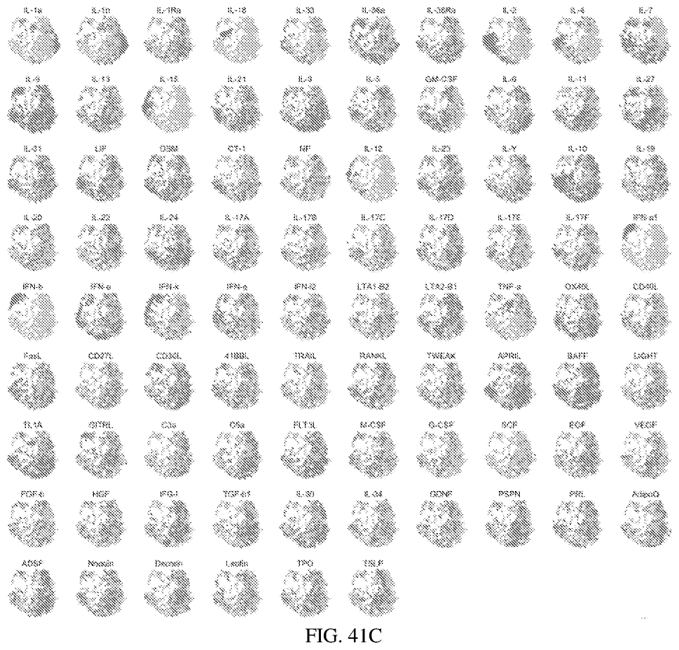

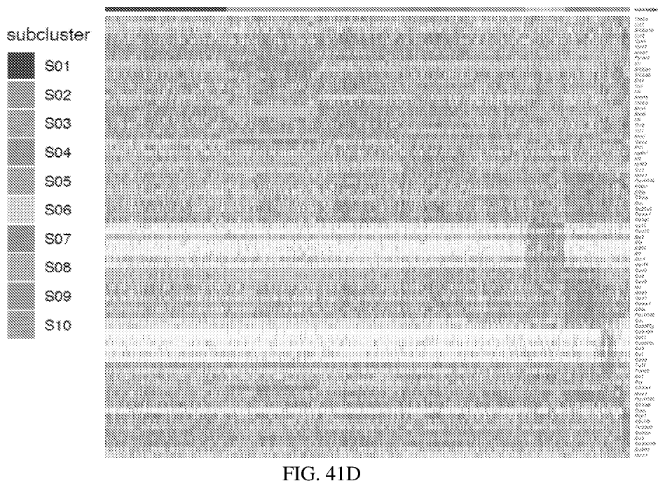

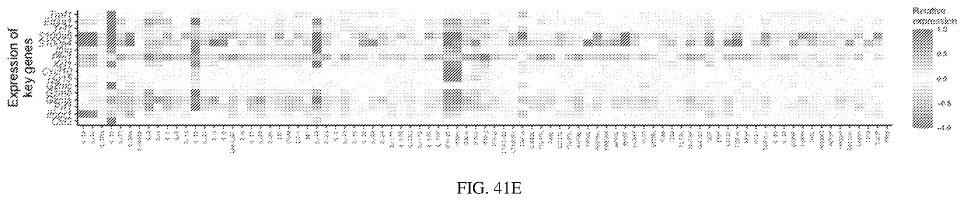

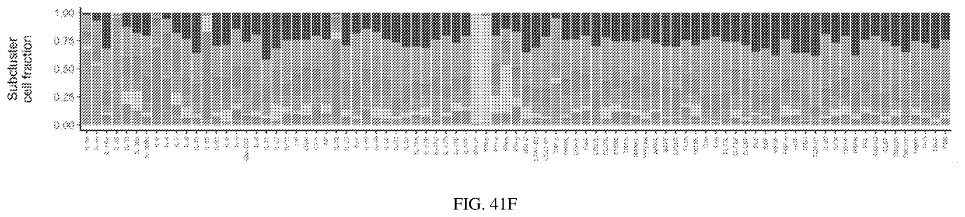

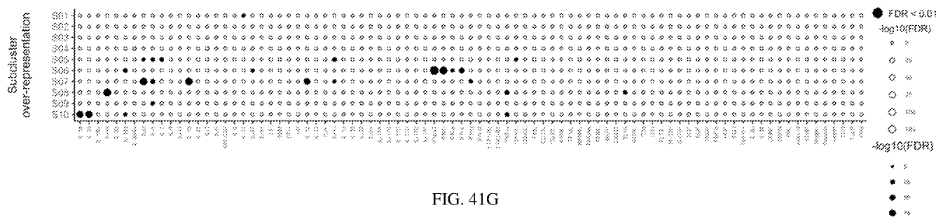

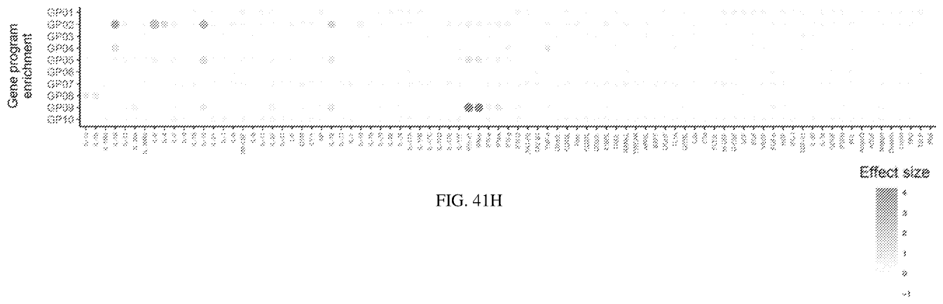

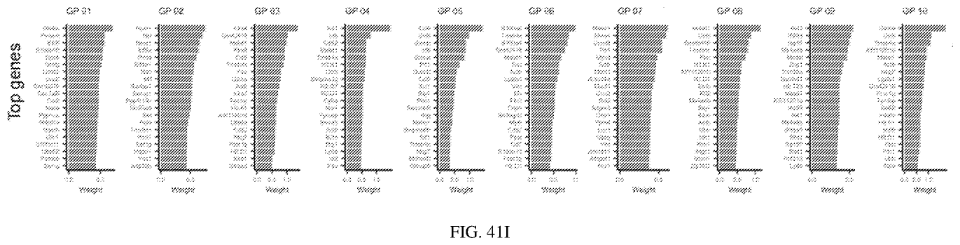

Notably, it was found that IL-18 induced an upregulation of 1,138 genes in NK cells, which is an order of magnitude more genes compared to IL-18 responses in other cell types (FIG. 28A, FIG. 35). Some of these genes are also induced by other cytokines, such as cytotoxic molecules Gzmb and Prf1, while others are only induced by IL-18, such as GM-CSF/Csf2 that can promote myeloid cell proliferation and maturation (FIG. 41E). IL-18 has recently shown promise in preclinical studies of cancer immunotherapy¹⁷, and is known to activate T cells, NK cells and other cell types¹⁸. Observation of a strong NK cell response to IL-18 suggests an important role for the IL-18-NK cell axis in a polyfunctional immune response.

Gene programs (GPs) were then identified across all cell types, which consist of co-expressed genes found using non-negative matrix factorization (NMF)¹⁹, and GPs that are induced by cytokines were investigated further (FIG. 28B-28C, FIG. 35). IFN-α1 and IFN-β, as expected, induced common GPs across many cell types but with some lymphocyte- and myeloid-specific programs. IL-1α and IL-1β, which are potent proinflammatory cytokines with many known functions in the activation of both innate and adaptive immune cells²⁰⁻²³, were found to induce similar responses to each other across all cell types. However, in contrast to IFN-α1/β that induced highly overlapping ISG programs across all cell types, IL-1α/β triggered upregulation of highly cell-type specific GPs with a diverse set of enriched biological processes. These GPs appeared to enhance the known functions of these cell types, rather than induce common processes: (i) neutrophils upregulated inflammatory and chemokine genes as part of GP09 consistent with their role as first responders; (ii) cDC1 up-regulated GPs related to antigen presentation; (iii) migratory DCs up-regulated cell type identity programs including Ccr7; and (iv) Tregs upregulated Hif1a and Tnfrsf18, which encodes glucocorticoid-induced TNFR-related protein (GITR), potentially mediating immune tolerance during T cell activation. TNF, and some other members of the IL-1 cytokine family, including IL-36a, also displayed such highly cell-type-specific transcriptional responses (FIG. 34A, FIG. 35C, FIG. 35J). The contrast between type I interferon and IL-1 responses illustrates how interferons induce a common and autonomous viral defense program while IL-1 triggers a coordinated multicellular response that reinforces cell-type-specific functions. Systematic analysis of how different cell types respond to cytokines provide a molecular map for the observed pleiotropic effects of cytokines²⁴.

### Cytokine-Induced Cell Polarization States

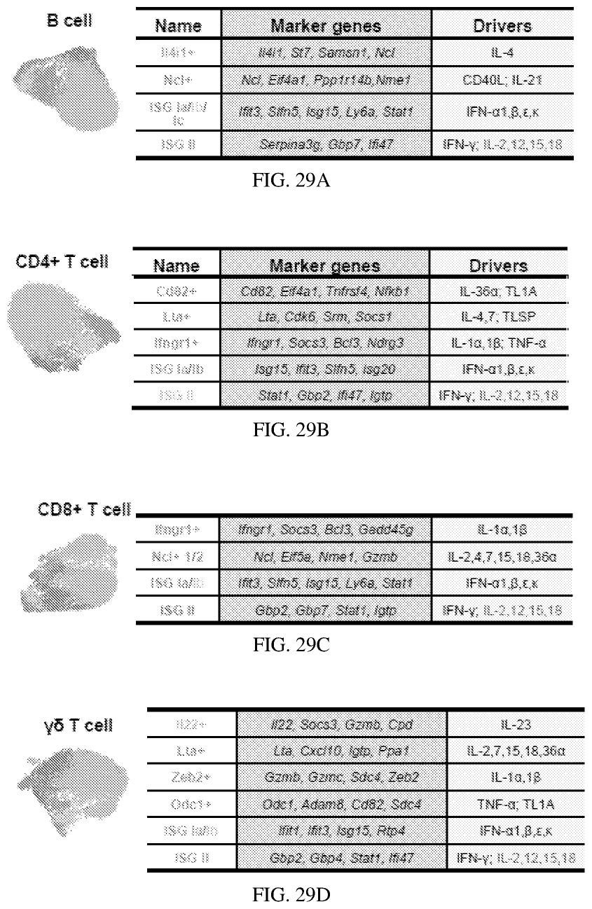

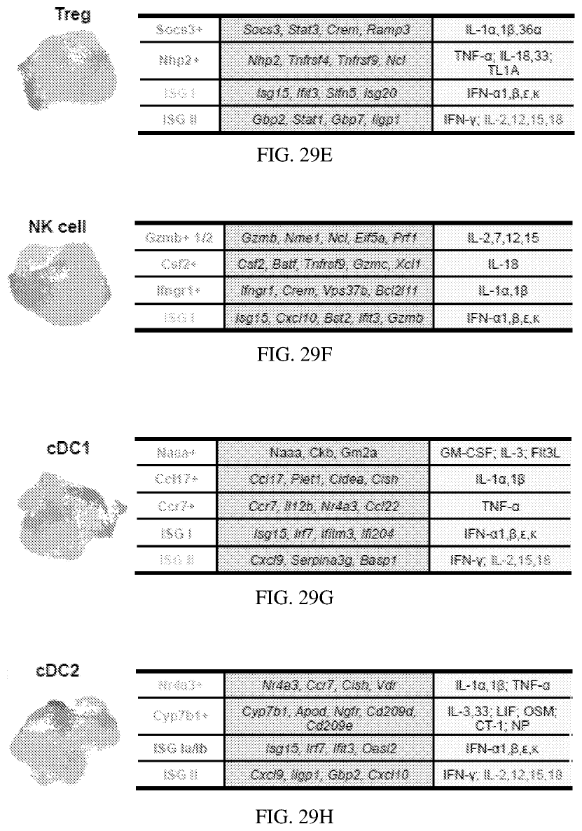

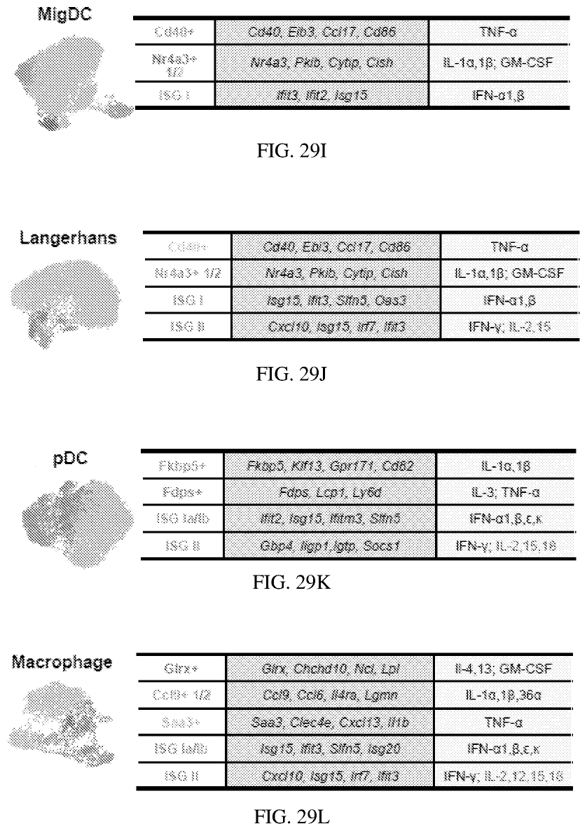

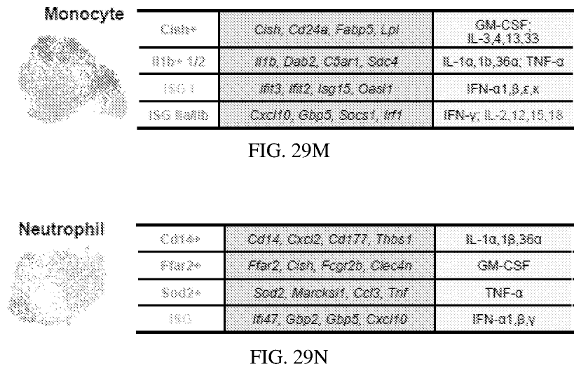

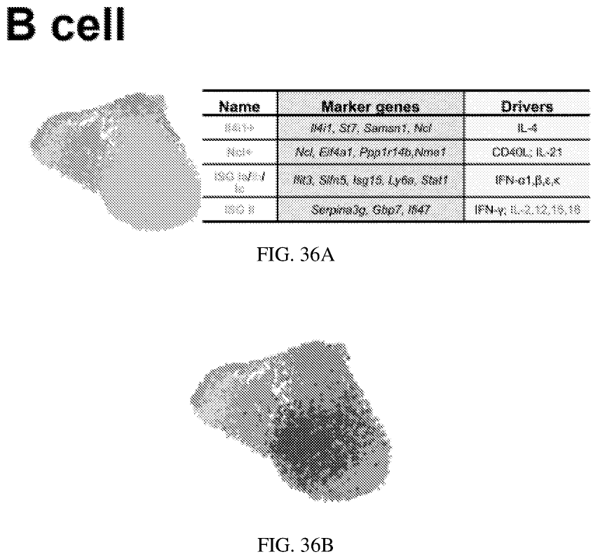

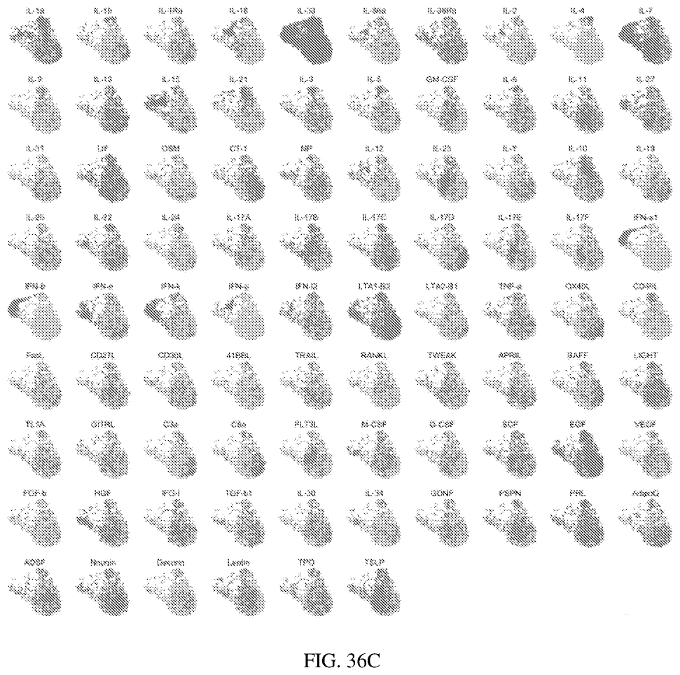

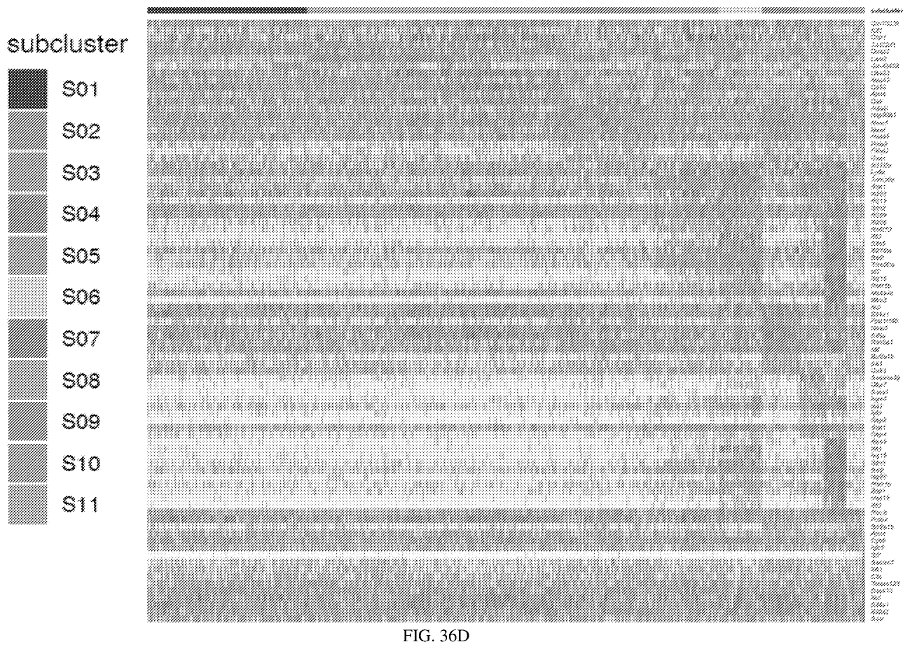

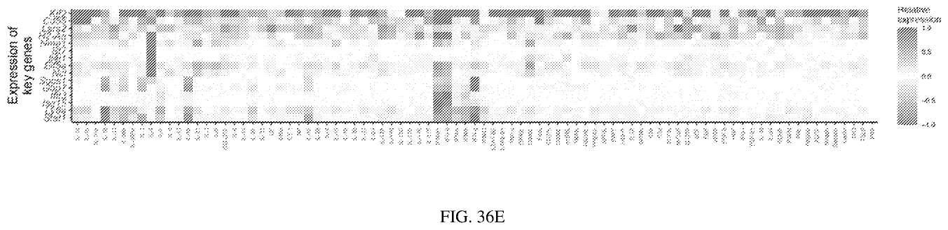

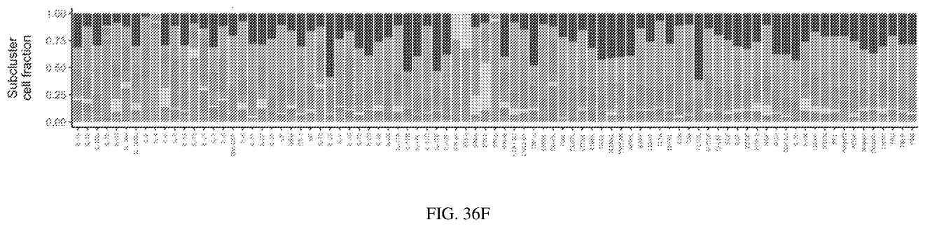

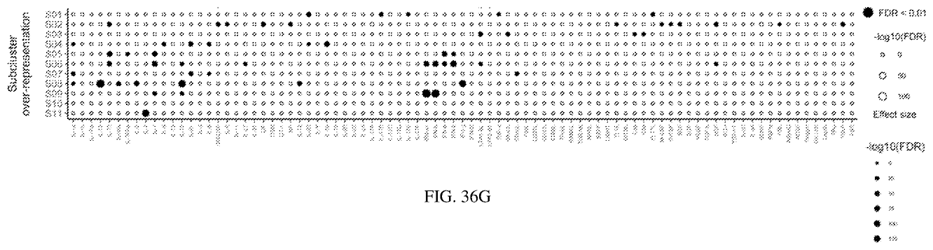

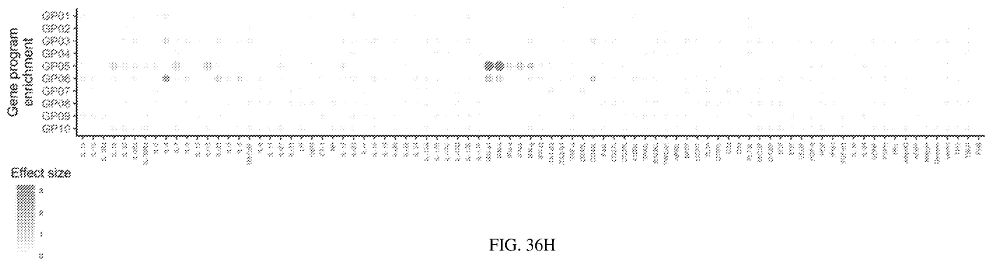

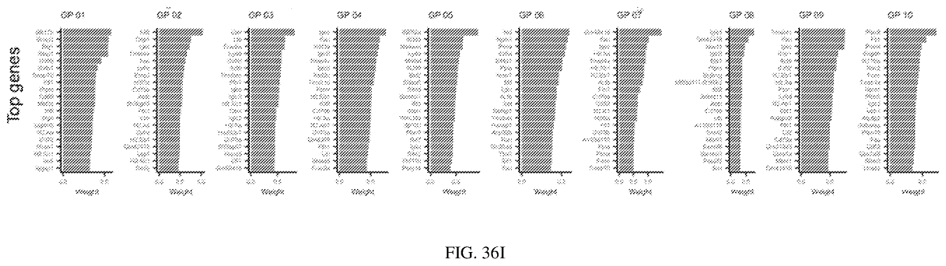

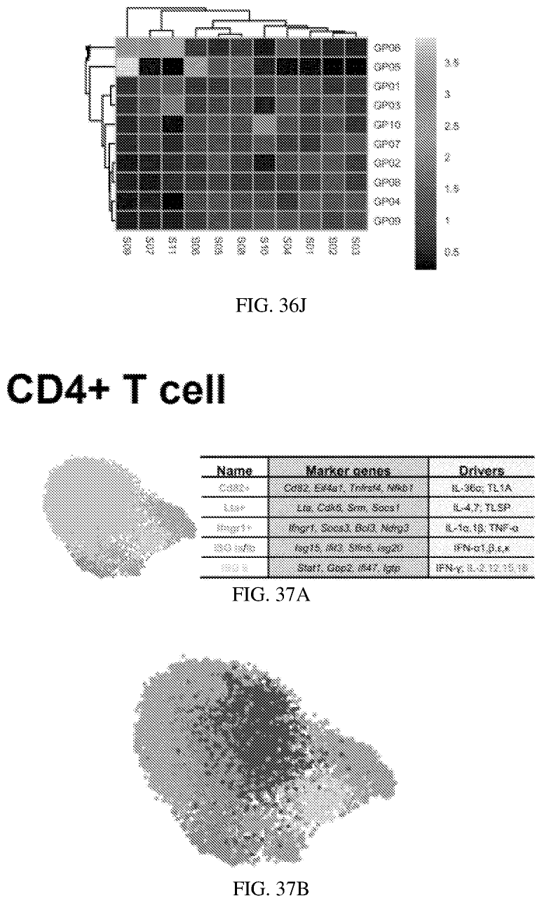

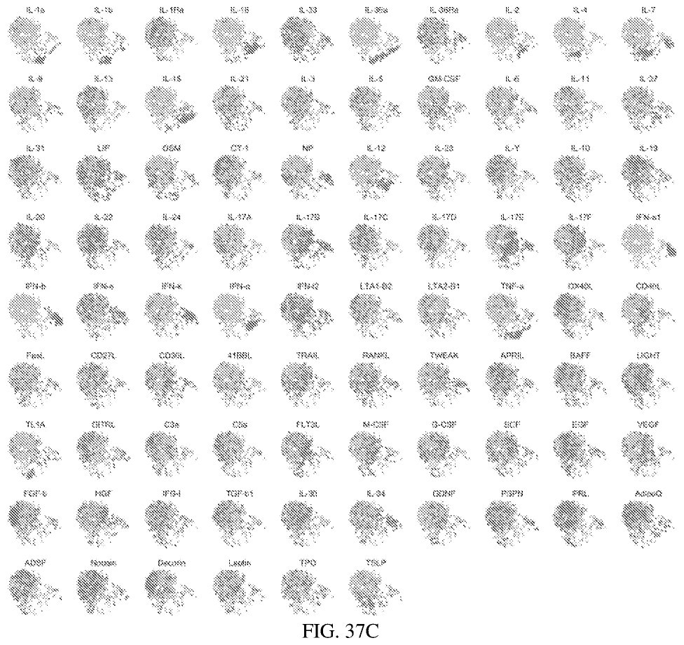

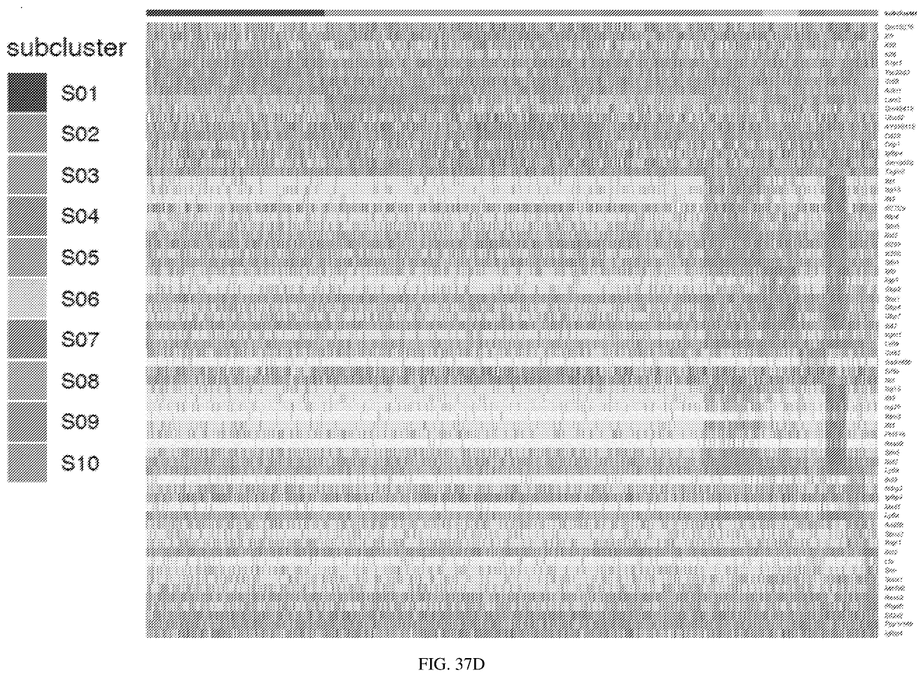

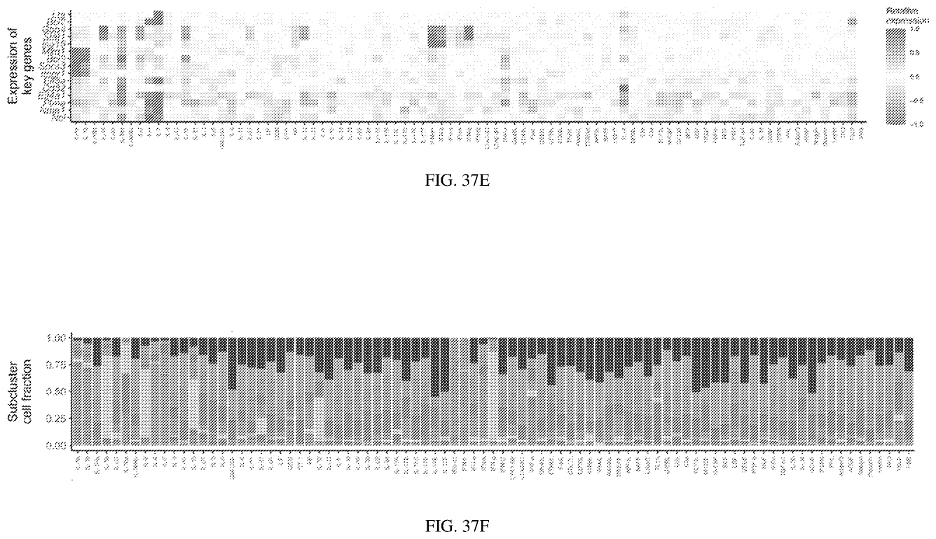

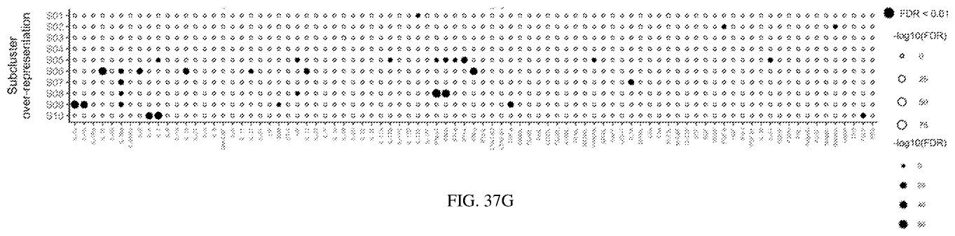

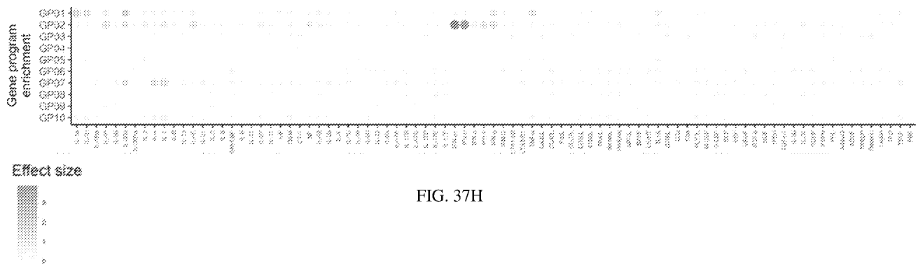

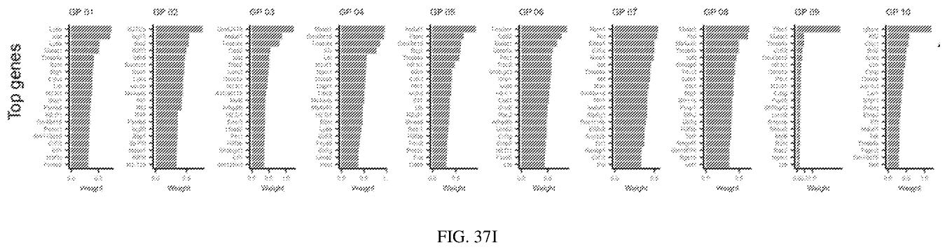

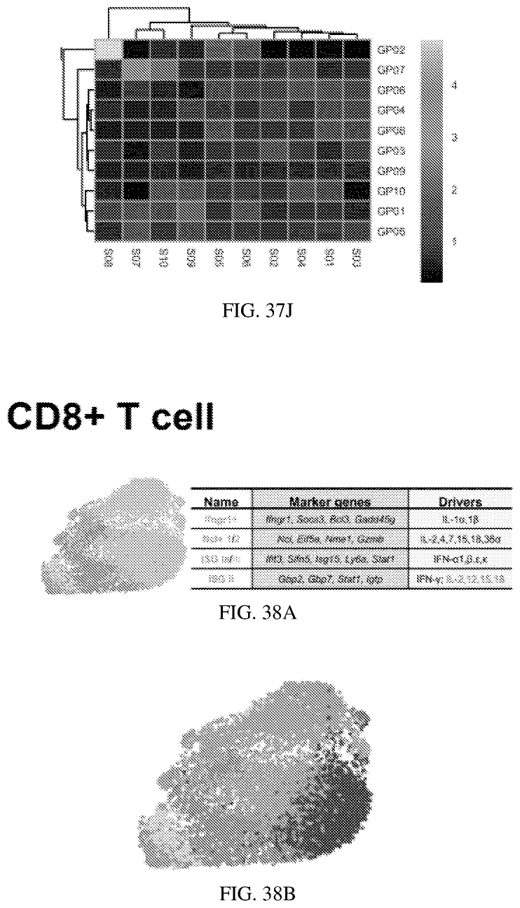

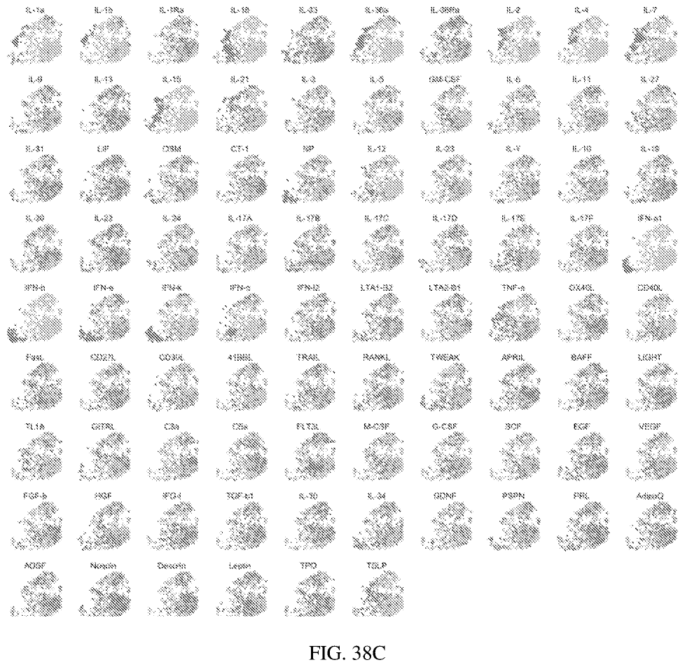

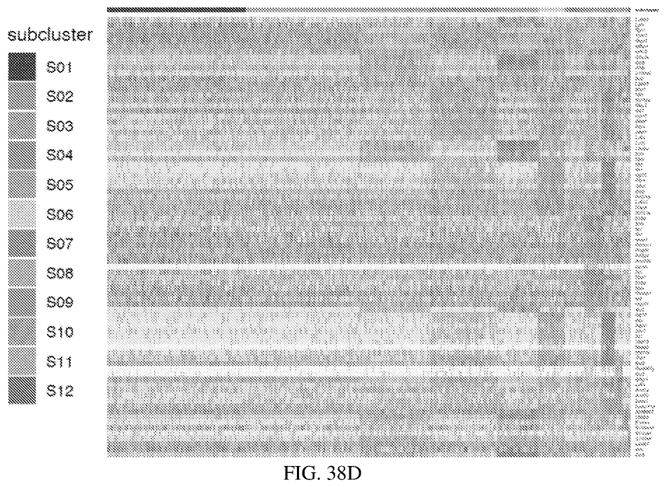

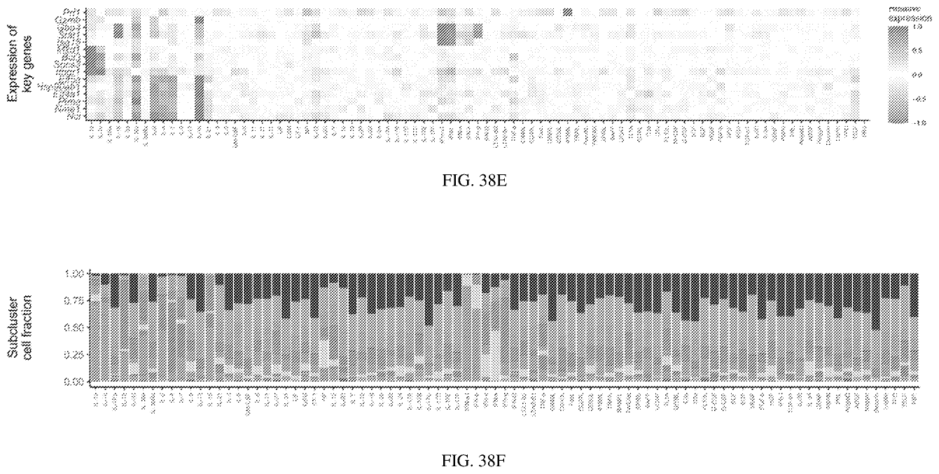

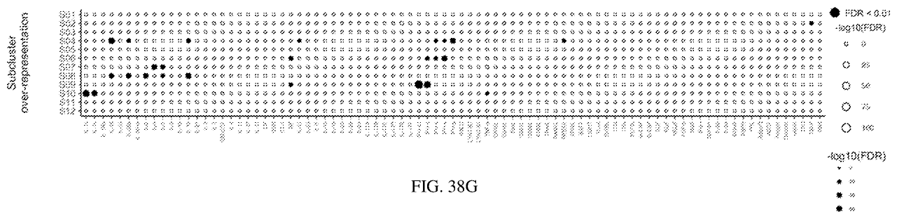

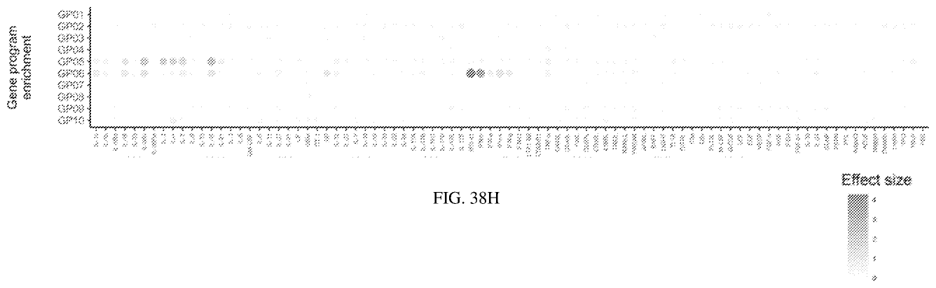

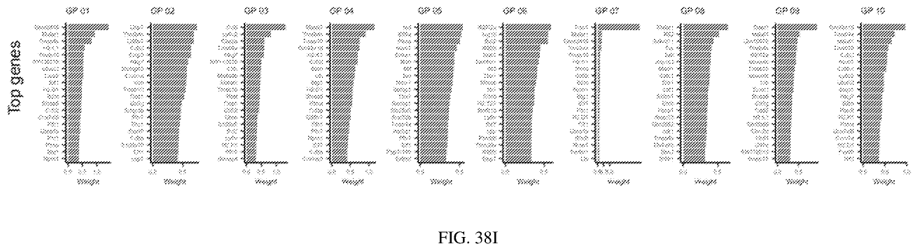

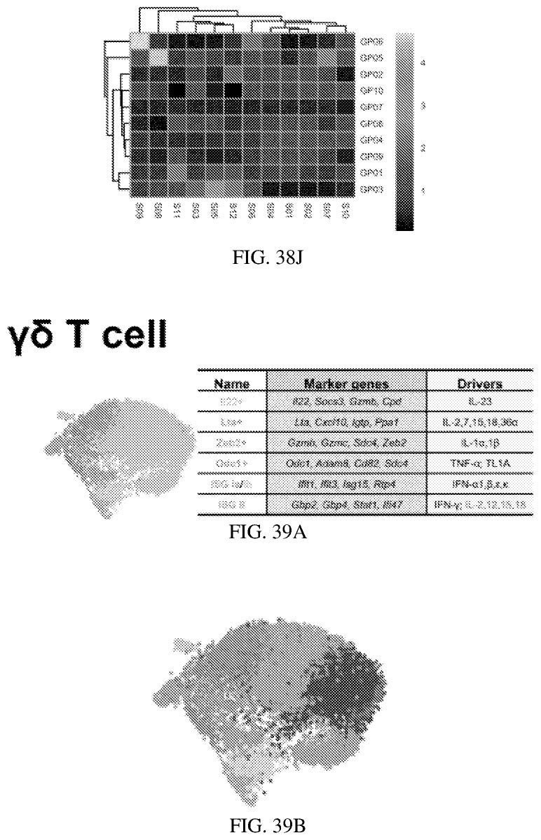

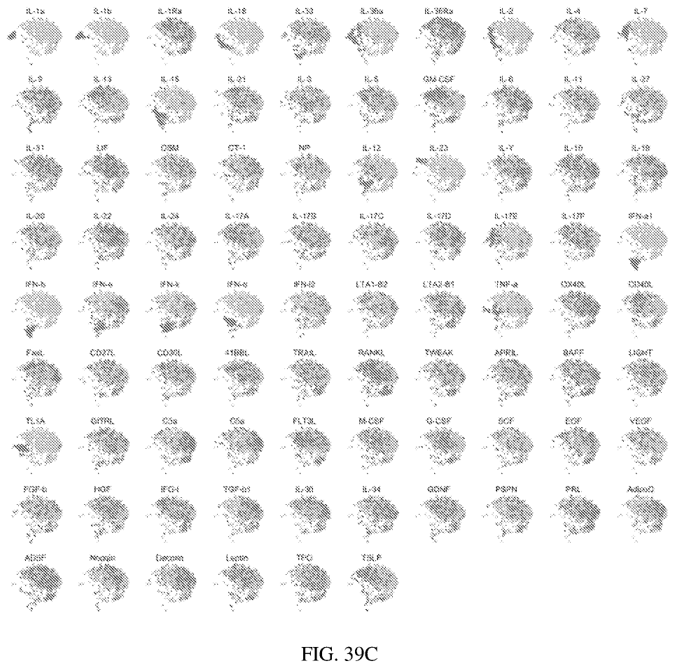

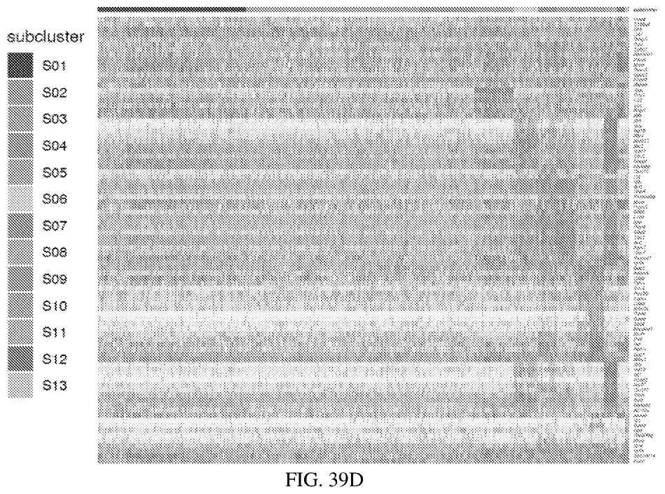

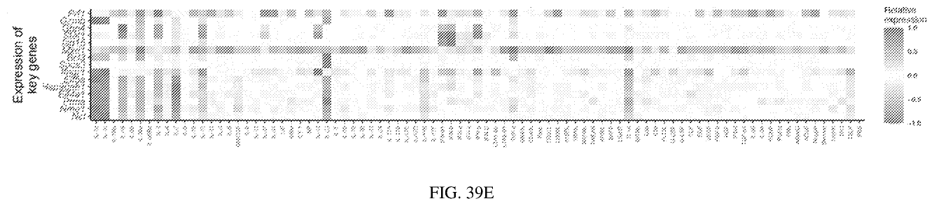

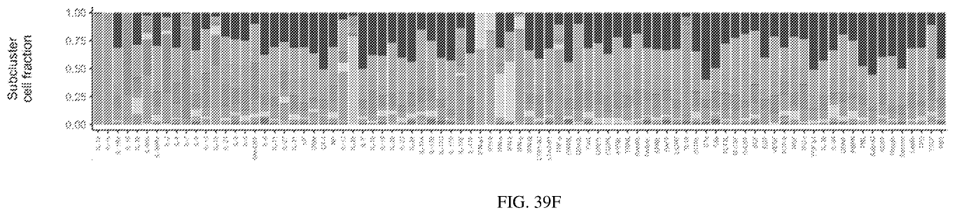

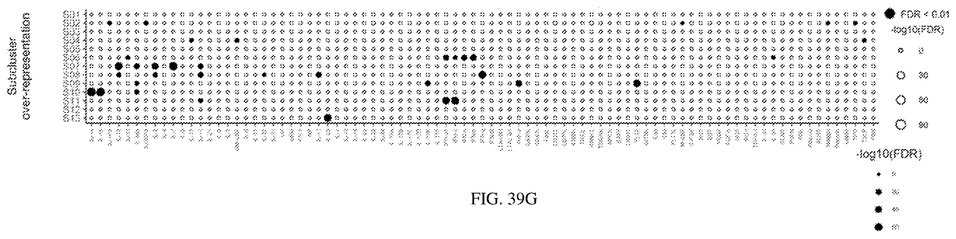

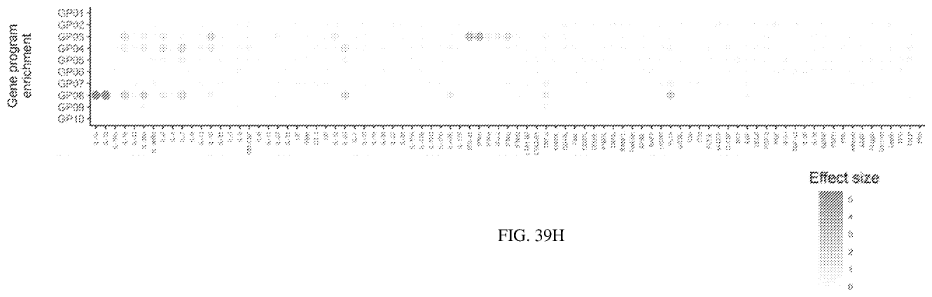

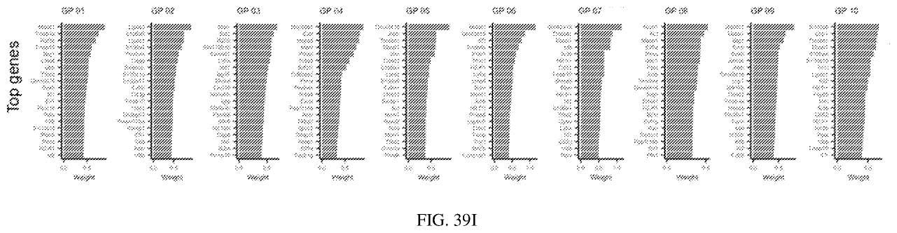

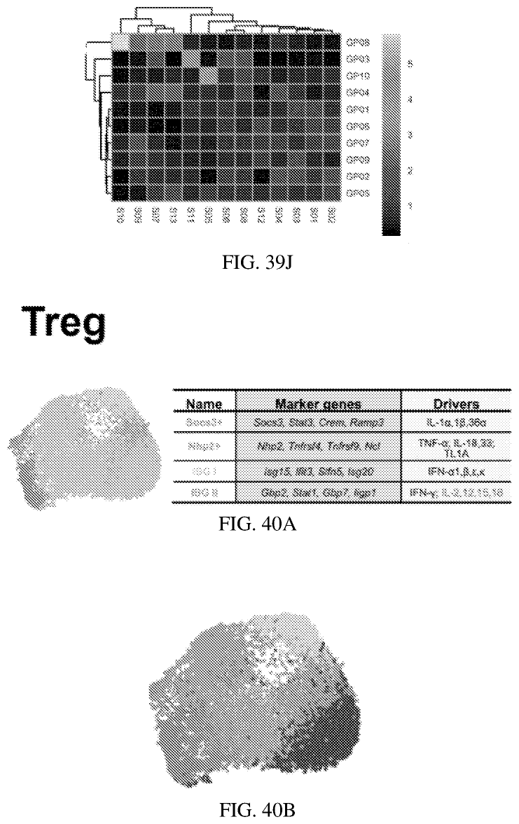

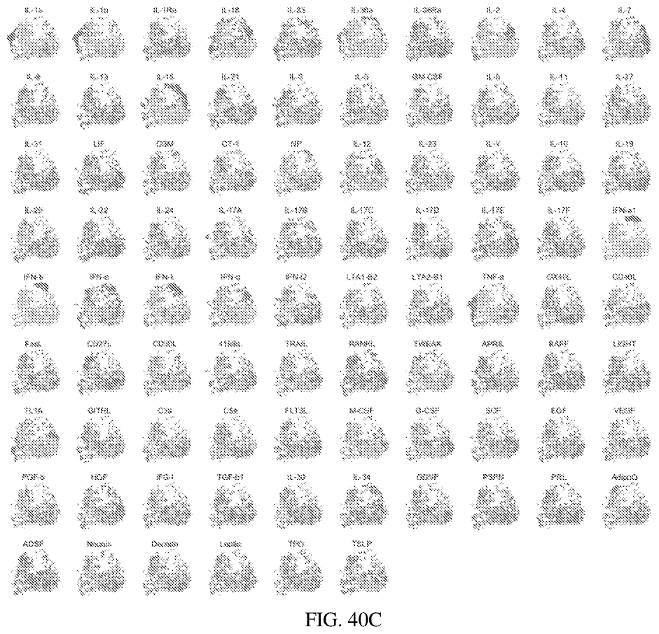

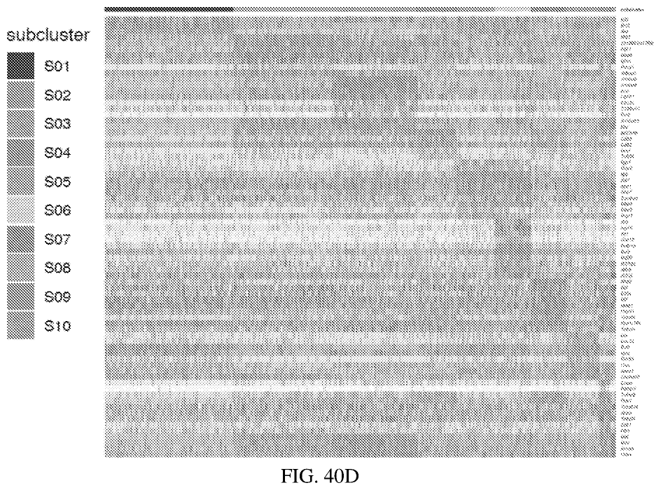

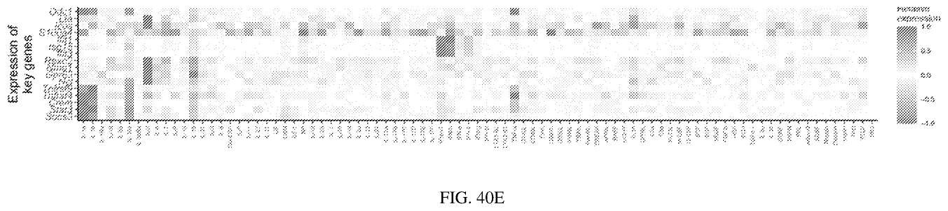

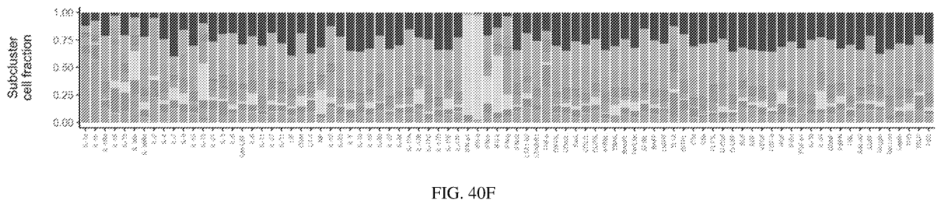

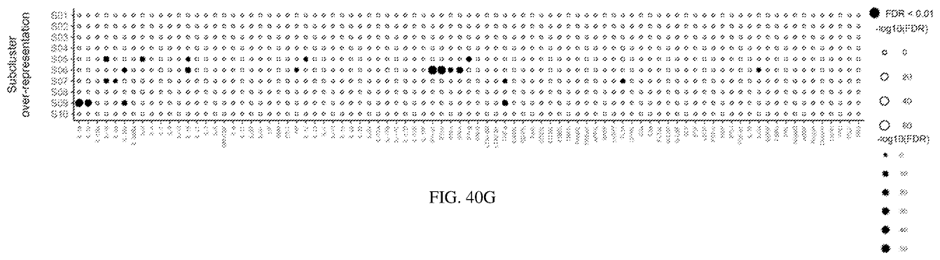

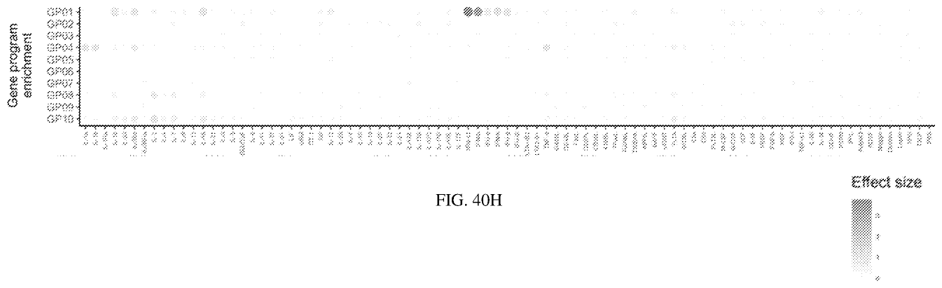

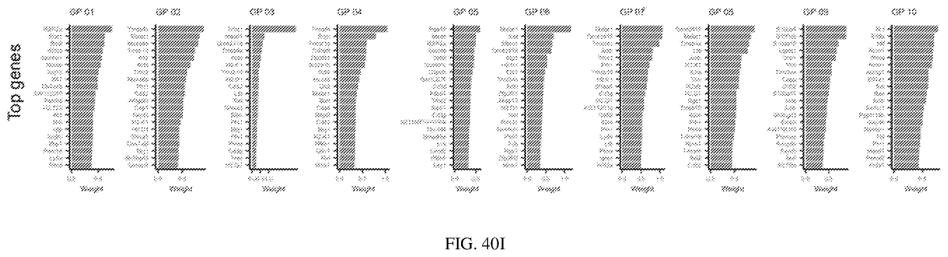

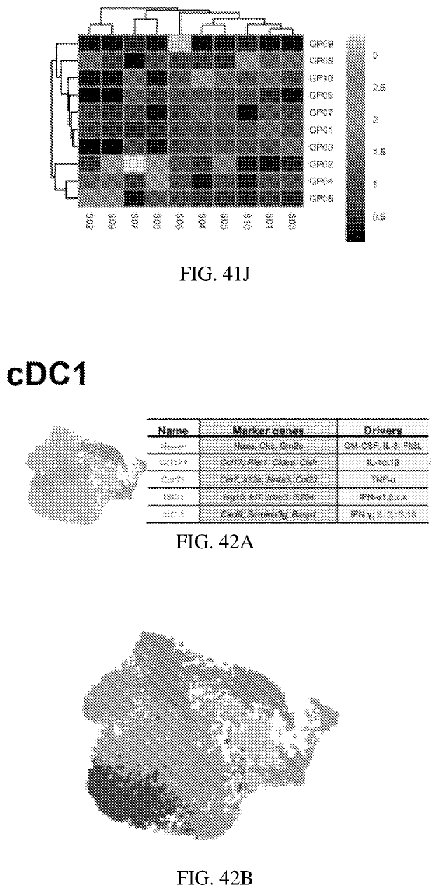

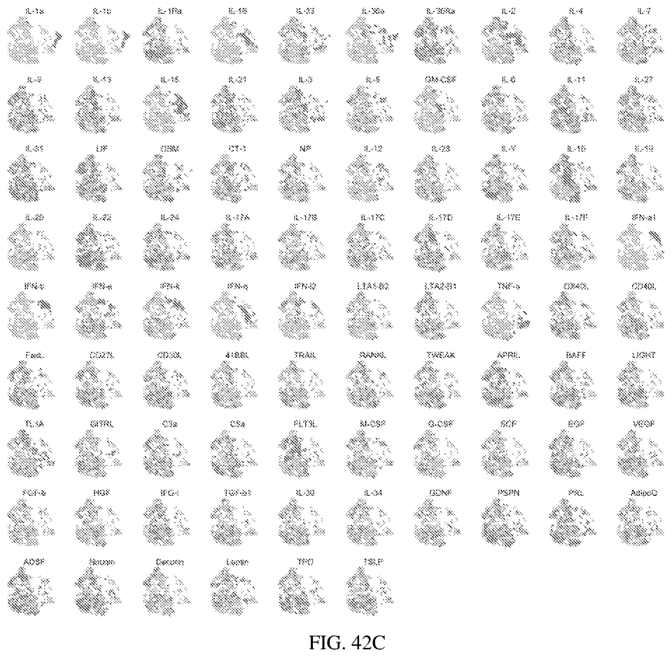

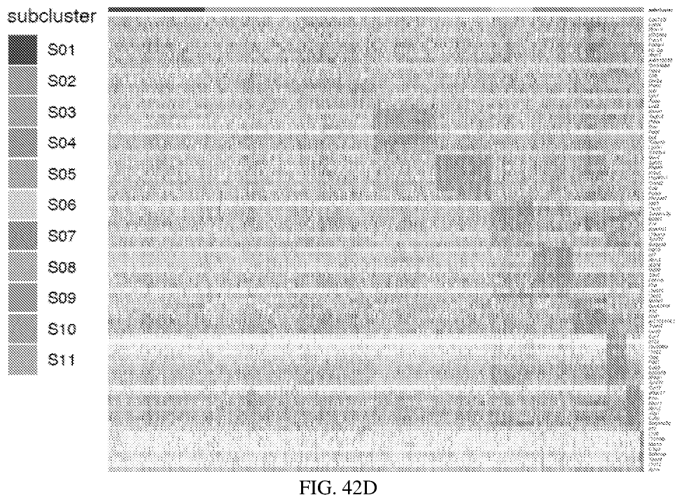

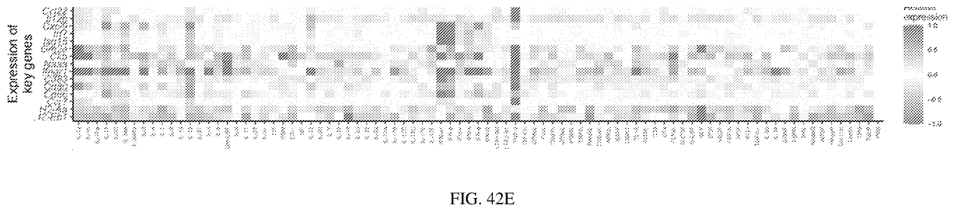

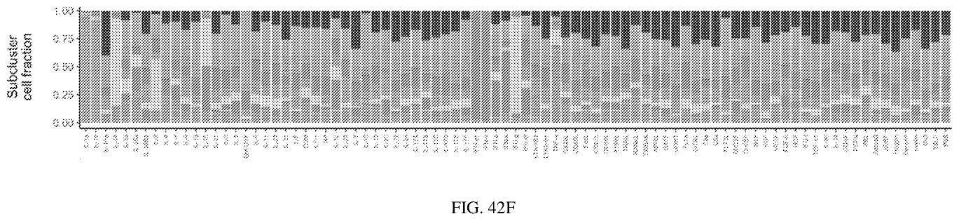

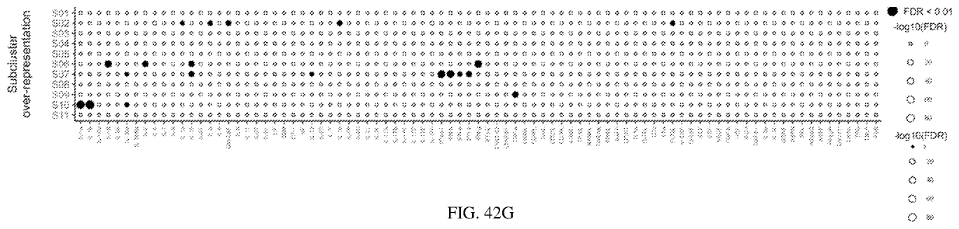

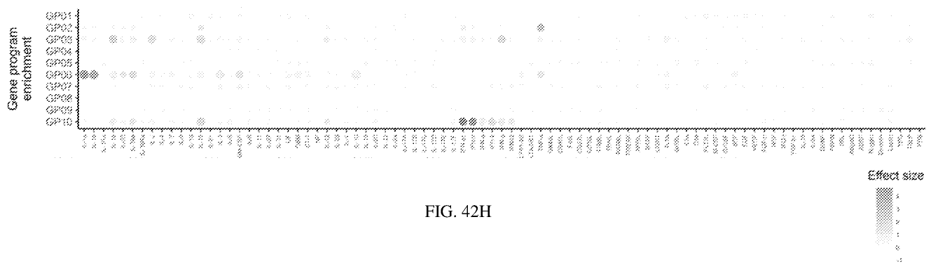

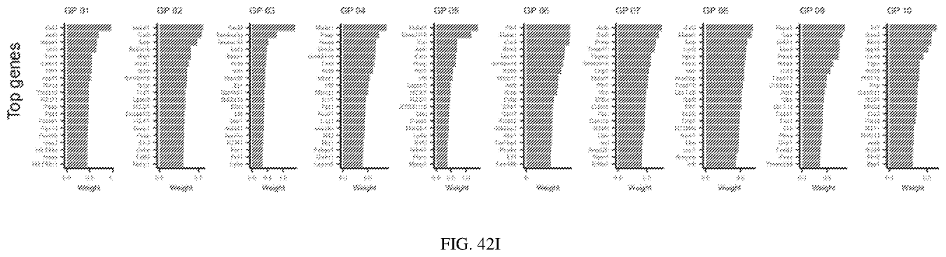

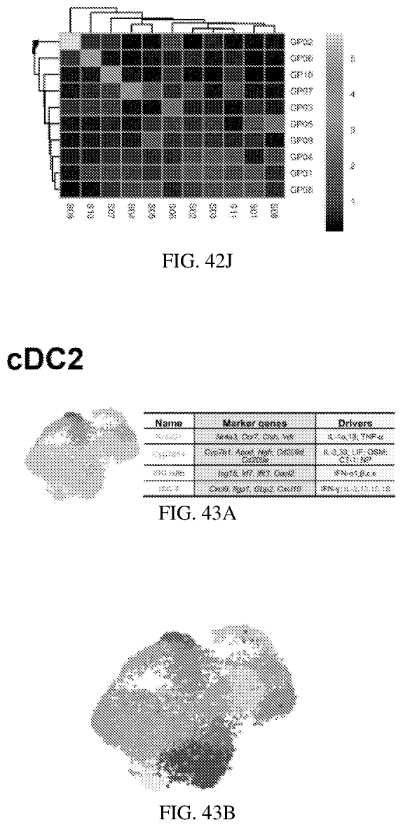

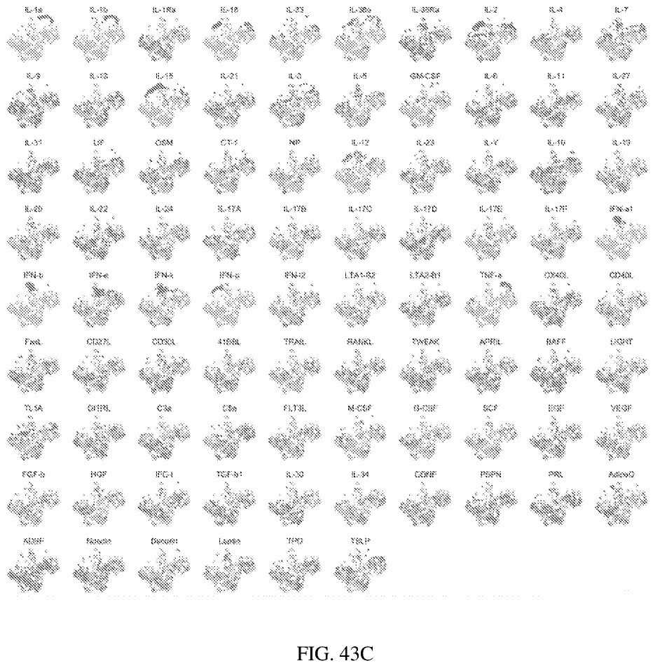

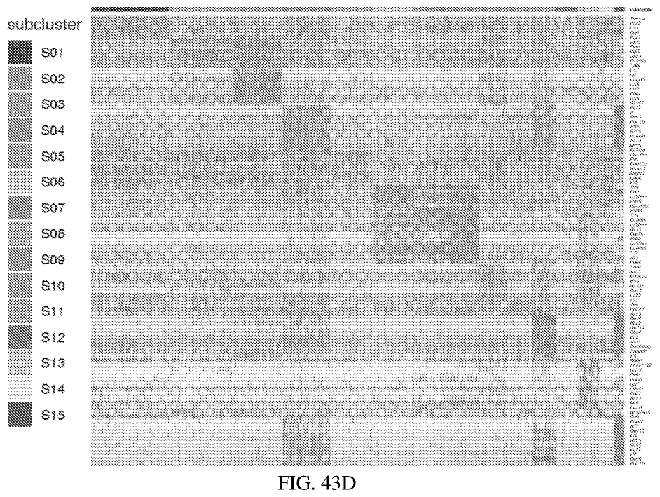

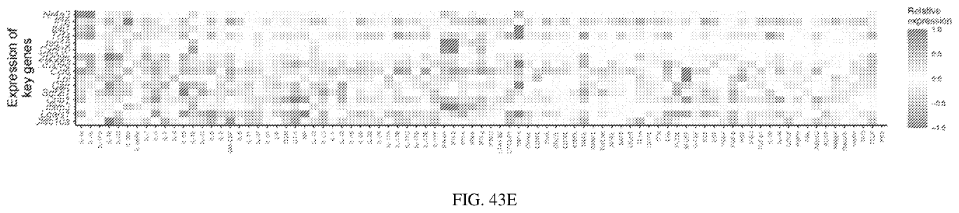

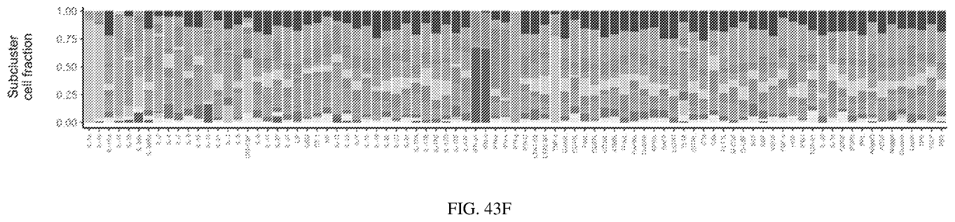

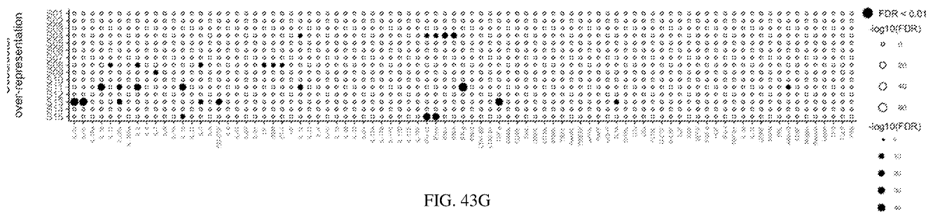

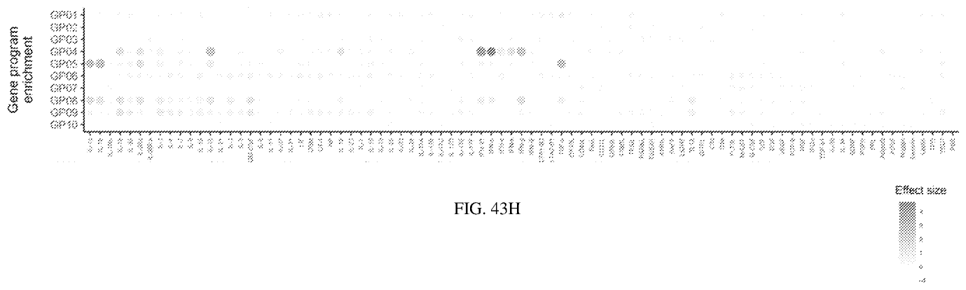

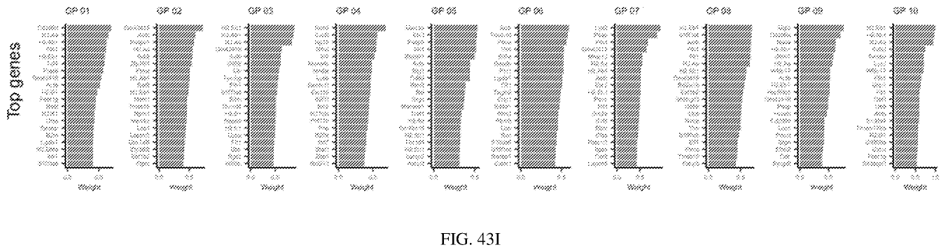

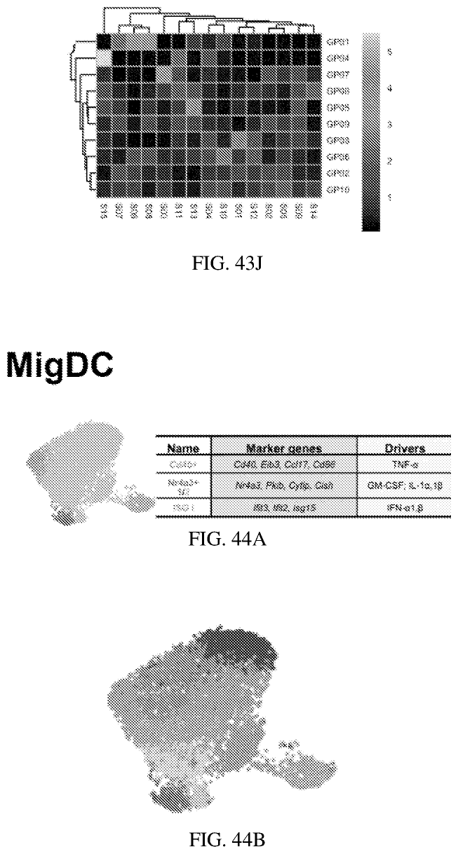

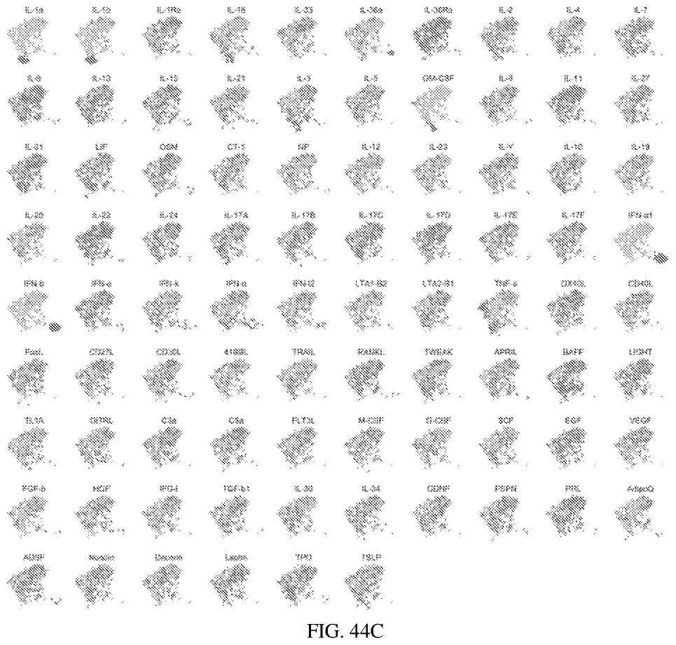

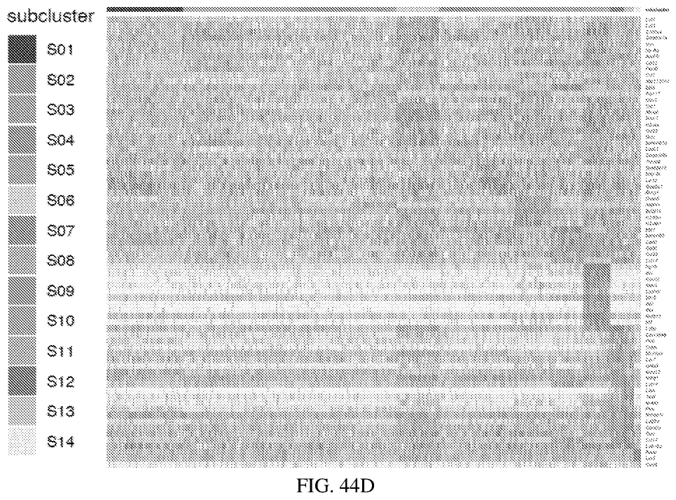

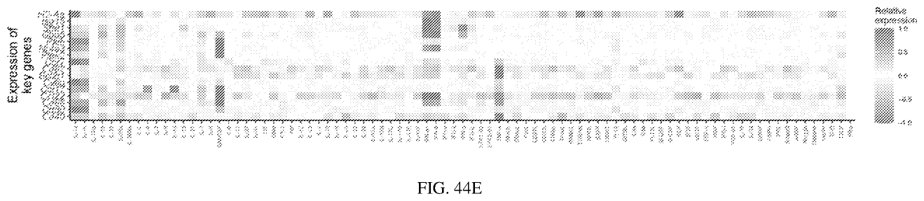

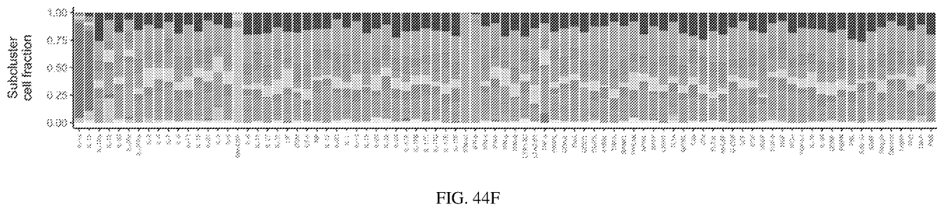

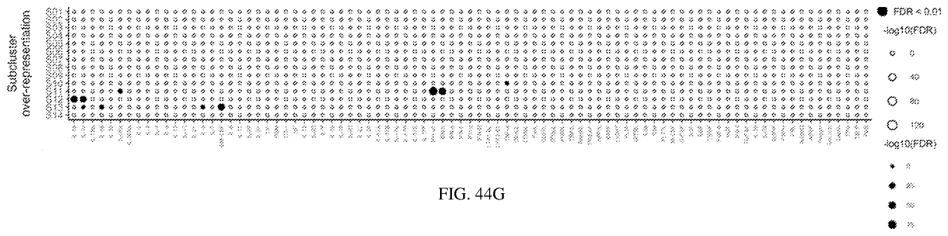

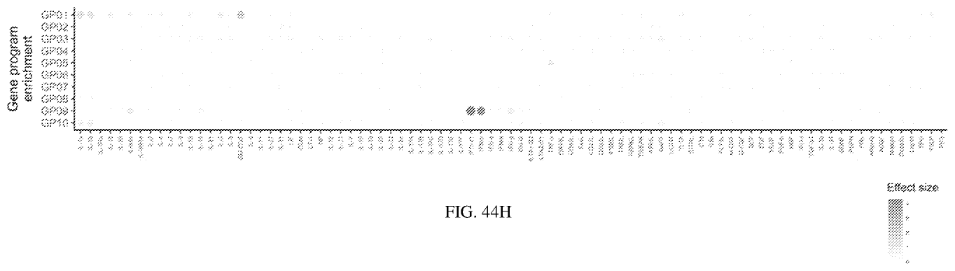

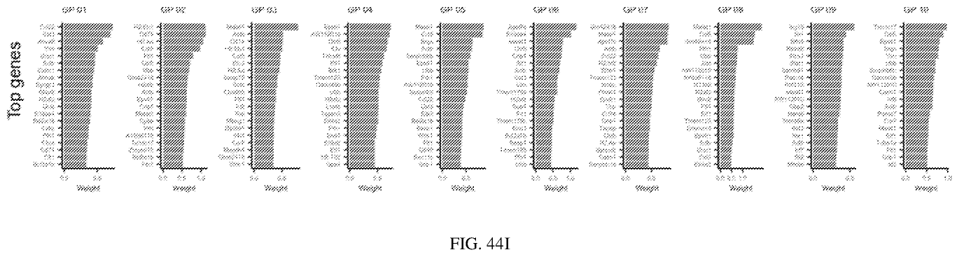

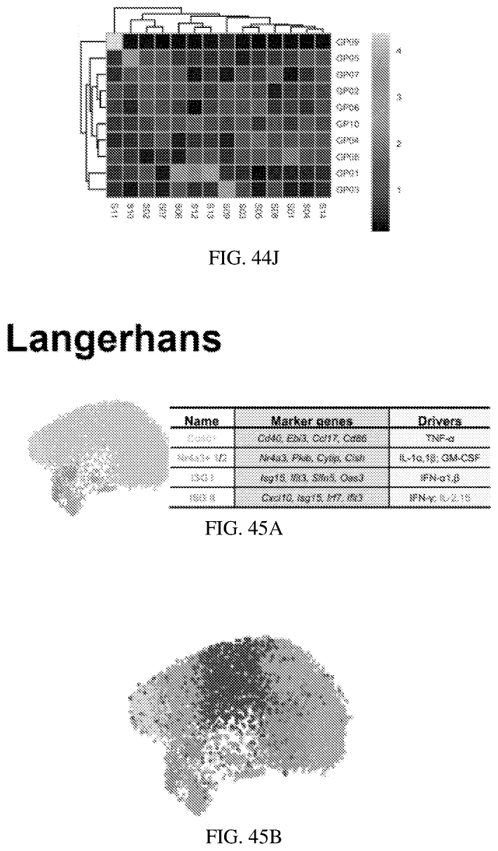

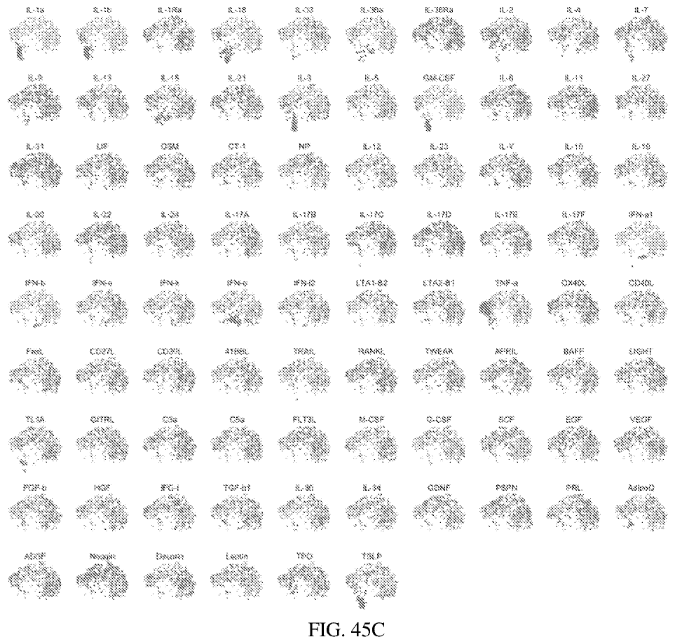

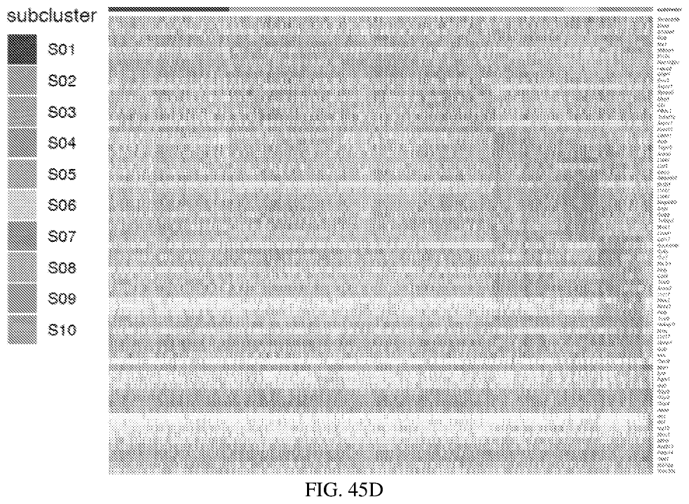

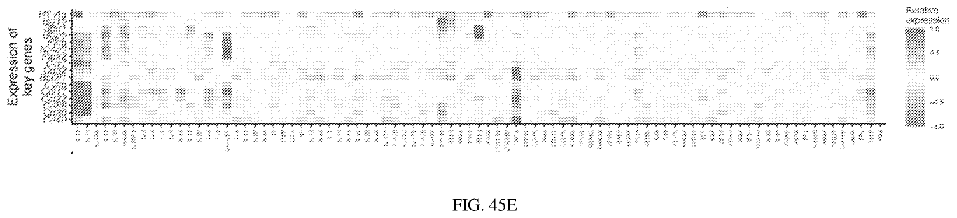

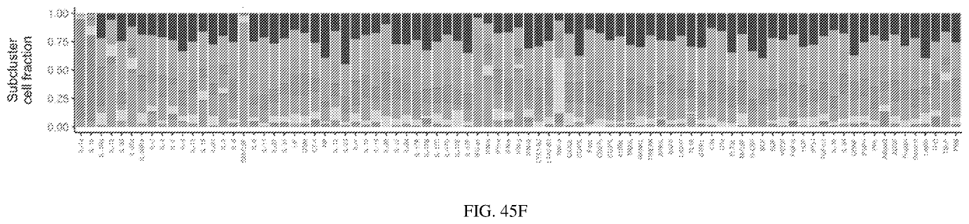

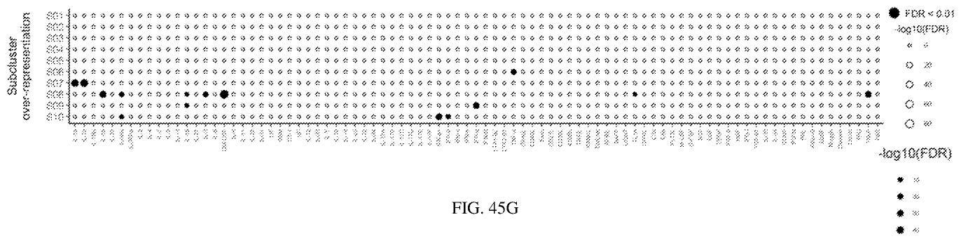

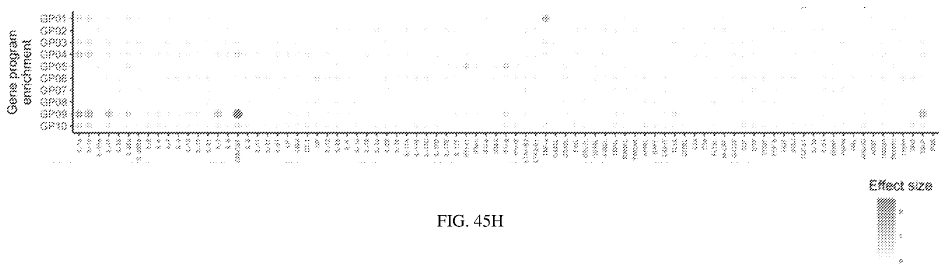

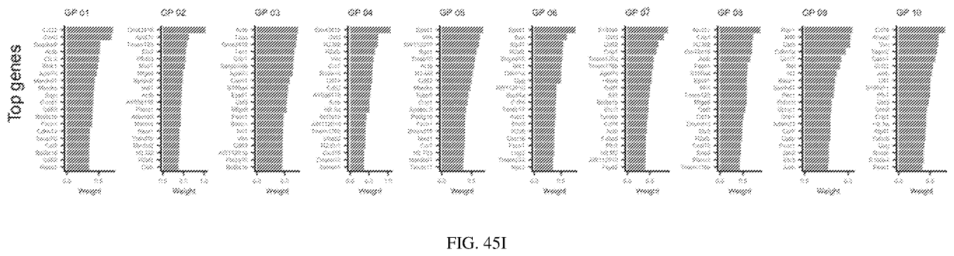

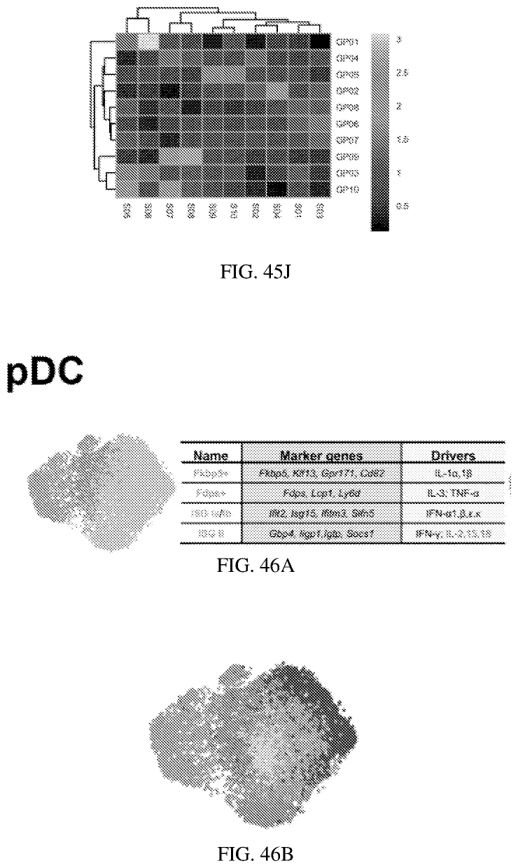

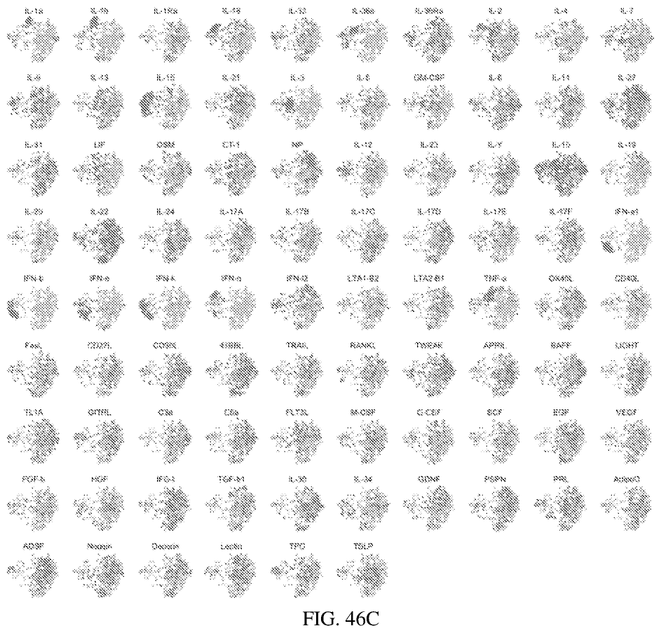

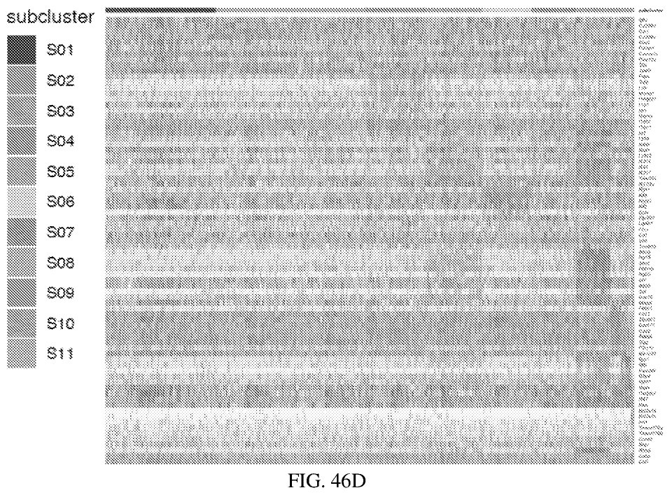

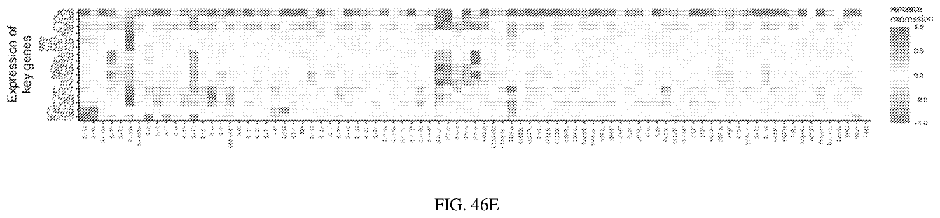

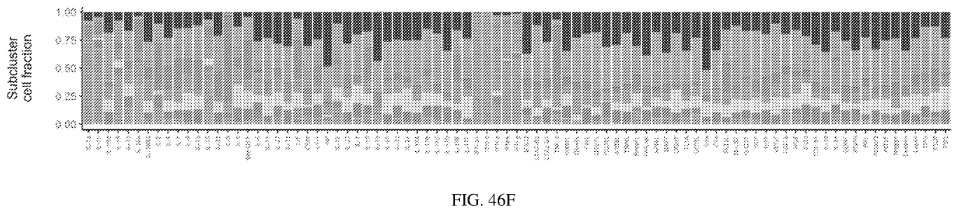

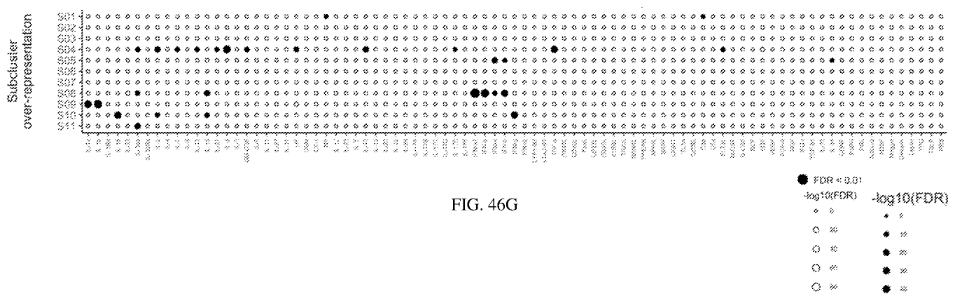

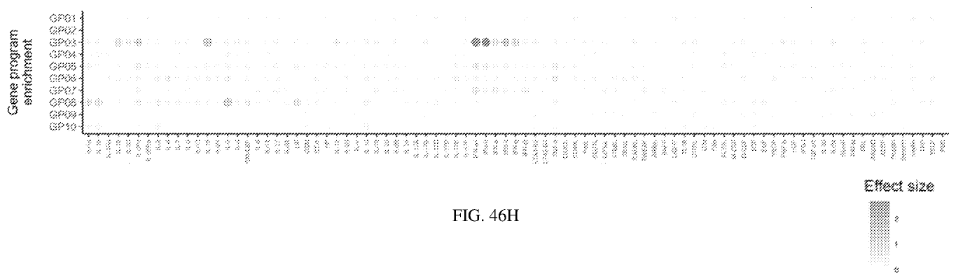

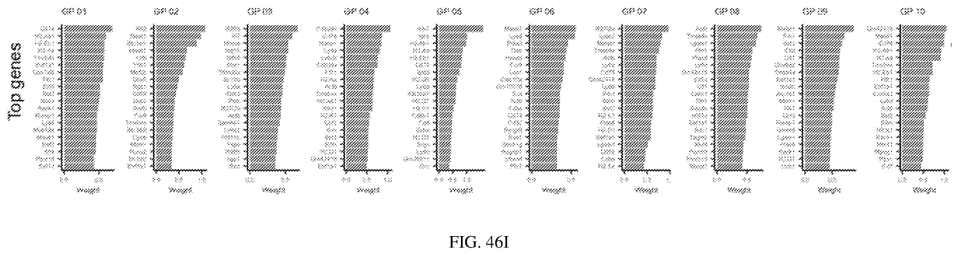

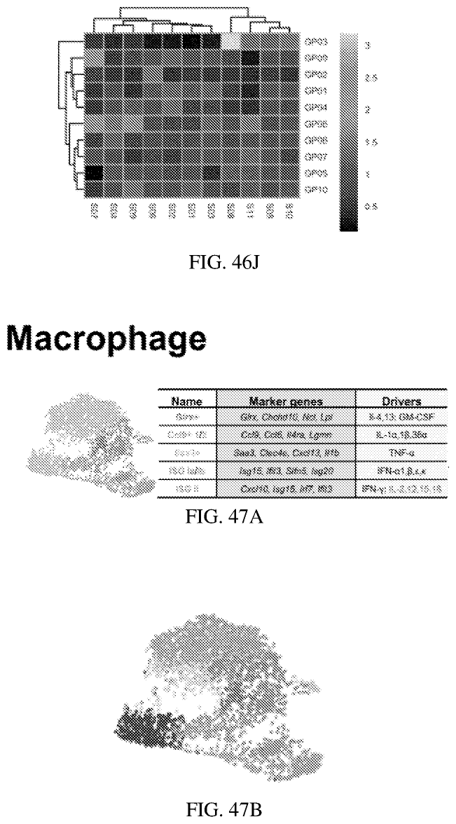

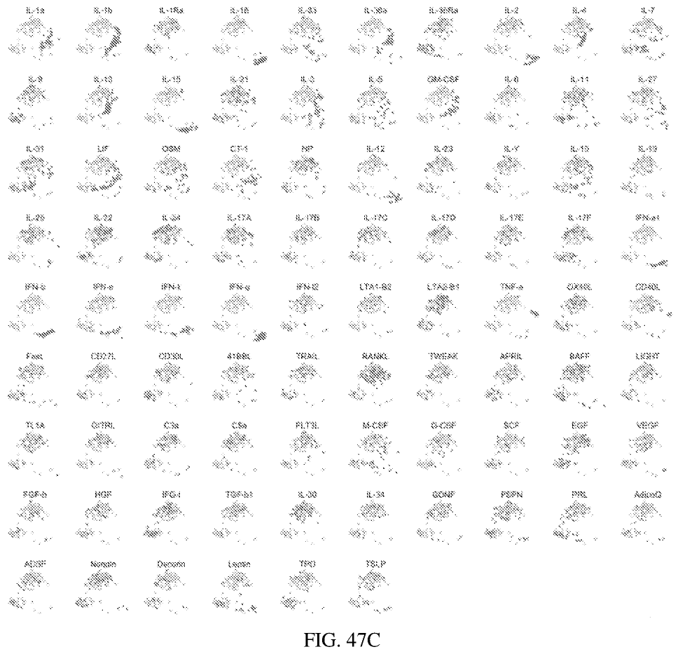

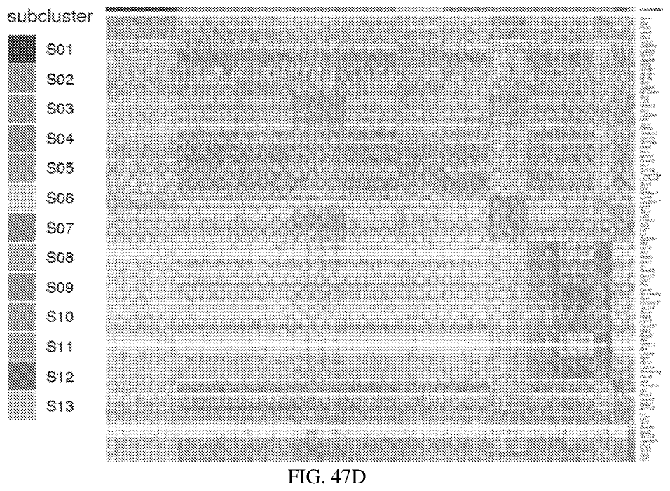

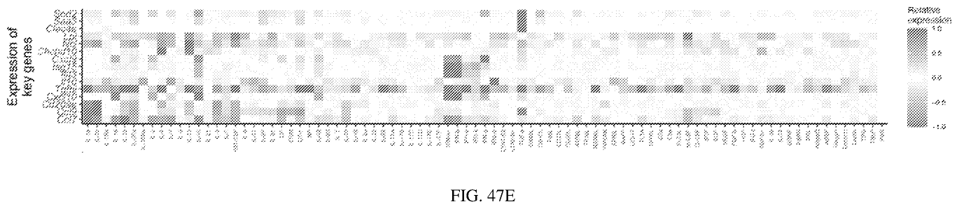

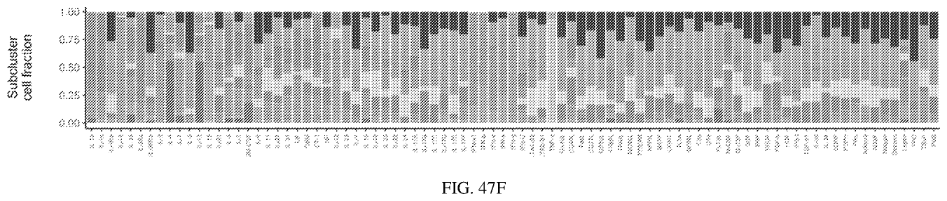

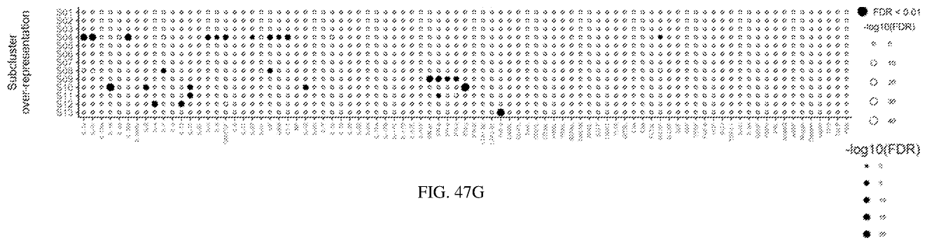

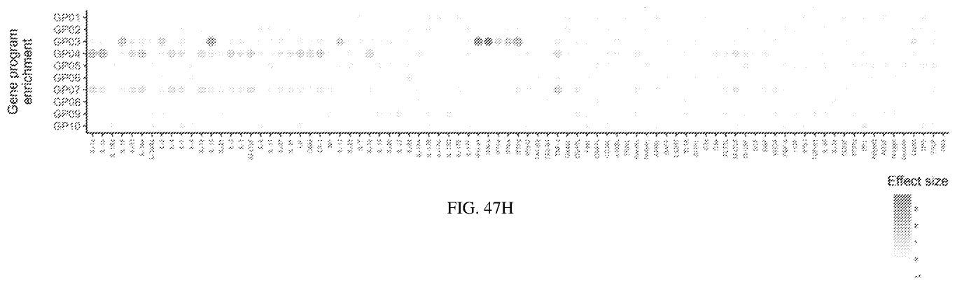

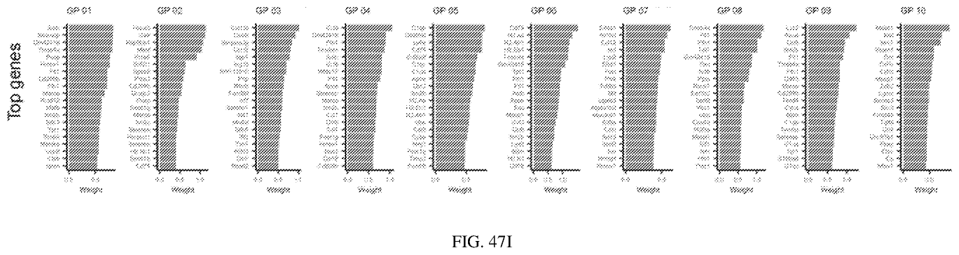

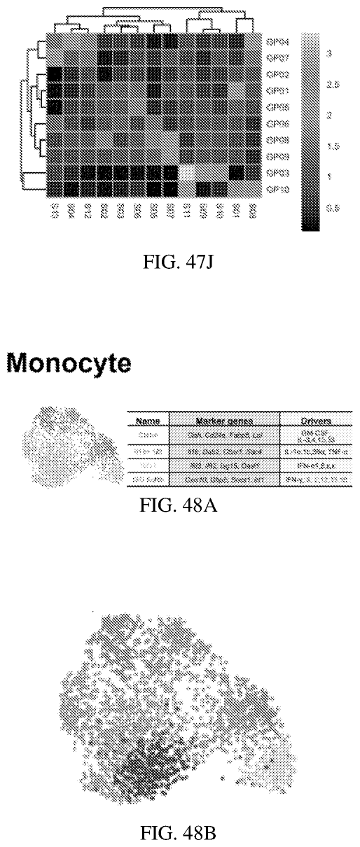

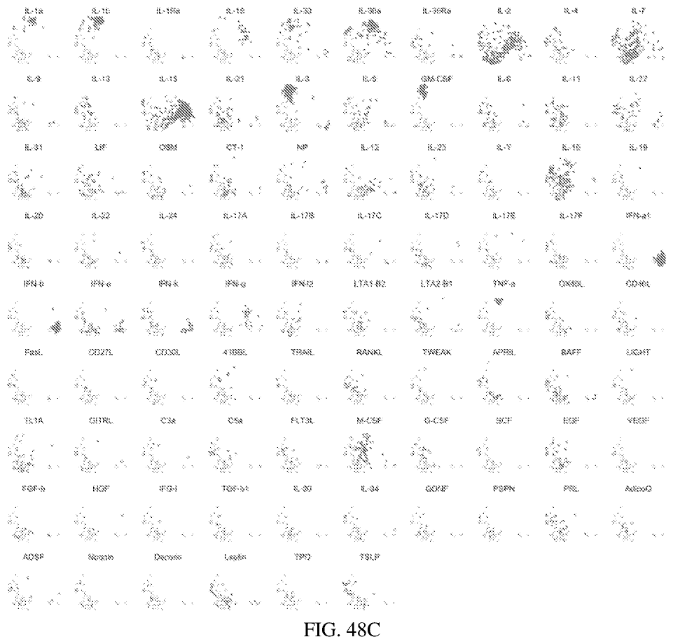

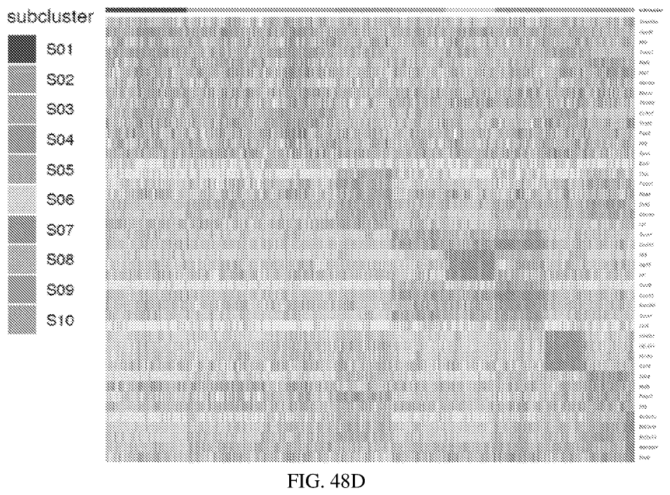

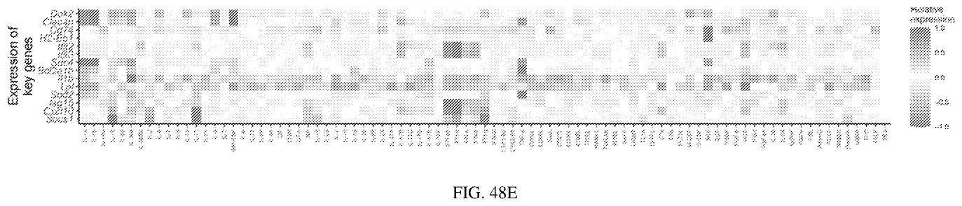

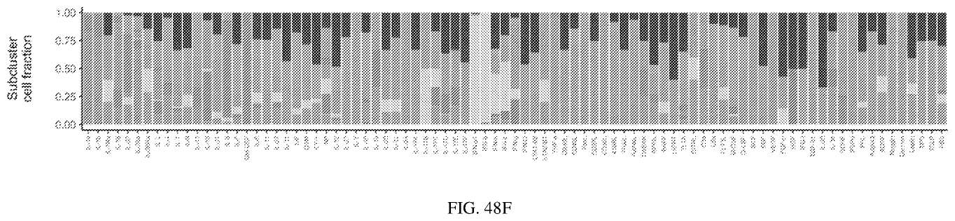

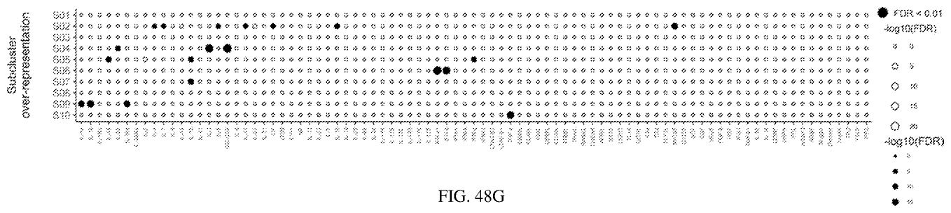

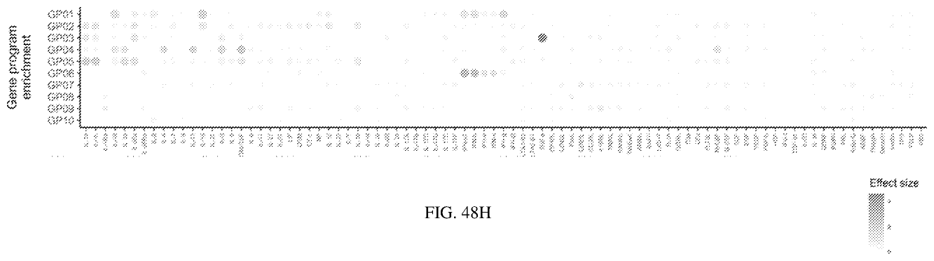

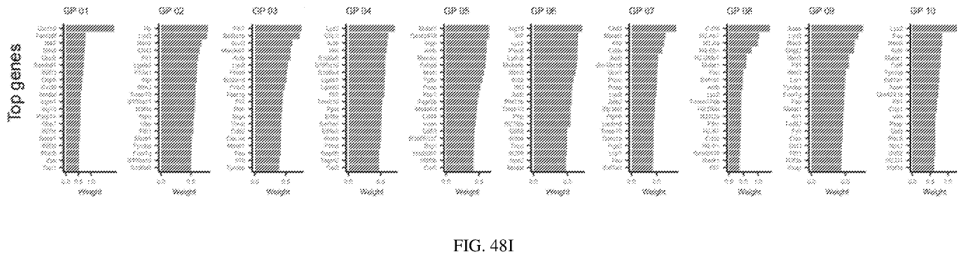

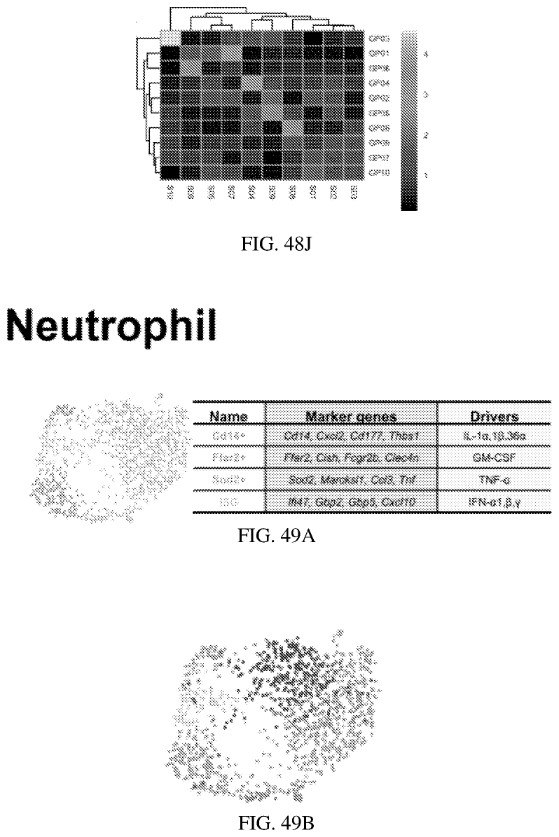

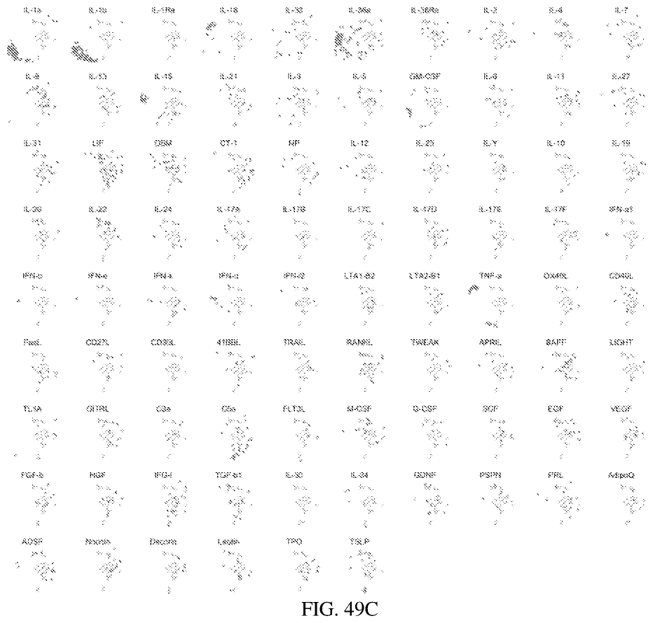

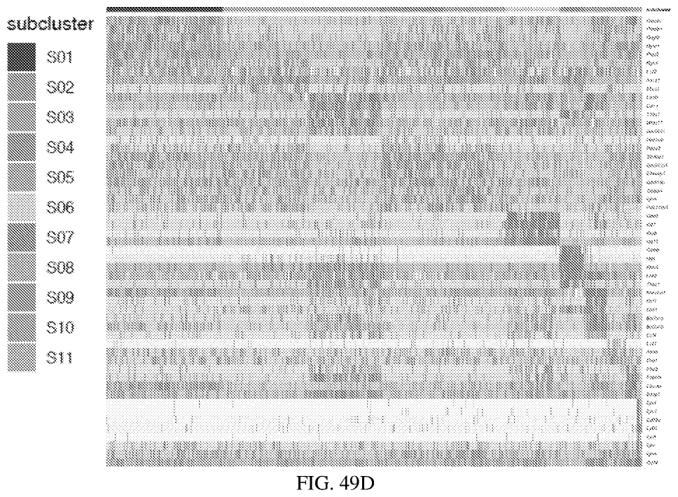

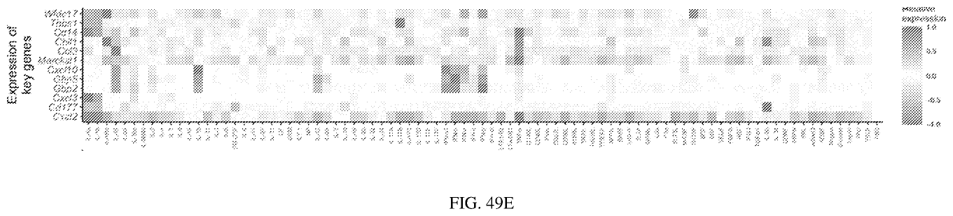

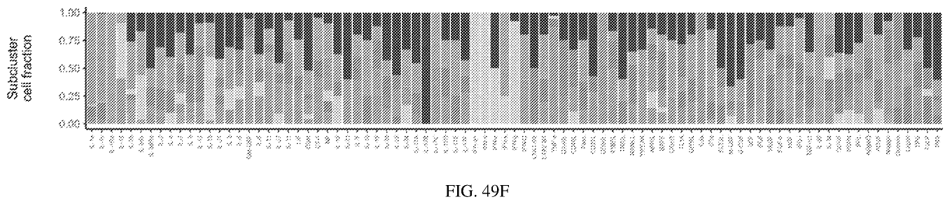

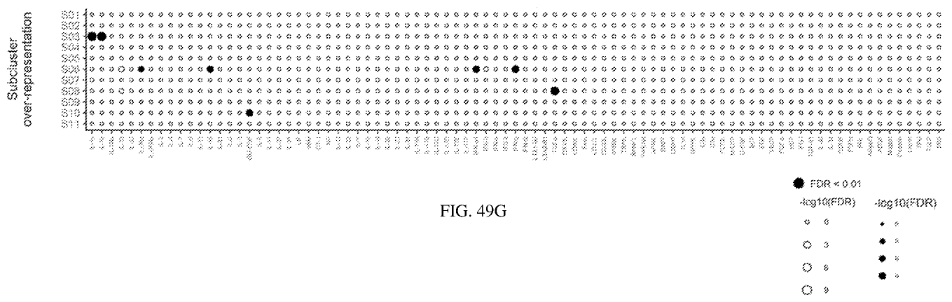

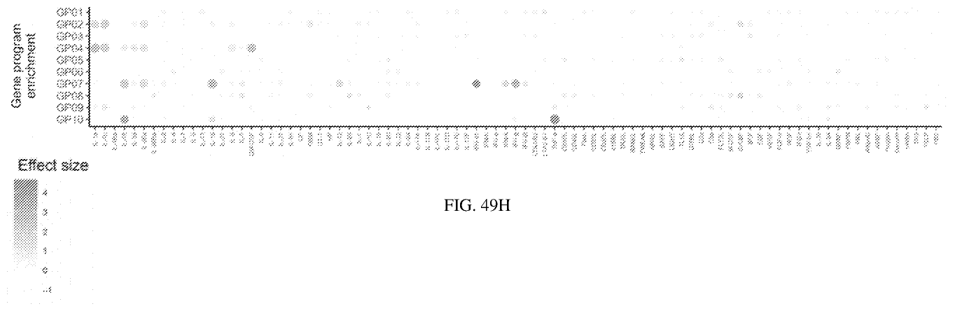

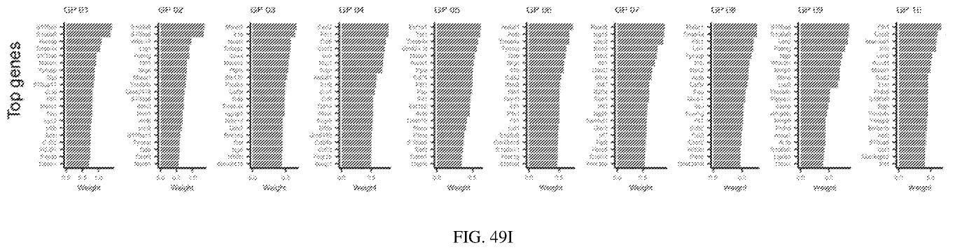

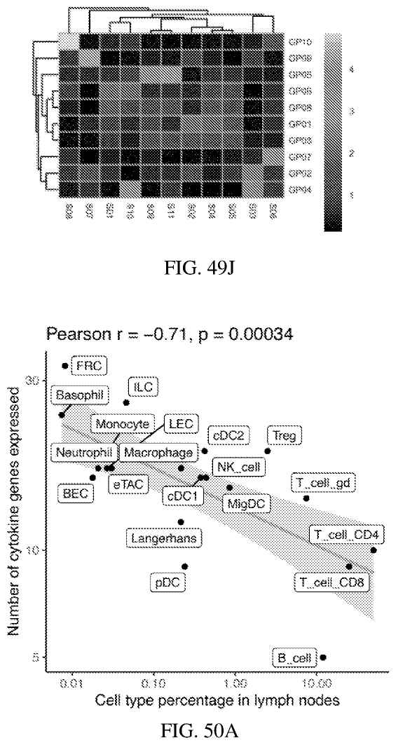

The dictionary was next used to create a cell type-centric map of cytokine-induced transcriptomic responses. Cytokines are major drivers for cellular polarization, with a classic example being cytokines driving M1-like and M2-like macrophages²⁵. Gene expression studies have been particularly useful for defining cellular states, and have uncovered cell states in different environmental cues^{12,26-28}. However, the polarization states of many cell types responding to different stimuli are yet to be comprehensively characterized. To systematically identify single-cytokine-induced cellular polarization states, sub-clustering of each major immune cell type²⁹ was performed, and subclusters that were enriched for cytokine-treated cells compared to PBS-treated control cells were identified, which are referred to as polarization states (major states summarized in FIG. 29, complete landscape in FIG. 36-49). For ease of reference, each polarization state was named by one or a group of genes upregulated in the state.

When macrophages and monocytes were examined to see if this approach could uncover previously established polarization states (FIG. 29L and FIG. 29M), it was found that, as expected from prior studies¹², IFN-γ induced M1-associated pro-inflammatory genes (e.g., Cxcl9, Cxcl10), IL-4 and IL-13 induced a distinct state that did not include these pro-inflammatory genes, and TNF triggered a third unique polarization state. It was also confirmed that monocytes turned on more Il1b when treated with IL-1a or IL-10, suggesting a role for IL-1 in an inflammatory feed-forward loop 3°.

NK cells are known for their cytotoxic functions, but other functions are still being discovered³¹. As expected, IL-2, -7, -12 and -15 induced a state with elevated cytotoxic genes, and type I interferons induced both cytotoxic genes and ISGs (FIG. 29F, FIG. 41). However, IL-18 induced a distinct polarization state—expressing genes involved in cytotoxicity, proliferation and attraction and differentiation of myeloid cells (e.g., GM-CSF as described above)—that was not induced by other cytokines such as IL-2 or IL-15. IL-1α/β did not upregulate cytotoxic molecules, but rather upregulated Ifngr1 that may, in turn, enhance NK cell activation by IFN-γ. Similar to its effect on NK cells, IL-1α/β induced Ifngr1 upregulation in multiple T cell subsets. While lymphocyte differentiation states have been studied mostly after TCR stimulation, these findings demonstrate that resting lymph node B and T cells can also be polarized by cytokines.

Shared polarization states and cytokine drivers were observed in MigDCs and Langerhans cells (FIG. 29I, 29J, 44, 45). TNF-α uniquely increased the proportion of cells in a state marked by Cd40 and Ccl17 expression, potentially enhancing antigen presentation as well as recruiting γδ T cells and Tregs via the Ccr4-Ccl17 axis as a first line of defense. GM-CSF and IL-1 family cytokines induced the upregulation of Nr4a3, which has been found to play a key role in DC migration³². These results implicate TNF-α in triggering local inflammation and DC-T cell interactions, and the IL-1 family cytokines in boosting DC migration to prime T cell responses in lymph nodes.

In summary, the reference map of single-cytokine mediated cellular polarization states and their drivers reveals plasticity across all immune cells and demonstrates that polarization states can be specific for one cell type or shared (e.g., interferons) across cell types, and are either induced by only one cytokine (e.g., IL-23 to γδ T cells) or several cytokines (e.g., IL-1 and TNF) that lead to convergent responses.

### A Cytokine Production Map

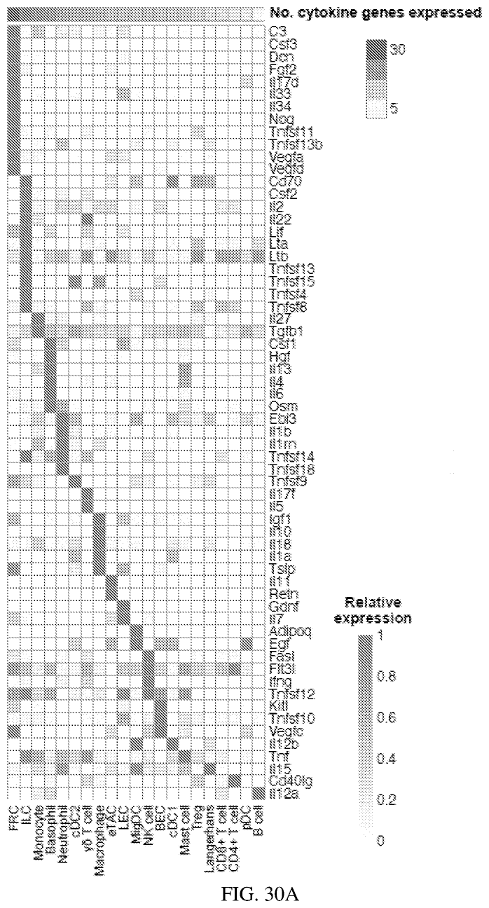

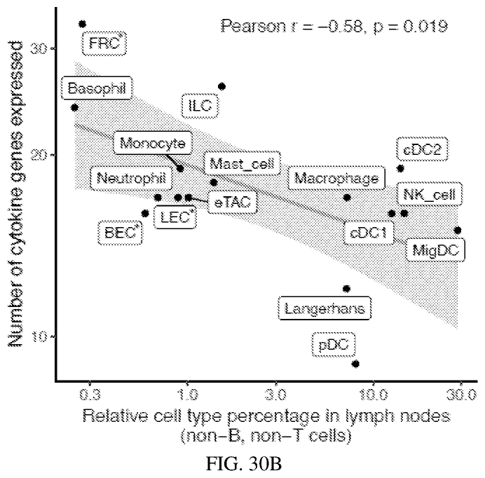

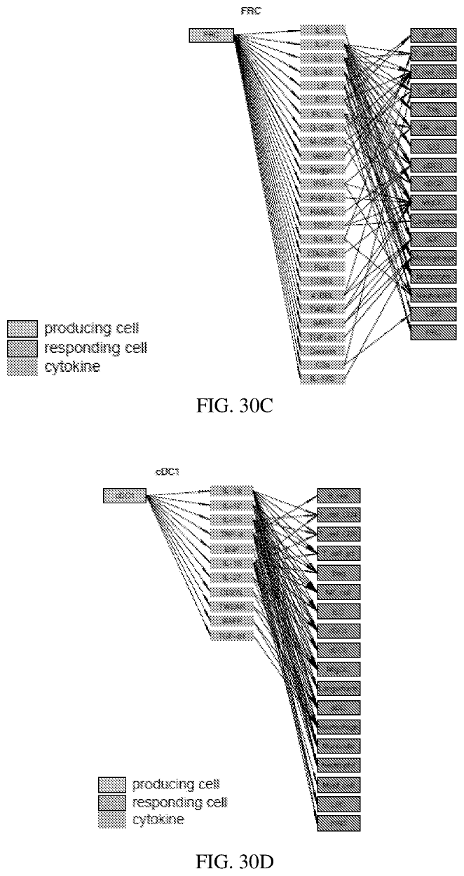

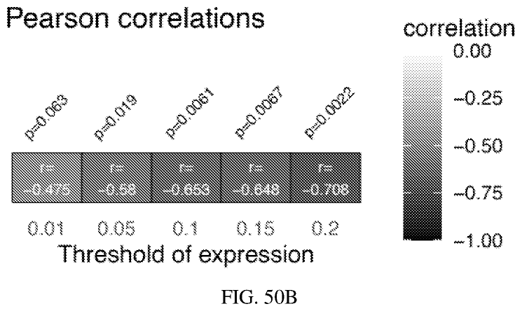

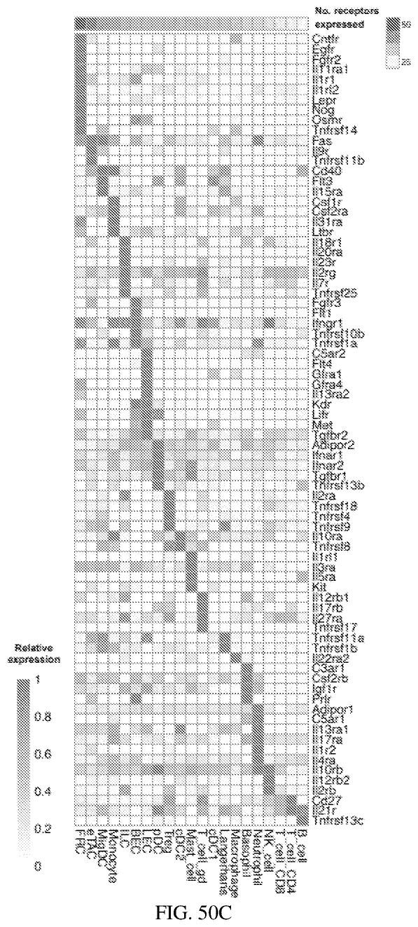

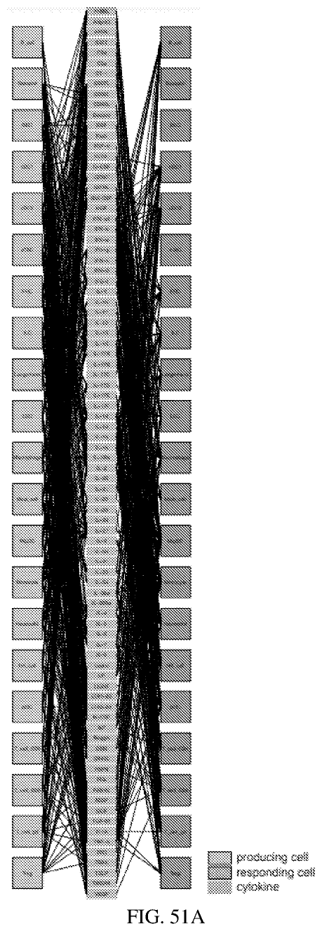

To better understand the cell-cell communication carried out by each cell type, the expression of cytokines in control or cytokine-stimulated conditions were analyzed. The majority of cytokines were found to be expressed by a small number of cell types (FIG. 30A), with FRCs expressing the highest number of distinct cytokines, consistent with prior findings with regards to their heterogeneity and maintenance functions in various immune and non-immune compartments³³. Other rarer cell types in lymph nodes, such as basophils and neutrophils, also expressed a large number of cytokines. Indeed, an inverse correlation between the number of cytokines a cell type produced and the abundance of the cell type was observed (Pearson r=−0.58, p=0.019 for non-B, non-T cells; Pearson r=−0.71, p=3e-4 for all cell types; FIG. 30B, FIG. 50A), a finding that is robust under different values for each parameter (FIG. 50B). These data suggest that rarer cell types may be critical players in immune cell-cell communication networks despite their low numbers.

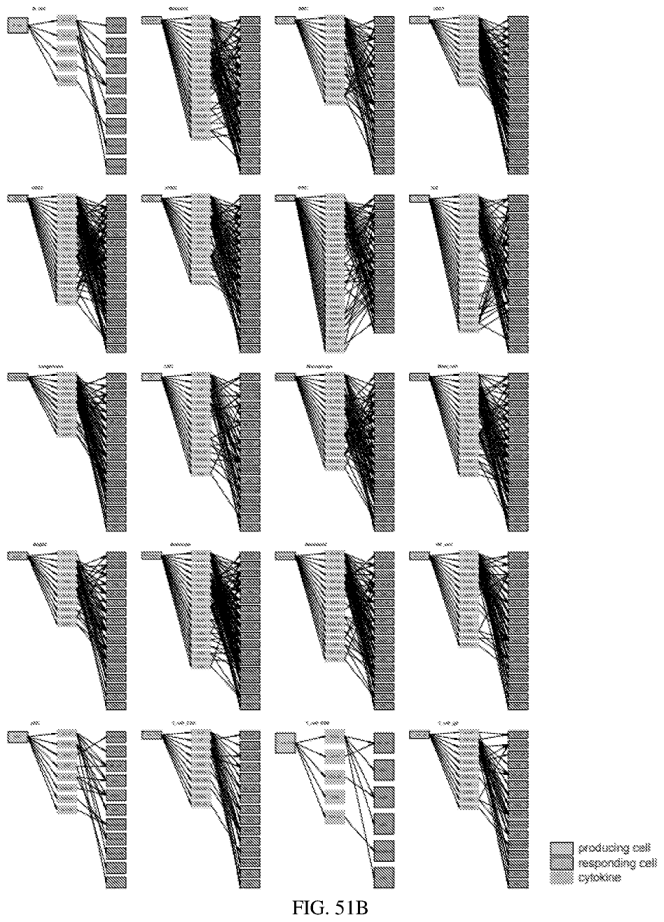

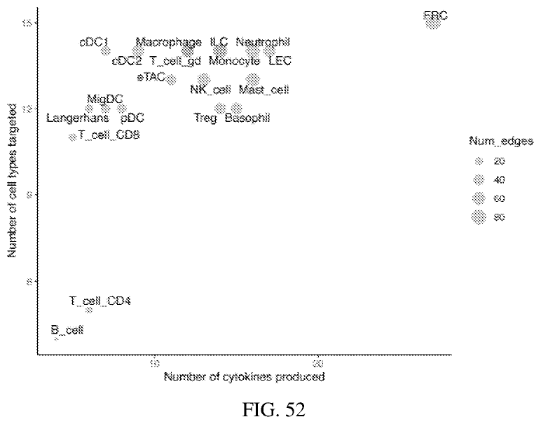

Based on cytokine production, inferred from the abundance of transcripts encoding each cytokine, and cytokine response, obtained from a global analysis in FIG. 27A, a cell-cell interactome charting possible cell-cell communication network routes was built (FIG. 30C, FIG. 30D, FIG. 51). These data support that FRCs can influence many cell types via many produced cytokines (FIG. 30C). cDC1 (and other cell types) can also affect a large number of cell types, but in contrast to FRCs, via a limited number of cytokines, most prominently IL-1α/β that triggers responses in many cell types (FIG. 30D, FIG. 52). Thus, an interactome that reveals the diverse ways by which cells can influence other cell types via the cytokine network is provided.

### Immune Response Enrichment Analysis

Transcriptomic analyses of immune processes and diseases have become standard approaches in most studies, thus generating massive public datasets^{34,35}. However, transcriptomic data do not reveal the factors triggering the observed cell states and their functions, calling for an approach for inferring cytokine responses and revealing cell-cell communication networks based on cytokine-induced gene expression programs. Previous studies have inferred cell-cell communications in transcriptomic studies using ligand and receptor expression^{36,37}, but cytokine receptor gene expression alone is not an accurate predictor of cytokine responses as the ligands or downstream pathways may not be present for a particular cell. A more precise approach considers whether a cell expresses the signature of a cytokine as defined in the immune dictionary described herein.

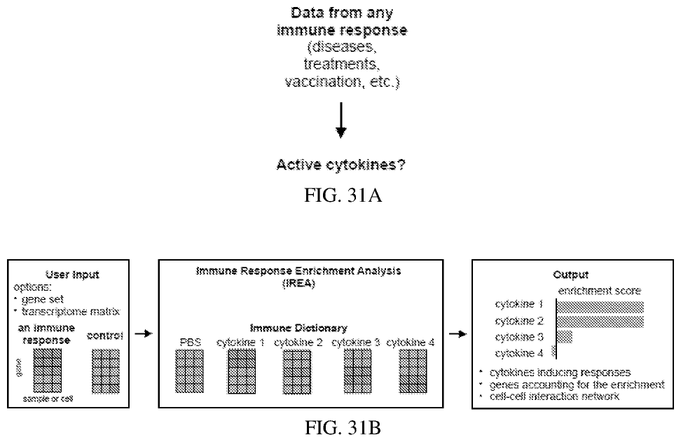

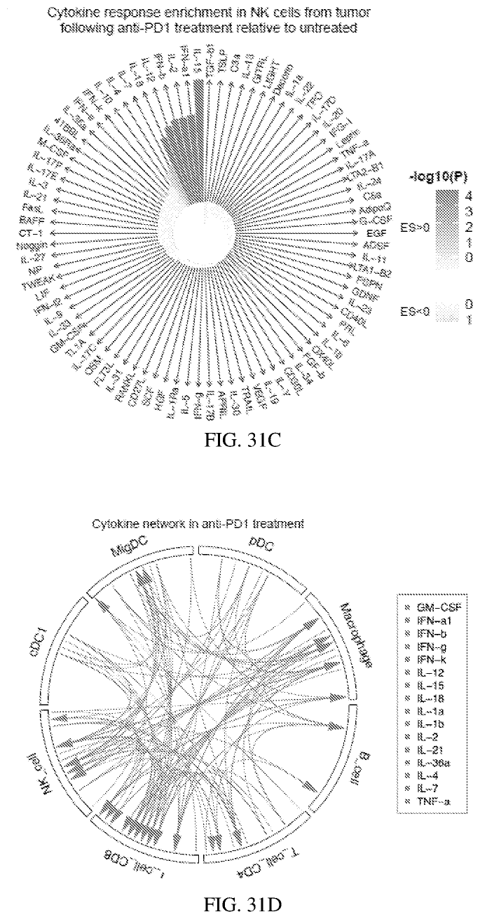

To infer cellular responses to cytokines from any transcriptomic data, Immune Response Enrichment Analysis (IREA)—the companion software for the Immune Dictionary—was created. IREA implements statistical tests to assess the levels of cytokine responses in a given cell type by calculating the similarity of its gene expression with gene expression responses to single cytokines from the dictionary. These results can then be used to derive cell-cell communication networks that explain the observed immune response (FIG. 31A, FIG. 31B). IREA was applied to a published single-cell transcriptomic dataset from tumors of mice treated with the anti-PD-1 checkpoint blockade therapy³⁸. It was found that IL-12 is produced by dendritic cells and macrophages, and it was inferred that the cell types that respond most strongly to IL-12 are T cells and NK cells (FIG. 31C and FIG. 31D), in agreement with prior findings that IL-12 is critical in effective checkpoint blockade immunotherapy³⁹. IREA found that the immunosuppressive cytokine transforming growth factor β (TGF-β) showed the most negative response in anti-PD-1 treated cells compared to untreated (FIG. 31C), consistent with its known role in attenuating the immune boost caused by PD-1/PD-L1 blockade⁴⁰. Several other cytokines, including IL-15, IL-2, IFN-α1 and IL-18, were also found to be produced by and act on specific cell types in the tumor (FIG. 31D). This framework thus enabled inference of the key secreted factors that triggered observed cellular responses (e.g., FIG. 31C) and generation of a molecular model of cell-cell interactions (e.g., FIG. 31D) underlying a complex immune response.

In summary, a large compendium of transcriptomic profiles in response to cytokines was created, providing the ability to generate new hypotheses for the functions of each cytokine and leading to the conclusion that the plasticity of immune cells and complexity of cytokine responses are much greater than what was previously appreciated. It was shown that each cell type has a unique response to a particular cytokine, likely reflecting cell-type specific differences in downstream signaling or transcription. Single cytokine-induced cellular polarization states were systematically delineated in each major immune cell type, and multiple polarization states that were previously unknown were uncovered. A cytokine production map and cytokine receptor expression map by cell type were also provided, providing a basis for inferring cytokine sources and highlighting the role of rare cells in the cytokine communication network. Finally, a method to infer cytokine responses and cell-cell communication networks in any immune response for which single cell transcriptomics data has been collected was developed. Since the dictionary is collected at the single-cell resolution, one can easily re-analyze the immune responses in any subpopulation of cells of interest. This study provides the first systematic cell-type-specific dictionary of cytokine responses and a basis for inferring cell-cell communication networks, which can be applied to the study and modulation of any immune response.

Differentially expressed genes in each cell type treated with each cytokine are shown in Table 1 provided herein.

### Methods

Cytokine injection: Every cytokine was obtained from at least two separate orders. To preserve cytokine activities, carrier-free cytokines were freshly reconstituted according to the manufacturer's recommendations, stored under 4° C. in sterile conditions, and used within 28 hours post-reconstitution. For each cytokine, 5 μg in 100 μl sterile PBS were injected into each animal. Wild type female C57BL/6 mice were purchased from the Jackson Laboratory, and used in studies at 11-15 week-old, analogous to young human adults. Cytokines were injected under the skin (50% subcutaneous, 50% intradermal) bilaterally in the abdominal flank of each mouse. Bilateral skin-draining inguinal lymph nodes were harvested 4 hours post-injection at 6 am-8 am and pooled for downstream processing. For each of the 86 cytokines, 3 replicates of C57BL/6 mice were performed to ensure reproducibility. The PBS control was included with every batch.

Data generation and quality assurance: Batch effects that arise from samples processed on different days are known to be a major source of artifact in transcriptomic studies. Therefore, batch-to-batch consistency was strictly ensured experimentally and then verified computationally. Specifically, the mice were ordered from the same batch and housed in the same environment. The same researchers performed the same parts of the sample processing and sequencing pipeline while following the same, highly optimized, processing procedures. All samples were processed fresh and on ice whenever possible. All experiments were harvested at 6-8 am to exclude the impact of circadian clocks on transcriptomic profiles. The number of batches was minimized whenever possible. The 3 replicates of each cytokine were processed in different batches to ensure that batch effects, if any, do not influence biological interpretations. All samples were sequenced on two sequencing runs, with the first sequencing run containing the first set of replicates and the second containing the second and third set of replicates. PBS controls were included in every batch to ensure comparability, and transcriptomic profiles of PBS samples from different batches were computationally compared to verify batch-to-batch consistency. Briefly, Euclidean distance between transcriptomes of each pair of cells were compared to ensure that the within-batch distance and between-batch distance are comparable (FIG. 33A).

Lymph node processing for single-cell experiments: An optimized pipeline for viable cell recovery and sample multiplexing was used to process lymph nodes for scRNA-seq. Lymph nodes were enzymatically digested using a protocol that maximizes the recovery of myeloid and stromal cells while maintaining high viability, as previously described⁴¹. Briefly, lymph nodes were placed in RPMI with Collagenase IV, Dispase, and DnaseI in 37° C. and cells were harvested once they detached. The cells were then resuspended in PBS with BSA and EDTA, and placed on ice. A biotinylated anti-CD3 and anti-CD19 antibody cocktail was incubated with the cells, then streptavidin beads were added, and the samples were processed using MACS MS columns according to the manufacturer's protocol. After cell sorting, a small fraction of the CD3+ or CD19+ cells was pooled with CD3−/CD19− cells for optimal representation of all cell types, and proceeded immediately to scRNA-seq.

Single-cell RNA-sequencing: Cell hashing was used to combine multiple samples into the same single-cell emulsion channel⁴². The mouse cells obtained from different stimulation conditions were stained with TotalSeq™ antibodies (BioLegend anti-mouse Hashtag #1-7), washed 5 times and pooled in PBS with 0.04% BSA according to the manufacturer's protocol. Next, 55,000 cells were loaded onto a 10× Genomics Chromium™ instrument (10x Genomics) according to the manufacturer's instructions. The scRNA-seq libraries were processed using Chromium Single Cell 3' Library & Gel Bead v3 Kit (10× Genomics) with modifications for generating hashtag libraries⁴². Quality control for amplified cDNA libraries and final sequencing libraries was performed using Bioanalyzer High Sensitivity DNA Kit (Agilent). scRNA-seq and hashing libraries were normalized to 4 nM concentration and pooled. The pooled libraries were sequenced on the NovaSeq S4 or SP platform, targeting an average sequencing depth of 20,000 reads per cell for gene expression libraries and 5,000 reads per cell for hashtag libraries.

scRNA-seq data pre-processing: The raw bcl sequencing data was processed using the Cell Ranger (version 3.0) Gene Expression pipeline (10× Genomics), including demultiplexing and alignment. Sequencing reads were aligned to the mm10 mouse reference genome (version 3.0.0) and transcriptomic count matrices were assembled. Hashtag library FASTQ files were processed through the CITE-seq-Count tool (version 1.4.3, https://github.com/Hoohm/CITE-seq-Count). Gene expression and hashtag were matched using the MULTIseqDemux function of the Seurat R package (version 3.2.2)⁴³. Cells with multiple hashtags were considered multiplets (e.g., doublets, triplets) and were excluded from further analysis. The Seurat R pipeline was used to perform quality control to include only cells with >500 genes, >1,000 UMIs, and <10% mitochondrial gene content. The expression matrix was normalized by normalizing the gene expression measurements by the total expression per cell, multiplied by a scale factor of 10,000, and log-transformed.

For the initial global analysis of all cells, the top 3000 variable genes were selected for dimensionality reduction analysis. Principal component analysis (PCA) was then used to denoise and to find a lower-dimensional representation of the data. The top 75 principal components (PCs) were used for clustering and for visualization using a tSNE map⁴⁴. Clusters were identified using the Louvain clustering algorithm. This results in a total of 66 global (level-1) clusters. Potential multiplets were removed by removing the cells with the top 2% gene counts in each cluster. Because different cell types have different numbers of genes detected on average, this step was done at the cluster level as opposed to for all data. For each level-1 cluster of cells, another round of clustering (level-2) was then performed to further verify the identity of each cluster and remove potential doublets. This results in a total of 180 level-2 clusters. Cell type identity of each level-2 cluster was assigned based on known marker genes. Clusters enriched for marker genes of multiple cell types were considered multiplets and removed.

Quantitative measures of reproducibility across biological replicates: A gene expression vector for each biological replicate is created for each cytokine stimulation condition in a given cell type by taking a difference between the average expression vector of cytokine-treated cells and average expression vector of PBS-treated cells. The same numbers of cells were sampled for each biological replicate to ensure uniformity. Genes that were found to be significantly differentially expressed compared to PBS controls were included. Pairwise Pearson correlation coefficients were then calculated on these vectors.

Quantification of magnitude of cell-type-specific transcriptomic responses to each cytokine: For each cytokine, the magnitude of differential gene expression between cytokine-stimulated conditions and PBS controls of the same cell type was computed. A maximum of 100 cells from each condition were sampled. A reference vector was created by computing the centroid vector of PBS-treated cells in each cell type. A background distribution was generated by computing the divergence between PBS-treated cells relative to the reference vector. For each cytokine, a distribution of cytokine treated cells relative to the reference vector were computed. The magnitude of differential expression is calculated as the Euclidean distance between cytokine response vectors and background vectors in each cell type on the top 150 principal components. Statistical analysis was performed using the two-sided Wilcoxon Rank-Sum Test on these two distributions and effect size was computed by the mean difference between distributions, normalized to be between 0 and 1. This differential expression is shown as a heatmap for all cell types (FIG. 27C).

Identification of gene expression programs: Gene programs (GPs) were constructed by the non-negative matrix factorization (NMF) algorithm¹⁹ using the R package NMFN. Genes associated with tissue dissociation⁴⁵ and cell cycle were removed, as well as mitochondrial genes, Rps/Rpl genes, and those expressed in fewer than 10 cells prior to running NMF. For the identification of cell-type-specific GPs in response to certain cytokines, only cells treated with the cytokines of interest were included in the count matrix. Some GPs correspond to cell type identity while others correspond to the cellular response to cytokine stimulation. A total of 40 GPs was chosen for the individual cytokine analysis to account for both GPs related to cell type identity and cell type states, and a total of 10 GPs were chosen for the individual cell-type analysis. To identify GPs corresponding to cell state changes due to cytokine stimulation, a Wilcoxon rank sum test was used to compute GPs that were statistically significantly different between stimulation condition and PBS condition. Genes with the highest weight for each GP were used to identify cellular activities of the corresponding GP.

Identification of cellular polarization states: For each cell type, sub-clustering was performed on all cells on discriminating genes, defined as genes with a large log₂ fold change (between 0.4-1.5 depending on cell type) in any cytokine treated cells compared to PBS treated cells. Genes associated with tissue dissociation⁴⁵, cell cycle, mitochondrial genes, and Rps/Rpl genes were removed. Principal component analysis was then performed, and the cells were visualized on UMAP²⁹. The proportion of cells falling into each cluster was calculated for each cytokine or PBS control. Polarization states were identified based on the two criteria: 1) cell clusters with significantly (FDR adjusted p<0.01) more than the expected number of cytokine-stimulated cells using a hypergeometric test, 2) manual verification of biological relevance of the highly expressed genes in the cluster and cytokines inducing the changes. The most strongly polarized states are summarized in FIG. 29. The complete landscape, including less-strongly polarized states, in each cell type can be found in FIG. 36-FIG. 49.

Cytokine production map: A map of cell-type-specific production of cytokines in the 86 cytokine simulations or PBS controls was obtained from this study. To account for the imbalance in the number of cells obtained from each cell type, the calculation was done on subsampled data that have the same number of cells in each cell type. The subsamples were obtained from all conditions (PBS or cytokine treated) to provide a map of cytokine expression under all cytokine stimulation conditions. The gene expression was then normalized to the cell type that has the maximum expression level. A cytokine was considered expressed in a cell type if more than 0.05 expression units were detected. This analysis was performed on different detection thresholds to ensure the robustness of the results (FIG. 50A and FIG. 50B).

Cell-cell interactome network construction: A cell-cell interactome network was constructed to chart most of the possible cytokine-mediated cell-cell communication routes. The network was constructed such that the source and sink nodes are cell populations, and intermediate nodes are cytokines. The edges between source nodes and cytokine nodes are determined based on the detectability of cytokine mRNA in the cell population, and the edges between cytokine nodes and sink nodes are determined based on the responsiveness of cell type to each cytokine as shown in FIG. 27C. The network was plotted separately for each source node for ease of interpretability.

Immune response enrichment analysis (IREA) software: Two types of IREA analysis options are offered to assess cytokine response in user's data depending on the input, which can be 1) a list of genes or 2) gene expression matrix. Cell type is specified by the user. User data is then compared to the transcriptional cytokine responses of the same cell type from the Immune Dictionary using the following methods: 1) For gene set input, gene set scores are first found by summing the normalized expression value of all genes in the gene set in each of the cytokine-treated cells or PBS-treated cells. Statistical significance is assessed by a two-sided Wilcoxon rank-sum test between gene set scores on cytokine-treated cells and gene set scores on PBS-treated cells, and an FDR correction is applied to all cytokine calculations. Enrichment can also be calculated using the hypergeometric test on significantly differentially expressed genes (FDR<0.01 between cytokine-treated cells and PBS-treated cells), which is a method commonly used in pathway analysis. 2) For gene expression matrix input, the expression matrices are first normalized such that the total expression per cell sums to 10,000 units; the expression is then log-transformed. Genes giving significant contribution to the enrichment score, with the default being those having an average of more than 0.25 expression values, were included. Next, the projection score is calculated by finding the cosine similarity score between user input and cytokine-treated or PBS-treated cells. Statistical significance is assessed by a two-sided Wilcoxon rank-sum test between projection scores on cytokine-treated cells and projection scores on PBS-treated cells, and an FDR correction is applied to all cytokine calculations. Effect size is the mean difference between projection scores on cytokine-treated cells and on PBS-treated cells. The effect size and p-value can then be visualized using a compass-plot as shown in FIG. 31C.

Statistical analysis: The statistical tests used are described for each analysis in their corresponding text. Two-sided statistical tests were used unless otherwise specified. FDR adjustment is made for the analyses where multiple hypothesis testing applies.

### REFERENCES

1. Arai, K. I. et al. Cytokines: coordinators of immune and inflammatory responses. Annu. Rev. Biochem. 59, 783-836 (1990).
2. Dranoff, G. Cytokines in cancer pathogenesis and cancer therapy. Nat. Rev. Cancer 4, 11-22 (2004).
3. Ye, Q., Wang, B. & Mao, J. The pathogenesis and treatment of the ‘Cytokine Storm’ in COVID-19. Journal of Infection vol. 80 607-613 (2020).
4. Pestka, S., Krause, C. D. & Walter, M. R. Interferons, interferon-like cytokines, and their receptors. Immunol. Rev. 202, 8-32 (2004).
5. Vignali, D. A. A. & Kuchroo, V. K. IL-12 family cytokines: immunological playmakers. Nat. Immunol. 13, 722-728 (2012).
6. Sims, J. E. & Smith, D. E. The IL-1 family: regulators of immunity. Nat. Rev. Immunol. 10, 89-102 (2010).
7. Locksley, R. M., Killeen, N. & Lenardo, M. J. The TNF and TNF receptor superfamilies: integrating mammalian biology. Cell 104, 487-501 (2001).
8. Akdis, M. et al. Interleukins (from IL-1 to IL-38), interferons, transforming growth factor β, and TNF-α: Receptors, functions, and roles in diseases. J. Allergy Clin. Immunol. 138, 984-1010 (2016).
9. Korn, T., Bettelli, E., Oukka, M. & Kuchroo, V. K. IL-17 and Th17 Cells. Annu. Rev. Immunol. 27, 485-517 (2009).
10. Urrutia, A. et al. Standardized Whole-Blood Transcriptional Profiling Enables the Deconvolution of Complex Induced Immune Responses. Cell Rep. 16, 2777-2791 (2016).
11. Subramanian, A. et al. A Next Generation Connectivity Map: L1000 Platform and the First 1,000,000 Profiles. Cell 171, 1437-1452.e17 (2017).
12. Xue, J. et al. Transcriptome-based network analysis reveals a spectrum model of human macrophage activation. Immunity 40, 274-288 (2014).
13. Macosko, E. Z. et al. Highly Parallel Genome-wide Expression Profiling of Individual Cells Using Nanoliter Droplets. Cell 161, 1202-1214 (2015).
14. Trapnell, C. Defining cell types and states with single-cell genomics. Genome Res. 25, 1491-1498 (2015).
15. Merad, M., Ginhoux, F. & Collin, M. Origin, homeostasis and function of Langerhans cells and other langerin-expressing dendritic cells. Nat. Rev. Immunol. 8, 935-947 (2008).
16. Gardner, J. M. et al. Deletional tolerance mediated by extrathymic Aire-expressing cells. Science 321, 843-847 (2008).
17. Zhou, T. et al. IL-18BP is a secreted immune checkpoint and barrier to IL-18 immunotherapy. Nature 583, 609-614 (2020).
18. Yasuda, K., Nakanishi, K. & Tsutsui, H. Interleukin-18 in Health and Disease. Int. J. Mol. Sci. 20, (2019).
19. Lee, D. D. & Sebastian Seung, H. Learning the parts of objects by non-negative matrix factorization. Nature vol. 401 788-791 (1999).
20. Dinarello, C. A. Interleukin-1. Cytokine Growth Factor Rev. 8, 253-265 (1997).
21. Dinarello, C. A. Biology of interleukin 1. FASEB J. 2, 108-115 (1988).
22. Weber, A., Wasiliew, P. & Kracht, M. Interleukin-1 (IL-1) pathway. Sci. Signal. (2010).
23. Dinarello, C. A. & Wolff, S. M. The role of interleukin-1 in disease. N. Engl. J. Med. 328, 106-113 (1993).
24. Ozaki, K. & Leonard, W. J. Cytokine and cytokine receptor pleiotropy and redundancy. J. Biol. Chem. 277, 29355-29358 (2002).
25. Murray, P. J. Macrophage Polarization. Annu. Rev. Physiol. 79, 541-566 (2017).
26. Brown, C. C. et al. Transcriptional Basis of Mouse and Human Dendritic Cell Heterogeneity. Cell 179, 846-863.e24 (2019).
27. Crinier, A. et al. High-Dimensional Single-Cell Analysis Identifies Organ-Specific Signatures and Conserved NK Cell Subsets in Humans and Mice. Immunity 49, 971-986.e5 (2018).
28. Lavin, Y. et al. Tissue-resident macrophage enhancer landscapes are shaped by the local microenvironment. Cell 159, 1312-1326 (2014).
29. Becht, E. et al. Dimensionality reduction for visualizing single-cell data using UMAP. Nat. Biotechnol. (2018) doi:10.1038/nbt.4314.
30. Dinarello, C. A. Overview of the IL-1 family in innate inflammation and acquired immunity. Immunol. Rev. 281, 8-27 (2018).
31. Freud, A. G., Mundy-Bosse, B. L., Yu, J. & Caligiuri, M. A. The Broad Spectrum of Human Natural Killer Cell Diversity. Immunity 47, 820-833 (2017).
32. Park, K. et al. The transcription factor NR4A3 controls CD103+ dendritic cell migration. J. Clin. Invest. 126, 4603-4615 (2016).
33. Krishnamurty, A. T. & Turley, S. J. Lymph node stromal cells: cartographers of the immune system. Nat. Immunol. 21, 369-380 (2020).
34. Stubbington, M. J. T., Rozenblatt-Rosen, O., Regev, A. & Teichmann, S. A. Single-cell transcriptomics to explore the immune system in health and disease. Science 358, 58-63 (2017).
35. Papalexi, E. & Satija, R. Single-cell RNA sequencing to explore immune cell heterogeneity. Nat. Rev. Immunol. 18, 35-45 (2018).
36. Vento-Tormo, R. et al. Single-cell reconstruction of the early maternal—fetal interface in humans. Nature 563, 347-353 (2018).
37. Ramilowski, J. A. et al. A draft network of ligand—receptor-mediated multicellular signalling in human. Nat. Commun. 6, 7866 (2015).
38. Gubin, M. M. et al. High-Dimensional Analysis Delineates Myeloid and Lymphoid Compartment Remodeling during Successful Immune-Checkpoint Cancer Therapy. Cell 175, 1443 (2018).
39. Garris, C. S. et al. Successful Anti-PD-1 Cancer Immunotherapy Requires T Cell-Dendritic Cell Crosstalk Involving the Cytokines IFN-γ and IL-12. Immunity 49, 1148-1161.e7 (2018).
40. Mariathasan, S. et al. TGFβ attenuates tumour response to PD-L1 blockade by contributing to exclusion of T cells. Nature 554, 544-548 (2018).
41. Fletcher, A. L. et al. Reproducible Isolation of Lymph Node Stromal Cells Reveals Site-Dependent Differences in Fibroblastic Reticular Cells. Frontiers in Immunology vol. 2 (2011).
42. Stoeckius, M. et al. Cell Hashing with barcoded antibodies enables multiplexing and doublet detection for single cell genomics. Genome Biol. 19, 224 (2018).
43. Stuart, T. et al. Comprehensive Integration of Single-Cell Data. Cell 177, 1888-1902.e21 (2019).
44. van der Maaten, L. & Hinton, G. Visualizing Data using t-SNE. J. Mach. Learn. Res. 9, 2579-2605 (2008).
45. van den Brink, S. C. et al. Single-cell sequencing reveals dissociation-induced gene expression in tissue subpopulations. Nat. Methods 14, 935-936 (2017).
46. Subramanian, A. et al. Gene set enrichment analysis: a knowledge-based approach for interpreting genome-wide expression profiles. Proc. Natl. Acad. Sci. U.S.A. 102, 15545-15550 (2005).

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

Lengthy table referenced here
US20230407397A1-20231221-T00001
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20230407397A1). An electronic copy of the table will also be available from the USPTO upon request and payment of the fee set forth in 37 CFR 1.19(b)(3).

What is claimed is:

1. A method for determining signatures of immune responses to immunomodulating agents, the method comprising steps of:
(a) administering an immunomodulating agent into a population of cells, wherein the population of immune cells comprises multiple cell types;
(b) harvesting the population of cells;
(c) profiling gene expression in each cell within the population of cells;
(d) correlating cell type and gene expression to compute cell-type-specific gene expression signatures;
(e) repeating steps (a)-(d) for additional immunomodulating agents; and
(f) comparing the cell-type-specific gene expression signatures across immunomodulating agents.

2. The method of claim 1, wherein the immunomodulating agent is a cytokine.

3. The method of claim 2, wherein the cytokine is an IL-1 family cytokine, an IL-6 family cytokine, an IL-10 family cytokine, an IL-12 family cytokine, an IL-17 family cytokine, a common γ chain family cytokine, a common β chain family cytokine, an interferon, or a TNF family cytokine.

4. The method of claim 1, wherein the immunomodulating agent is a growth factor.

5. The method of claim 1, wherein the immunomodulating agent is a hormone.

6. The method of claim 1, wherein the immunomodulating agent is a vaccine adjuvant.

7. The method of claim 1, wherein the population of cells is a population of human cells.

8. The method of claim 1, wherein the cell types are selected from the group consisting of monocytes, NK cells, T cells, macrophages, lymphatic cells, innate lymphoid cells, neutrophils, blood endothelial cells, fibroblastic reticular cells, mast cells, basophils, Langerhans cells, and dendritic cells.

9. The method of claim 8, wherein the T cells are CD4 T cells, CD8 T cells, regulatory T cells, or gamma delta T cells.

10. The method of claim 7, wherein the dendritic cells are conventional type 1 dendritic cells, conventional type 2 dendritic cells, migratory dendritic cells, or plasmacytoid dendritic cells.

11. The method of claim 1, wherein the population of cells is in vivo.

12. The method of claim 1, wherein the population of cells is in a mammal.

13. The method of claim 12, wherein the mammal is a mouse.

14. The method of claim 12, wherein the mammal is a human.

15. The method of claim 1, wherein the step of harvesting is performed at least 2 hours, at least 3 hours, at least 4 hours, at least 5 hours, or at least 6 hours after the step of administering.

16. The method of claim 1, wherein the step of harvesting is performed 4 hours after the step of administering.

17. The method of any one of claims 11-14, wherein the step of harvesting the population of immune cells comprises harvesting and processing cells from draining lymph nodes.

18. The method of claim 1, wherein the step of profiling gene expression comprises performing single-cell RNA sequencing.

19. The method of claim 1, wherein the step of profiling gene expression comprises bulk gene expression profiling.

20. The method of claim 1, wherein the step of profiling gene expression comprises single-cell gene expression profiling.

21. The method of claim 1, wherein the step of profiling gene expression comprises protein expression profiling.

22. The method of claim 1, wherein the step of profiling gene expression comprises chromatin accessibility profiling.

23. The method of claim 1, wherein the method further comprises identifying polarization states of cell types within the population of immune cells in response to immunomodulating agents.

24. The method of claim 1, further comprising:
determining a target gene expression profile; and
determining, using at least some of the signatures of immune response, at least one immunomodulating agent associated with the target gene expression profile.

25. The method of claim 24, wherein determining the at least one immunomodulating agent associated with the target gene expression profile is performed by regressing the at least some of the signatures of immune response against the target gene expression profile using a regression technique.

26. The method of claim 25, wherein the regression technique is a regularized linear regression technique.

27. The method of claim 26, wherein the regularized linear regression technique is a ridge regression technique, a least absolute shrinkage and selection operator (LASSO) regression technique, or an elastic net regression technique.

28. The method of claim 24, wherein determining the at least one immunomodulating agent associated with the target gene expression profile is performed by correlating at least one signature of immune response with the target expression profile.

29. A method for treating a disease or disorder in a subject in need thereof, the method comprising administering one or more immunomodulating agents that drive an immune response within the subject in combination with a treatment for the disease or disorder, wherein the immune response is associated with increased efficacy of the treatment.

30. The method of claim 29, wherein the disease or disorder is a proliferative disease, an infectious disease, an inflammatory disease, an autoimmune disease, or allergies.

31. The method of claim 30, wherein the disease or disorder is cancer

32. The method of claim 31, wherein the immune response is induced in NK cells.

33. The method of claim 32, wherein the immune response comprises enhancing NK cell proliferation.

34. The method of claim 33, wherein the immunomodulating agent is a cytokine.

35. The method of claim 33, wherein the immunomodulating agent is selected from the group consisting of IL18, IL15, IFNb, IL2, IL12, IL7, IL33, IL27, IFNk, Cardiotrophin-1, IL36α, GM-CSF, IFNe, IL3, IL11, OSM, 41BBL, IL9, M-CSF, CD40L, Neuropoietin, TL1A, IL21, LIF, IL4, FasL, IL17F, IL17A, IL10, IL17E, IL5, IFNI2, LIGHT, IFNg, Noggin, IL24, IL17C RANKL IL13, BAFF, Persephin, IL23, Flt3I, G-CSF, APRIL, IL36RA, VEGF, GDNF, IL31, LTA1-B2, GITRL, IL6, CD30L, HGF, OX40L, CD27L, FGF-basic, Resistin, IL17B, Leptin, IL30, TRAIL, SCF, IL17D, IL-Y, IGF-I, EGF, IL20, and LTA2-B1.

36. The method of claim 33, wherein the immunomodulating agent is IL18.

37. The method of claim 31, wherein the immune response is induced in dendritic cells.

38. The method of claim 37, wherein the immune response comprises induction of Ccr7, MHC-II, Cd80, Cd83, Cd86, or Mfge8 expression.

39. The method of any one of claim 31, wherein the immune response is induced in macrophages.

40. The method of claim 39, wherein the immune responses comprises induction of iNos or MHC-II expression in M1-like macrophages.

41. The method of claim 39, wherein the immune response comprises elevated expression of Arg1 or IL10 in M2-like macrophages.

42. The method of claim 39, wherein the immune response comprises decreased expression of M1-like macrophages.

43. The method of claim 39, wherein the immune response comprises decreased expression of M1-like macrophages.

44. The method of claim 30, wherein the immune response comprises decreased antigen presentation molecules on B cells.

45. The method of claim 30, wherein the disease or disorder is rheumatoid arthritis.

46. The method of claim 45, wherein the immune response comprises reduction of TNF expression.

47. The method of claim 30, wherein the disease or disorder is psoriasis.

48. The method of claim 47, wherein the immune response comprises reduction of IL23 expression.

49. The method of claim 29, wherein the disease or disorder is an allergic reaction.

50. The method of claim 49, wherein the immune response comprises decreased expression of IL4, IL5, IL13, or IL33.

51. The method of claim 50, wherein the immune response comprises decreased activation of mast cells or basophils.

52. The method of claim 29, wherein the immune response comprises induction of Granzyme B (GZMB).

53. The method of claim 52, wherein Granzyme B is induced in T cells and NK cells.

54. The method of claim 29, wherein the immune response comprises suppression of transforming growth factor beta (TGF-β).

55. The method of any one of claims 52-54, wherein the treatment is an anti-tumor therapy.

56. The method of any one of claims 52-54, wherein the treatment is an anti-viral therapy.

57. The method of any one of claims 52-56, wherein the treatment is checkpoint blockade therapy.

58. The method of claim 29, wherein the immunomodulating agent is a vaccine.

59. The method of claim 58, wherein the immune response comprises regulation of IFNa, IFNb, TFNa, IL15, IL18, IL1a, IFNk, IL36α, IFNg, Flt3I, OX40L, BAFF, IFNg, Flt3I, IL6, IL27, IL36-RA, IL34 or GM-CSF.

60. A method of polarizing a population of cells, the method comprises stimulating the cells with IL7r, Gzmb, Isg15, Klra4, Srm, or Mcm5.

61. The method of claim 60, wherein the cell are NK cells.

62. A method of expanding a population of cells, the method comprising stimulating the cells with an immunomodulating agent.

63. The method of claim 62, wherein the cells are NK cells.

64. The method of claim 62, wherein the immunomodulating agent is a cytokine.

65. The method of claim 62, wherein the immunomodulating agent is selected from the group consisting of IL18, IL15, IFNb, IL2, IL12, IL7, IL33, IL27, IFNk, Cardiotrophin-1, IL36α, GM-CSF, IFNe, IL3, IL11, OSM, 41BBL, IL9, M-CSF, CD40L, Neuropoietin, TL1A, IL21, LIF, IL4, FasL, IL17F, IL17A, IL10, IL17E, IL5, IFNI2, LIGHT, IFNg, Noggin, IL24, IL17C RANKL IL13, BAFF, Persephin, IL23, Flt3I, G-CSF, APRIL, IL36RA, VEGF, GDNF, IL31, LTA1-B2, GITRL, IL6, CD30L, HGF, OX40L, CD27L, FGF-basic, Resistin, IL17B, Leptin, IL30, TRAIL, SCF, IL17D, IL-Y, IGF-I, EGF, IL20, and LTA2-B1.

66. A method of treating cancer, the method comprising administering IL18 to a subject in need thereof in combination with a cancer therapy.

67. A method for treating cancer, the method comprising administering an anti-cancer therapy in combination with inducing GZMB expression in a subject in need thereof.

68. The method of claim 67, wherein GZMB is induced in T cells and NK cells.

69. A method for treating a viral infection, the method comprising administering an anti-viral treatment in combination with inducing GZMB expression in a subject in need thereof.

70. The method of claim 69, wherein GZMB is induced in T cells and NK cells.

71. A method of vaccination, the method comprising administering a vaccine and stimulating IFNa, IFNb, TFNa, IL15, IL18, IL1a, IFNk, IL36α, IFNg, Flt3I, OX40L, BAFF, IFNg, Flt3I, IL6, IL27, IL36-RA, IL34, or GM-CSF in a subject in need thereof

72. The method of any one of claims 1-28, further comprising using the cell-type-specific gene expression signatures obtained in steps (a)-(f) to identify cells responding to these agents in single cell expression profiles from tissues of individuals with a disease or undergoing therapy for a disease.

73. The method of claim 1, wherein the cell types are selected from the group consisting of B cells, monocytes, NK cells, T cells, macrophages, lymphatic cells, innate lymphoid cells, neutrophils, blood endothelial cells, fibroblastic reticular cells, mast cells, basophils, Langerhans cells, and dendritic cells.

74. The method of claim 1, wherein step (d) further comprises calculating expression signatures associated with each immune-modulating agent relative to a control population of immune cells that has not been administered an immunomodulating agent.

75. The method of claim 24, wherein determining the at least one immunomodulating agent associated with the target gene expression profile is performed using a Wilcoxon rank-sum test.
